(19) United States
(12) Patent Application Publication
(10) Pub. No.: US 2024/0050602 A1
(43) Pub. Date: Feb. 15, 2024

(54) PARTICLES FUNCTIONALIZED WITH THERAPEUTIC RADIOISOTOPES AND METHODS OF MAKING AND USE THEREOF

(71) Applicant: University of Virginia Patent Foundation, Charlottesville, VA (US)

(72) Inventors: James Radford Stone, Charlottesville, VA (US); Kiel Douglas Neumann, Charlottesville, VA (US)

(21) Appl. No.: 18/264,347

(22) PCT Filed: Feb. 7, 2022
(86) PCT No.: PCT/US2022/015519

§ 371 Date: Aug. 4, 2023

Related U.S. Application Data
(60) Provisional application No. 63/147,161, filed on Feb. 8, 2021.

(51) Int. Cl. A61K 51/12; A61K 51/02; A61P 35/00; A61N 5/10; G21G 4/08

(52) U.S. Cl. CPC A61K 51/1251; A61K 51/025; A61P 35/00; A61N 5/1007; G21G 4/08; A61N 2005/1021

(57) ABSTRACT

Some embodiments relate to therapeutic radioisotopic particles. In some embodiments, the therapeutic particles are radiolabeled with therapeutic radioisotopes. In some embodiments, the therapeutic particles can be in the treatment of cancer of the liver. In some embodiments, the therapeutic particles are radiolabeled with therapeutic radioisotopes. In some embodiments, the therapeutic radioisotope is directly coupled to a surface of a substrate of the particle.

### CROSS REFERENCE TO RELATED APPLICATIONS

The present application claims the benefit of priority to U.S. Provisional Patent Application No. 63/147,161, filed Feb. 8, 2021. The disclosure of the foregoing application is hereby incorporated by reference in its entirety. Any and all applications for which a foreign or domestic priority claim is identified in the Application Data Sheet as filed with the present application are hereby incorporated by reference under 37 CFR 1.57.

### FIELD

The present disclosure pertains to therapeutic particles comprising therapeutic radioisotopes, methods of making such particles, and methods of using such particles for cancer treatment.

### BACKGROUND

### Description of the Related Art

One approach to the treatment of patients with certain cancers is to introduce radioactive isotopes into the patient's circulatory system. A measured amount of radioactive isotopes are injected into the patient such that they accumulate at the site of the cancer sufficiently to treat the cancer.

### SUMMARY

In one method for the radio-treatment of cancer, radioactive microspheres are delivered to a point in the vasculature of a patient such that they will be carried by blood flow into a tissue of interest. Once at the tissue of interest, they lodge in the capillaries and deliver a dose of therapeutic radiation. This treatment has been termed selective internal radiation therapy (SIRT). The goal is to have a sufficient dose of radiation dose to cause localised tissue death of cancerous tissues.

Some embodiments disclosed herein pertain to therapeutic radioisotopic particles (e.g., nanoparticles, microparticles, or combinations thereof) that are functionalized with therapeutic isotopes about their periphery. In some embodiments, the therapeutic particles are decorated with radioactive isotopes allowing their tuning and, in some alternatives, functionalization with targeting agents. In some embodiments, the use of therapeutic radioisotopic particles as disclosed herein can allow more accurate radiation dosing, more effective treatment, and/or lower incidences of side effects for patients.

As disclosed elsewhere herein, some embodiments pertain to a particle (e.g., nanoparticle, microparticle, or combinations thereof) comprising at least one therapeutic radioisotope and a substrate comprising an inorganic material. In some embodiments, the substrate comprises non-metal atoms bonded to metalloid atoms and/or metal atoms. In some embodiments, the substrate comprises a core extending to a surface, the core comprising a first portion of the metalloid or metal atoms bonded to the non-metal atoms and the surface comprising a second portion of the metalloid or metal atoms bonded to the non-metal atoms. In some embodiments, the therapeutic radioisotope is chemically bonded through at least a portion of the non-metal atoms at the surface of the substrate to the substrate. In some embodiments, the therapeutic radioisotope is bound directly to the substrate through at least a portion of the non-metal atoms at the surface of the substrate. In some embodiments, the therapeutic radioisotope is chemically bonded to the substrate at least in part through a bridging metal atom that is chemically bonded to at least a portion of the non-metal atoms at the surface of the substrate. In some embodiments, the therapeutic radioisotope is chemically bonded through an inorganic bridge comprising non-metal atoms of the surface of the substrate. In other embodiments, the therapeutic radioisotope may be an atom of the substrate (e.g., it is found in the substrate). In some embodiments, the substrate comprises a substantially homogeneous mixture of constituent chemical elements. In some embodiments, the surface comprises at least a portion of the constituent chemical elements. In some embodiments, the non-metal atoms are oxygen atoms. In some embodiments, the at least a portion of the oxygen atoms at the surface of the substrate are provided as hydroxyl groups.

Some embodiments pertain to a therapeutic radioisotopic particle (e.g., nanoparticle, microparticle, or combinations thereof) comprising an inorganic substrate with a surface and at least one therapeutic radioisotope. Some embodiments pertain to a therapeutic radioisotopic particle (e.g., nanoparticle, microparticle, or combinations thereof) comprising an inorganic substrate with a surface and at least one therapeutic radioisotope. In some embodiments, the substrate comprises at least one non-metal, a metalloid, or a metal. In some embodiments, the therapeutic radioisotope is bound to the surface of the substrate by a Lewis acid-base coordination bond to an inorganic Lewis base. In some embodiments, the non-metal atoms are oxygen atoms. In some embodiments, the inorganic base is an oxygen atom.

Some embodiments pertain to a therapeutic particle (e.g., nanoparticle, microparticle, or combinations thereof), comprising an inorganic substrate having a surface and at least one therapeutic radioisotope. Some embodiments pertain to a therapeutic particle (e.g., nanoparticle, microparticle, or combinations thereof), comprising an inorganic substrate having a surface and at least one therapeutic radioisotope. In some embodiments, the substrate comprises at least one non-metal and at least a metalloid or a metal. In some embodiments, the therapeutic radioisotope is bound to the surface of the substrate by a chemical bond to an oxygen of an inorganic species.

Some embodiments pertain to a therapeutic radioisotopic particle (e.g., nanoparticle, microparticle, or combinations thereof), comprising an inorganic substrate comprising a surface having one or more electron donating functionalities and at least one therapeutic radioisotope. Some embodiments pertain to a therapeutic radioisotopic particle (e.g., nanoparticle, microparticle, or combinations thereof), comprising an inorganic substrate comprising a surface having one or more electron donating functionalities and at least one therapeutic radioisotope. In some embodiments, the therapeutic radioisotope is bound directly to the surface and/or is bound to the surface through an inorganic bridge during preparation of the microsphere via chemical coupling with the one or more electron donating functionalities.

In some embodiments, the therapeutic radioisotope is bound to the surface via a chemical bond selected from an ionic bond, a covalent bond, or a coordinate bond. In some embodiments, the therapeutic radioisotope is bound via a coordinate bond.

Some embodiments pertain to a therapeutic radioisotopic particle (e.g., nanoparticle, microparticle, or combinations thereof), comprising an inorganic substrate with a surface and at least one therapeutic radioisotope. Some embodiments pertain to a therapeutic radioisotopic particle (e.g., nanoparticle, microparticle, or combinations thereof), comprising an inorganic substrate with a surface and at least one therapeutic radioisotope. In some embodiments, the substrate comprises at least one non-metal, a metalloid, or a transition metal oxide. In some embodiments, the therapeutic radioisotope is bound to the surface of the substrate by a Lewis acid-base coordination bond.

Some embodiments pertain to a therapeutic radioisotopic particle (e.g., nanoparticle, microparticle, or combinations thereof), comprising an inorganic substrate with a surface and at least one therapeutic radioisotope. Some embodiments pertain to a therapeutic radioisotopic particle (e.g., nanoparticle, microparticle, or combinations thereof) comprising an inorganic substrate with a surface and at least one therapeutic radioisotope. In some embodiments, the substrate comprises at least one non-metal and at least a metalloid and/or a metal. In some embodiments, the therapeutic radioisotope is bound to the surface of the substrate by a Lewis acid-base coordination bond to an inorganic Lewis base. In some embodiments, the therapeutic radioisotope is coupled to a surface of a ceramic microsphere substrate as a Lewis acid-base adduct through an inorganic Lewis base.

Any of the embodiments described above, or described elsewhere herein, can include one or more of the following features.

In some embodiments, the at least one therapeutic radioisotope is a positron emitter or a gamma emitter. In some embodiments, the at least one therapeutic radioisotope is not a positron emitter. In some embodiments, the at least one therapeutic radioisotope is not a gamma emitter.

In some embodiments, the at least one therapeutic radioisotope is alpha emitter (e.g., emits alpha particles). Alpha particles (a) are positively charged and made up of two protons and two neutrons from a radioactive atom's nucleus. Alpha particles come from the decay of the radioactive elements.

In some embodiments, the at least one therapeutic radioisotope is beta emitter (e.g., emits beta particles—negatron emission). As used herein, beta particles (0) are small, fast-moving particles with a negative electrical charge that are emitted from an atom's nucleus during radioactive decay. Beta particles are more penetrating than alpha particles, but are less damaging to living tissue and DNA because the ionizations they produce are more widely spaced. They travel farther in air than alpha particles, but can be stopped by a layer of clothing or by a thin layer of a substance such as aluminum. Some beta particles are capable of penetrating the skin and causing damage such as skin burns. However, as with alpha-emitters, beta-emitters may be used to damage tissue (e.g., cancer tissue) when ingested or injected internally. There are three types of nuclear reactions that are classified as beta decay processes. The first type (here referred to as beta decay) is also called negatron emission because a negatively charged beta particle is emitted, whereas the second type (positron emission) emits a positively charged beta particle. In electron capture, an orbital electron is captured by the nucleus and absorbed in the reaction. All these modes of decay represent changes of one in the atomic number Z of the parent nucleus but no change in the mass number A. Alpha decay is different because both the atomic and mass number of the parent nucleus decrease. As used herein, “positron emitters” are those that undergo positron emission, while “beta emitters” undergo negatron emission.

Gamma rays (y) are weightless packets of energy called photons. Unlike alpha and beta particles, which have both energy and mass, gamma rays are pure energy. Gamma rays are similar to visible light, but have much higher energy. Gamma rays are often emitted along with alpha or beta particles during radioactive decay.

In some embodiments, at least one therapeutic radioisotope is a metallic radioisotope. As disclosed herein, in some embodiments, the at least one therapeutic radioisotope is a positron or gamma emitter. In several embodiments, the positron or gamma emitter is selected from ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, ¹⁷⁷Lu, Al¹⁸F, and/or combinations thereof. In some embodiments, the therapeutic radioisotope is bound directly to the substrate at the surface and/or through an inorganic bridge. In some embodiments, the at least one therapeutic radioisotope is selected from ^{99m}Tc and ⁸⁹Zr. In some embodiments, the at least one therapeutic is ¹⁷⁷Lu.

As disclosed herein, in some embodiments, the at least one therapeutic radioisotope is an alpha emitter. In several embodiments, the alpha emitter is selected from ¹⁷⁷Lu, ⁹⁰Y¹³¹I, ⁸⁹Sr, ¹⁵³Sm, or combinations of any of the foregoing. As disclosed herein, in some embodiments, the at least one therapeutic radioisotope is a beta emitter. In several embodiments, the beta emitter is selected from ¹⁴⁹Tb, ²²³Ra, ²²⁴Ra, ²¹¹At, ²²⁵Ac, ²²⁷Th, ²¹²Bi, ²¹³Bi, ²¹²Pb or combinations of any of the foregoing. In some embodiments, the at least one therapeutic radioisotope comprises one or more of ¹⁷⁷Lu, ⁹⁰Y, ¹³¹I, ⁸⁹Sr, ¹⁴⁹Tb, ¹⁵³Sm, ²²³Ra, ²²⁴Ra, ²¹¹At, ²²⁵Ac, ²²⁷Th, ²¹²Bi, ²¹³Bi, and/or ²¹²Pb.

In some embodiments, a surface of the particle (e.g., nanoparticle, microparticle, or combinations thereof) comprises a structure of Formula (V):

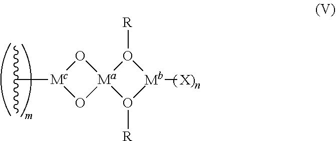

where the substrate comprises M^{c} and M^{c} is selected from Pb, Al, Si, Y, Mn, Ga, Fe, and Ti; the surface layer comprises M^{b} and M^{b} is selected from ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, Al¹⁸F, ¹⁷⁷Lu, and/or combinations thereof; each instance of R is not present or is —H; each instance of X is independently selected from is selected from —OH, ═O, and —O—; m is an integer selected from 1, 2, or 3; and n is an integer selected from 0, 1, 2, 3, or 4. In some embodiments, the substrate comprises M^{c} and M^{c} is independently selected from Pb, Al, Si, Y, Mn, Ga, Fe, Ti, Sr, Lu, I, Sm, Ra, At, Ac, Th, and Bi; m is an integer selected from 1, 2, or 3; M^{a} is either an atom of the substrate or a bridging atom and M^{a} is selected from Pb, Al, Si, Y, Mn, Ga, Fe, Ti, Sr, Lu, I, Sm, Ra, At, Ac, Th, Bi, and Sn; M^{b} is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁷⁷Lu, Al¹⁸F, and/or combinations thereof; each instance of R is either not present or is H; X is selected from —OH, ═O, and —O—; and n is an integer selected from 0, 1, 2, 3, or 4. M^{a} may either be an atom of the substrate or a bridging metal atom that chemically connects (through chemical bonds) the therapeutic radioisotope to the substrate. In some embodiments, where M^{a} is an atom of the substrate, M^{a} is selected from Pb, Al, Si, Y, Mn, Ga, Fe, and Ti. In some embodiments, M^{a} is a Sn bridging metal atom. In some embodiments, M^{c} is Al; the substrate comprises M^{a} and M^{a} is Si; M^{b} is ⁸⁹Zr; each X is independently —OH or ═O; and n is 1 or 2. In some embodiments, M^{c} and M^{a} are independently selected from Al, Si, and Y; M^{b} is ⁸⁹Zr; each X is independently —OH or ═O; and n is 1 or 2. In some embodiments, M^{b} is ⁸⁹Zr, X is —OH, and n is 2. In some embodiments, M^{c} is Si, Al, or Y; M^{a} is Sn; M^{b} is ⁹⁹mTc; each X is independently —OH or ═O; and n is 2 or 3. In some embodiments, M^{b} is ⁹⁹mTc, X is —OH, and n is 2 or 3. In some embodiments, the substrate may comprise a precursor of a therapeutic isotope that may be activated either before or after functionalization with the therapeutic radioisotope. For example, Lu, I, Sr, Sm, Ra, At, Ac, Th, Bi, and/or Pb may be activated (e.g., by neutron capture) to provide ¹⁷⁷Lu, ¹³¹, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

In some embodiments, a surface layer of the particle (e.g., nanoparticle, microparticle, or combinations thereof) comprises a structure of Formula (VIII):

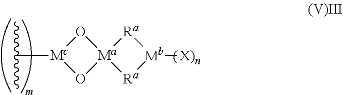

where the substrate comprises M^{a} and M^{c} and where M^{a} and M^{c} are independently selected from Pb, Al, Si, Y, Mn, Ga, Fe, and Ti; m is an integer selected from 1, 2, or 3; M^{b} is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, Sc, ⁵¹Cr, ¹⁷⁷Lu, Al¹⁸F, and/or combinations thereof; each instance of Ra is independently OH, O, or —O—Sn—O—; X is selected from —OH, ═O, and —O—; and n is an integer selected from 0, 1, 2, 3, or 4. In some embodiments, the substrate comprises M^{a} and M^{c} and where M^{a} and M^{c} are independently selected from Pb, Al, Si, Y, Mn, Ga, Fe, Ti, Sr, Lu, I, Sm, Ra, At, Ac, Th, and Bi; m is an integer selected from 1, 2, or 3; M^{b} is selected from ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴Sc, ⁵¹Cr, ¹⁷⁷Lu, Al¹⁸F, and/or combinations thereof; each instance of Ra is independently OH, O, or —O—Sn—O—; X is selected from —OH, ═O, and —O—; and n is an integer selected from 0, 1, 2, 3, or 4. In some embodiments, the Sn in —O—Sn—O— could have one or more OH, O—, or a hydrate groups coordinated to it. In some embodiments, M^{c} and M^{a} are independently Si, Al, or Y; M^{b} is ^{99m}Tc; each X is independently —OH, ═O, or —O—; and n is 2 or 3. In some embodiments, M^{c} and M^{a} are independently Si, Al, or Y; M^{b} is ^{99m}Tc; at least an instance of Ra is —O—Sn—O—; each X is independently —OH, ═O, or —O—; and n is 2 or 3. In some embodiments, M^{c} is Al; M^{a} is Si; M^{b} is ^{99m}Tc; each X is independently —OH or ═O; and n is 2 or 3. In some embodiments, M^{c} is Al; M^{a} is Si; M^{b} is ^{99m}Tc; at least an instance of R^{a} is —O—Sn—O—, each X is independently —OH or ═O; and n is 2 or 3. In some embodiments, M^{b} is ^{99m}Tc; at least an instance of R^{a} is —O—Sn—O—; each X is independently —OH or ═O; and n is 2 or 3. In some embodiments, M^{b} is ^{99m}Tc; an instance of R^{a} is —O—Sn—O—, an instance of R^{a} is —O— or —OH—; each X is independently —OH or ═O; and n is 2 or 3. In some embodiments, the substrate may comprise a precursor of a therapeutic isotope that may be activated either before or after functionalization with the therapeutic radioisotope. For example, Lu, I, Sr, Sm, Ra, At, Ac, Th, Bi, and/or Pb may be activated (e.g., by neutron capture) to provide ¹⁷⁷Lu, ¹³¹, ⁸⁹Sr, ¹⁵³m, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

In some embodiments, the particle (e.g., nanoparticle, microparticle, or combinations thereof) comprises Formula (XI):

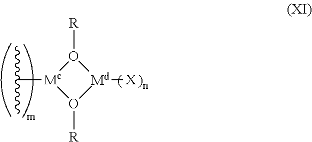

where the substrate comprises M^{c}, wherein M^{c} is selected from the group consisting of Pb, Al, Si, Y, Mn, Ga, Fe, Ti, Lu, Y, I, Sr, Sm, Ra, At, Ac, Th, and Bi; m is an integer selected from 1, 2, or 3; M^{d} is the at least one therapeutic radioisotope, wherein M^{d} is selected from ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, ²¹²Pb, and/or combinations thereof; each instance of R is —H; X is —OH; and n is an integer selected from 0, 1, 2, 3, or 4. In some embodiments, M^{c} is selected from Al, Si, and Y; and M^{d} is selected from ¹⁷⁷Lu, ⁹⁰Y, ¹³¹I, ⁸⁹Sr, ¹⁵³m, ¹⁴⁹Tb, ²²³Ra, ²²⁴Ra, ²¹¹At, ²²⁵Ac, ²²⁷Th, ²¹²Bi, ²¹³Bi, and ²¹²Pb. In some embodiments, M^{c} is selected from Al, Si, and Y; and M^{d} is ¹⁷⁷Lu. In some embodiments, M^{c} is selected from Al, Si, and Y; and M^{d} is ¹⁷⁷Lu; and X is OH.

In some embodiments, the at least one therapeutic radioisotope is distributed throughout the substrate of the particle (e.g., nanoparticle, microparticle, or combinations thereof). In some embodiments, the at least one therapeutic radioisotope distributed throughout the microsphere is ¹⁷⁷Lu, ¹³¹, ⁸⁹Sr, ¹⁵³m, ¹⁴⁹Tb, ²²³Ra, ²²⁴Ra, ²¹¹At, ²²⁵Ac, ²²⁷Th, ²¹²Bi, ²¹³Bi, and/or ²¹²Pb. In some embodiments, the therapeutic radioisotope is not bound to the surface of the substrate. In some embodiments, the therapeutic radioisotope is embedded within the substrate of the particle.

In some embodiments, the substrate comprises at least one non-metal, a metalloid, or a metal. In some embodiments, the substrate comprises a ceramic material. In some embodiments, the ceramic comprises at least one element selected from silicon, yttrium, manganese, aluminum, gallium, strontium, and titanium. In some embodiments, the substrate comprises glass. In some embodiments, the substrate comprises silicon dioxide and at least one other element selected from manganese, aluminum, gallium, yttrium, boron, strontium, and titanium. In some embodiments, the substrate comprises SiO₂, Y₂O₃, MnO₂, AlO₃, Ga₂O₃, Fe₂O₃, TiO₂, SrO₂, or combinations thereof. In some embodiments, the substrate comprises SiO₂ and at least one of Y₂O₃, MnO₂, AlO₃, Ga₂O₃, Fe₂O₃, SrO₂, and TiO₂. In some embodiments, the substrate comprises a yttrium aluminum silicon oxide.

The inorganic substrate, ceramic or glass may comprise or be an yttrium aluminum silicon oxide. Yttrium aluminum silicon oxide are described, for example, in U.S. Pat. No. 4,789,501, which is hereby incorporated by reference in its entirety.

In some embodiments, the therapeutic radioisotopic microsphere has a diameter of between 5 μm and 1000 μm.

In some embodiments, the substrate is non-porous. In some embodiments, the substrate is porous.

Some embodiments pertain to a therapeutic radioisotopic particle, comprising: at least one therapeutic radioisotope; and a substrate comprising an inorganic material that comprises metalloid or metal atoms bonded to non-metal atoms, the substrate comprising: a core extending to a surface, the core comprising a first portion of the metalloid and/or metal atoms bonded to the non-metal atoms and the surface comprising a second portion of the metalloid and/or metal atoms bonded to the non-metal atoms. In some embodiments, the therapeutic radioisotope is bound directly to the substrate through non-metal atoms of the surface of the substrate and/or wherein the therapeutic radioisotope is bound to the substrate through an inorganic bridge comprising non-metal atoms of the surface of the substrate.

In some embodiments, the therapeutic radioisotope is bound directly to the substrate through non-metal atoms of the surface of the substrate. In some embodiments, the substrate is bound to the substrate through an inorganic bridge through non-metal atoms of the surface of the substrate.

In some embodiments, the substrate comprises a substantially homogeneous mixture of constituent chemical elements. In some embodiments, the surface comprises at least a portion of the constituent chemical elements.

In some embodiments, the non-metal atoms are oxygen atoms. In some embodiments, at least a portion of the oxygen atoms at the surface of the substrate are hydroxyl groups.

Some embodiments pertain to a therapeutic radioisotopic particle, comprising: an inorganic substrate with a surface and at least one therapeutic radioisotope, wherein the substrate comprises at least one non-metal and at least a metalloid and/or a metal. In some embodiments, the therapeutic radioisotope is bound to the surface of the substrate by a Lewis acid-base coordination bond to an inorganic Lewis base.

Some embodiments pertain to a therapeutic radioisotopic particle, comprising: an inorganic substrate having a surface; and at least one therapeutic radioisotope; wherein the substrate comprises at least one non-metal and at least a metalloid and/or a metal. In some embodiments, the therapeutic radioisotope is bound to the surface of the substrate by a chemical bond to an oxygen of an inorganic species.

Some embodiments pertain to a therapeutic radioisotopic particle, comprising: an inorganic substrate comprising a surface having one or more electron donating functionalities; and at least one therapeutic radioisotope. In some embodiments, the therapeutic radioisotope is bound directly to the surface and/or is bound to the surface through an inorganic bridge during preparation of the therapeutic radioisotopic particle via chemical coupling with the one or more electron donating functionalities.

In some embodiments, the therapeutic radioisotope is bound directly to the surface of the substrate. In some embodiments, the substrate comprises a metal oxide, a transition metal oxide, a metalloid oxide, or combinations thereof. In some embodiments, the therapeutic radioisotope is bound to the substrate via a chemical bond selected from an ionic bond, a covalent bond, or a coordinate bond. In some embodiments, the therapeutic radioisotope is bound via a coordinate bond.

Some embodiments pertain to a therapeutic radioisotopic particle, comprising: a ceramic particle substrate and at least one therapeutic radioisotope; wherein the therapeutic radioisotope is coupled to the surface of the ceramic particle substrate as a Lewis acid-base adduct of an inorganic Lewis base.

In some embodiments, the inorganic Lewis base is a component of the substrate and the therapeutic radioisotope is directly coupled to the substrate surface through the inorganic Lewis base.

In some embodiments, the therapeutic radioisotope is coupled to the surface of the ceramic microsphere substrate through an inorganic linker comprising the Lewis base.

In some embodiments, the inorganic linker is a metal oxide. In some embodiments, the metal oxide is a tin oxide.

In some embodiments, the Lewis base is an oxygen of a metal oxide or metalloid oxide. In some embodiments, the Lewis base is the oxygen of a tin oxide.

In some embodiments, the at least one therapeutic radioisotope is an alpha emitter, a beta emitter, or a positron emitter. In some embodiments, the at least one therapeutic radioisotope is a metallic radioisotope. In some embodiments, the therapeutic radioisotope is bound directly to the substrate at the surface. In some embodiments, the at least one therapeutic radioisotope is ¹⁷⁷Lu. In some embodiments, the at least one therapeutic radioisotope comprises one or more of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

In some embodiments, the therapeutic radioisotopic microsphere comprises Formula (XI):

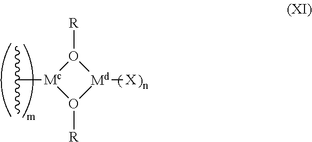

where the substrate comprises M^{c}, wherein M^{c} is selected from Pb, Al, Si, Y, Mn, Ga, Fe, Ti, Lu, Y, I, Sr, Sm, Ra, At, Ac, Th, and Bi; m is an integer selected from 1, 2, or 3; M^{d} is the at least one therapeutic radioisotope, wherein M^{d} is selected from ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, ²¹²Pb, and/or combinations thereof; each instance of R is —H; X is —OH; and n is an integer selected from 0, 1, 2, 3, or 4. In some embodiments, M^{c} is selected from Al, Si, and Y; and M^{d} is ¹⁷⁷Lu.

In some embodiments, a second therapeutic radioisotope is distributed throughout the substrate of the microsphere. In some embodiments, the second therapeutic radioisotope is not bound to the surface of the substrate.

In some embodiments, the substrate comprises at least one non-metal and a metalloid, a transition metal, and a metal. In some embodiments, the substrate comprises a ceramic material. In some embodiments, the ceramic comprises at least one element selected from silicon, yttrium, manganese, aluminium, gallium, and titanium. In some embodiments, the substrate comprises glass. In some embodiments, the substrate comprises silicon dioxide and at least one other element selected from manganese, aluminium, gallium, yttrium, boron and titanium. In some embodiments, the substrate comprises SiO₂, Y₂O₃, MnO₂, AlO₃, Ga₂O₃, Fe₂O₃, TiO₂, SrO₂, or combinations thereof. In some embodiments, the substrate comprises SiO₂ and at least one of Y₂O₃, MnO₂, AlO₃, Ga₂O₃, Fe₂O₃, SrO₂, and TiO₂. In some embodiments, the substrate comprises an yttrium aluminum silicon oxide.

In some embodiments, the particle has a diameter of between 5 μm and 1000 μm. In some embodiments, the particle has a diameter of between 10 nm and 1000 nm.

In some embodiments, the substrate is non-porous. In some embodiments, the substrate is porous.

Some embodiments pertain to a particle made by a method comprising: providing the substrate; chemically coupling the at least one therapeutic radioisotope to the substrate to provide the particle.

Some embodiments pertain to a particle made by a method comprising: providing a substrate comprising: an inorganic material comprising metal or metalloid atoms bonded to non-metal atoms; a core comprising a first portion of the non-metal atoms; and a surface comprising a second portion of the non-metal atoms; providing at least one therapeutic radioisotope; and chemically coupling the at least one therapeutic radioisotope to the surface layer of the substrate through the second portion of non-metal atoms to provide the therapeutic radioisotopic particle.

In some embodiments, the substrate comprises an embedded therapeutic radioisotope. In some embodiments, the substrate comprises a precursor of an embedded therapeutic radioisotope. In some embodiments, the embedded therapeutic radioisotope is the therapeutic radioisotope. In some embodiments, the precursor of the embedded therapeutic radioisotope is activated by neutron bombardment to provide an embedded therapeutic radioisotope.

In some embodiments, the method comprises providing the at least one therapeutic radioisotope as a salt prior to chemically coupling the at least one therapeutic radioisotope to the surface layer of the inorganic substrate. In some embodiments, the salt is an alkali metal salt, an alkali earth metal salt, a halogen salt, a polyatomic salt, or a salt with an organic acid. In some embodiments, the chemical functionalization is carried out in the presence of a reducing agent. In some embodiments, the reducing agent is selected from one or more of a stannous salt, a stannous hydrate, concentrated HCl, sodium borohydride, sodium diothionite, ferrous sulfate, ferric chloride plus ascorbic acid, hypophosphorus acid, and/or hydrazine.

In some embodiments, the therapeutic radioisotope is ^{99m}Tc and the chemical functionalization is carried out in the presence of a tin salt. In some embodiments, the therapeutic radioisotope is provided in the form of ^{99m}Tc pertechnetate and the chemical functionalization is carried out in the presence of stannous ions.

In some embodiments, the therapeutic radioisotope is ⁸⁹Zr. In some embodiments, the ⁸⁹Zr is provided in the form of 89Zr oxalate. In some embodiments, the substrate comprises a precursor of the therapeutic radioisotope and/or the surface of the substrate comprises a precursor to the therapeutic radioisotope.

In some embodiments, the method includes exposing the precursor to neutron bombardment to prepare a radioactive radioisotope.

Some embodiments pertain to a method for preparing a particle, comprising: providing a ceramic particle substrate; and reacting the ceramic particle substrate with a therapeutic radioisotope, and/or a precursor of the therapeutic radioisotope, under conditions suitable to couple the therapeutic radioisotope, and/or the precursor of the therapeutic radioisotope to the surface of the ceramic particle. In some embodiments, the substrate comprises an embedded precursor of a therapeutic radioisotope or an embedded therapeutic radioisotope.

In some embodiments, the method includes reacting the ceramic particle substrate with a therapeutic radioisotope or a precursor of a therapeutic radioisotope under conditions suitable to couple the precursor of the therapeutic radioisotope or the therapeutic radioisotope to the surface of the ceramic particle.

In some embodiments, the radioisotope is coupled to the surface of the ceramic particle in the form of a Lewis acid-base adduct. In some embodiments, the therapeutic radioisotope is a metallic radio isotope. In some embodiments, the therapeutic radioisotope is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, Al¹⁸F and/or combinations thereof. In some embodiments, the therapeutic radioisotope is provided in the form of a salt. In some embodiments, the therapeutic radioisotope and/or the precursor thereof is reacted with the ceramic particle in the presence of a reducing agent. In some embodiments, the reducing agent is selected from one or more of a stannous salt, a stannous hydrate, HCl, sodium borohydride, sodium diothionite, ferrous sulfate, ferric chloride plus ascorbic acid, hypophosphorus acid, and/or hydrazine. In some embodiments, the therapeutic radioisotope is ^{99m}Tc. In some embodiments, the ^{99m}Tc is provided in the form of a pertechnetate salt. In some embodiments, the ^{99m}Tc is provided in the form of a pertechnetate salt and the reaction is carried out in the presence of stannous ions. In some embodiments, the therapeutic radioisotope is ⁸⁹Zr. In some embodiments, the ⁸⁹Zr is provided in the form of ⁸⁹Zr oxalate. In some embodiments, the reaction is carried out in the presence of a base.

In some embodiments, the method includes exposing the particle to neutron bombardment to convert a precursor of therapeutic radioisotope to the therapeutic radioisotope.

Some embodiments pertain to a for making the particle as disclosed herein, the method comprising: providing the inorganic substrate; and chemically functionalizing the inorganic substrate with a precursor of a precursor of the therapeutic radioisotope and/or the therapeutic radioisotope to provide a precursor of the particle or the particle.

In some embodiments, the method includes chemically functionalizing the substrate with a precursor of the at least one therapeutic radioisotope or the at least one therapeutic radioisotope.

In some embodiments, the precursor of therapeutic radioisotope or the therapeutic radioisotope is provided in the form of a salt. In some embodiments, the salt is an alkali metal salt, an alkali earth metal salt, a halogen salt, a polyatomic salt, or a salt with an organic acid.

In some embodiments, the method includes adding a reducing agent during the chemical functionalization step. In some embodiments, the reducing agent is selected from one or more of a stannous salt, a stannous hydrate, HCl, sodium borohydride, sodium diothionite, ferrous sulfate, ferric chloride plus ascorbic acid, hypophosphorus acid, and/or hydrazine. In some embodiments, the radioisotope is ^{99m}Tc. In some embodiments, the ^{99m}Tc is provided in the form of a pertechnetate salt. In some embodiments, the ^{99m}Tc is provided in the form of a pertechnetate salt and the reaction is carried out in the presence of stannous ions. In some embodiments, the radioisotope is ⁸⁹Zr. In some embodiments, the ⁸⁹Zr is provided in the form of ⁸⁹Zr oxalate.

In some embodiments, the reaction is carried out in the presence of a base. In some embodiments, the reaction is carried out in aqueous conditions.

In some embodiments, the method additionally comprises recovering the particle and/or washing the particle to remove unreacted radioisotope. In some embodiments, the method additionally comprises re-suspending the particle in a pharmaceutically acceptable injectable aqueous medium.

Some embodiments pertain to a particle obtainable by a method according to any of the above methods.

Some embodiments pertain to a method for treating a patient. In some embodiments, the method comprises introducing the population of therapeutic radioisotopic particles to the patient. In some embodiments, the method comprises allowing the population of therapeutic radioisotopic particles to distribute within the patient over a period of time.

In some embodiments, the target site for treatment in the patient is the liver. In some embodiments, the target site for treatment is a tumor of the liver of the patient.

Some embodiments pertain to a method for treating liver cancer in a patient. In some embodiments, the method comprises providing a population of therapeutic radioisotopic particles. In some embodiments, the method comprises delivering the population of therapeutic radioisotopic particles to the patient by introducing the population of therapeutic radioisotopic particles to a first position in a vasculature of a body of the patient. In some embodiments, the method comprises allowing the population of therapeutic radioisotopic particles to distribute within the body of the patient.

In some embodiments, the therapeutic radioisotopic particles comprise: a substrate comprising a core extending to a surface; and at least one therapeutic radioisotope chemically bound to the surface of the substrate.

In some embodiments of the methods disclosed herein, the therapeutic radioisotopic particles comprise: a substrate comprising a core extending to a surface; and a therapeutic radioisotope chemically bound to the surface of the substrate.

In some embodiments, the therapeutic radioisotopic particles used in the methods disclosed herein are the therapeutic radioisotopic particles of any of the above embodiments. In some embodiments, the population of therapeutic radioisotopic particles comprises one or more of a therapeutic radioisotopic particle of any of the above embodiments and as disclosed elsewhere herein.

In some embodiments, the therapeutic radioisotopic particle comprises: at least one therapeutic radioisotope; and a substrate comprising an inorganic material that comprises metalloid or metal atoms bonded to non-metal atoms, the substrate comprising: a core extending to a surface, the core comprising a first portion of the metalloid or metal atoms bonded to the non-metal atoms and the surface comprising a second portion of the metalloid or metal atoms bonded to the non-metal atoms; wherein the therapeutic radioisotope is bound directly to the substrate through non-metal atoms of the surface of the substrate and/or wherein the therapeutic radioisotope is bound to the substrate through an inorganic bridge comprising non-metal atoms of the surface of the substrate.

In some embodiments, the therapeutic radioisotopic particle comprises: an inorganic substrate with a surface; and at least one therapeutic radioisotope; wherein the substrate comprises at least one non-metal and at least a metalloid or a metal; and wherein the therapeutic radioisotope is bound to the surface of the substrate by a Lewis acid-base coordination bond to an inorganic Lewis base.

In some embodiments, the therapeutic radioisotopic particle comprises: an inorganic substrate having a surface; and at least one therapeutic radioisotope; wherein the substrate comprises at least one non-metal and at least a metalloid or a metal; and wherein the therapeutic radioisotope is bound to the surface of the substrate by a chemical bond to an oxygen of an inorganic species.

In some embodiments, the therapeutic radioisotopic particle comprises: an inorganic substrate comprising a surface having one or more electron donating functionalities; and at least one therapeutic radioisotope; wherein the therapeutic radioisotope is bound directly to the surface and/or is bound to the surface through an inorganic bridge during preparation of the therapeutic microsphere via chemical coupling with the one or more electron donating functionalities.

In some embodiments, the therapeutic radioisotopic particle comprises: a ceramic microsphere substrate and at least one therapeutic radioisotope; wherein the therapeutic radioisotope is coupled to the surface of the ceramic microsphere substrate as a Lewis acid-base adduct of an inorganic Lewis base.

Some embodiments pertain to a kit. In some embodiments, the kit comprises a particle comprising: a substrate comprising an inorganic material that comprises metalloid or metal atoms bonded to non-metal atoms. In some embodiments, the substrate comprises a core extending to a surface, the core comprising a first portion of the metalloid or metal atoms bonded to the non-metal atoms and the surface comprising a second portion of the metalloid or metal atoms bonded to the non-metal atoms. In some embodiments, the kit comprises instructions for reacting a therapeutic radioisotope with the substrate such as to bind the radioisotope directly to the substrate through at least a portion of the non-metal atoms at the surface of the substrate.

Some embodiments pertain to a kit comprising a particle comprising: a substrate having at least one non-metal, a metalloid, or a transition metal oxide; and instructions for binding a therapeutic radioisotope to the surface of the substrate through a Lewis acid-base coordination bond.

Some embodiments pertain to a kit comprising a particle comprising a ceramic particle substrate and instructions for carrying out a reaction in which a therapeutic radioisotope is coupled to the ceramic particle substrate as a Lewis acid base adduct.

In some embodiments, the kit comprises 50 μl to 2 ml of particles by packed volume, in a sealed unit. In some embodiments, the particles are provided in a vial or a syringe.

In some embodiments, the kit comprises instructions for carrying out a reaction in which a therapeutic radioisotope is coupled to the substrate as a Lewis acid base adduct. In some embodiments, the kit comprises instructions for activating a precursor to a therapeutic isotope within the substrate to provide a therapeutic isotope. In some embodiments, the instructions for activating the precursor to the therapeutic isotope within the substrate indicate that the precursor to the therapeutic isotope is activated prior to coupling the therapeutic radio isotope to the substrate.

In some embodiments, the kit comprises an therapeutic radioisotope is selected from ⁹⁹mTc, ²⁰¹Th ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, Al¹⁸F, ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, ²¹²Pb, or combinations thereof.

In some embodiments, the kit additionally comprises a reducing agent. In some embodiments, the reducing agent is selected from one or more of a stannous salt, concentrated HCl, sodium borohydride, sodium diothionite, ferrous sulfate, ferric chloride plus ascorbic acid, hypophosphorus acid, and/or hydrazine.

In some embodiments, the substrate comprises a yttrium oxide aluminosilicate.

In some embodiments, the kit further comprises one or more of a vascular access needle, a vascular guidewire, a vascular sheath (e.g., 4-6Fr), a vascular catheter (4-5Fr), a microcatheter, syringes, and a vial.

Some embodiments pertain to a population of particles comprising at least one therapeutic particle. In some embodiments, the at least one therapeutic particle comprises at least one therapeutic radioisotope. In some embodiments, the population comprise particles having a substrate comprising an inorganic material that comprises metalloid or metal atoms bonded to non-metal atoms, the substrate comprising: a core extending to a surface, the core comprising a first portion of the metalloid and/or metal atoms bonded to the non-metal atoms and the surface comprising a second portion of the metalloid and/or metal atoms bonded to the non-metal atoms. In some embodiments, the radioisotope is bound directly to the substrate through non-metal atoms of the surface of the substrate and/or the radioisotope is bound to the substrate through an inorganic bridge comprising non-metal atoms of the surface of the substrate. In some embodiments, the therapeutic radioisotope is bound directly to the substrate through non-metal atoms of the surface of the substrate and/or the radioisotope is bound to the substrate through an inorganic bridge comprising non-metal atoms of the surface of the substrate. In some embodiments, the therapeutic radioisotope and/or the radioisotope is bound directly to the substrate through non-metal atoms of the surface of the substrate. In some embodiments, the therapeutic radioisotope and/or the radioisotope substrate is bound to the substrate through an inorganic bridge through non-metal atoms of the surface of the substrate. In some embodiments, the substrate comprises a substantially homogeneous mixture of constituent chemical elements. In some embodiments, the surface comprises at least a portion of the constituent chemical elements. In some embodiments, the non-metal atoms are oxygen atoms. In some embodiments, at least a portion of the oxygen atoms at the surface of the substrate are hydroxyl groups.

Some embodiments pertain to a population of particles, a particle of the population comprising: an inorganic substrate with a surface. In some embodiments, the population comprises at least one therapeutic radioisotope. In some embodiments, the substrate comprises at least one non-metal and at least a metalloid and/or a metal. In some embodiments, the therapeutic radioisotope is bound to the surface of the substrate by a Lewis acid-base coordination bond to an inorganic Lewis base.

Some embodiments pertain to a population of particles, a particle of the population comprising an inorganic substrate having a surface. In some embodiments, the population comprises at least one therapeutic radioisotope. In some embodiments, the substrate comprises at least one non-metal and at least a metalloid and/or a metal. In some embodiments, the therapeutic radioisotope is bound to the surface of the substrate by a chemical bond to an oxygen of an inorganic species.

Some embodiments pertain to a population of particles, a particle of the population comprising an inorganic substrate comprising a surface having one or more electron donating functionalities. In some embodiments, the population comprises at least one therapeutic radioisotope. In some embodiments, the therapeutic radioisotope is bound directly to the surface and/or is bound to the surface through an inorganic bridge during preparation of the population via chemical coupling with the one or more electron donating functionalities.

In some embodiments, the therapeutic radioisotope is bound directly to the surface of the substrate.

In some embodiments, the substrate comprises a metal oxide, a transition metal oxide, a metalloid oxide, or combinations thereof.

In some embodiments, the therapeutic radioisotope is bound to the substrate via a chemical bond selected from an ionic bond, a covalent bond, or a coordinate bond. In some embodiments, the radioisotope is bound via a coordinate bond.

Some embodiments pertain to a population, comprising a ceramic particle substrate. In some embodiments, the ceramic particle comprises at least one therapeutic radioisotope.

In some embodiments, the radioisotope is coupled to the surface of the ceramic particle substrate as a Lewis acid-base adduct of an inorganic Lewis base. In some embodiments, the inorganic Lewis base is a component of the substrate and the isotope is directly coupled to the substrate surface through the inorganic Lewis base. In some embodiments, the radioisotope is coupled to the surface of the ceramic microsphere substrate through an inorganic linker comprising the Lewis base. In some embodiments, the inorganic linker is a metal oxide. In some embodiments, the metal oxide is a tin oxide.

In some embodiments, the Lewis base is an oxygen of a metal oxide or metalloid oxide. In some embodiments, the Lewis base is the oxygen of a tin oxide.

In some embodiments, the therapeutic radioisotope is bound directly to the substrate at the surface. In some embodiments, the at least one therapeutic radioisotope is ¹⁷⁷Lu. In some embodiments, the at least one therapeutic radioisotope comprises one or more of ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, ¹⁷⁷Lu, Al¹⁸F, ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb. In some embodiments, the at least one therapeutic radioisotope is ¹⁷⁷Lu.

In some embodiments, a particle of the population comprises a structure of Formula (V):

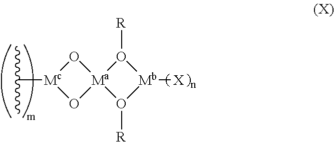

where the substrate comprises M^{c} and M^{c} is selected from Pb, Al, Si, Y, Mn, Ga, Fe, and Ti; m is an integer selected from 1, 2, or 3; M^{b} is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, ¹⁷⁷Lu, Al¹⁸F, and/or combinations thereof; M^{a} is either an atom of the substrate or a bridging metal atom and M^{a} is selected from Sn, Pb, Al, Si, Y, Mn, Ga, Fe, and Ti; each instance of R is not present or is —H; X is selected from —OH, ═O, and —O; and n is an integer selected from 0, 1, 2, 3, or 4. In some embodiments, M^{c} is Al; the substrate comprises M^{a} and M^{a} is Si; M^{b} is ⁸⁹Zr; each X is independently —OH or —O⁻; and n is 1 or 2. In some embodiments, M^{b} is ⁸⁹Zr, X is —OH, and n is 2. In some embodiments, M^{c} is Si; M^{a} is Sn; M^{b} is ^{99m}Tc; each X is independently —OH or —O—; and n is 2 or 3. In some embodiments, M^{b} is ^{99m}Tc, X is —OH, and n is 3.

In some embodiments, a particle of the population comprises a structure of Formula (VIII):

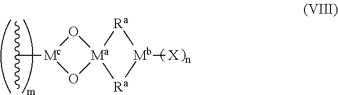

where the substrate comprises M^{a} and M^{c} and where M^{a} and M^{c} are independently selected from Pb, Al, Si, Y, Mn, Ga, Fe, and Ti; m is an integer selected from 1, 2, or 3; M^{b} is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁷⁷Lu, Al¹⁸F, and/or combinations thereof; each instance of R^{a} is independently OH, O, or —O—Sn(X)ₙ—O—; X is selected from —OH, =═O, and —O—; and n is an integer selected from 0, 1, 2, 3, or 4. In some embodiments, M^{c} is Al; M^{a} is Si; M^{b} is ^{99m}Tc; each X is independently —OH or ═O; and n is 2 or 3. In some embodiments, M^{b} is ^{99m}Tc; at least an instance of R^{a} is —O—Sn(X)ₙ—O—, each X is independently —OH or ═O; and n is 2 or 3. In some embodiments, M^{b} is ^{99m}Tc; an instance of R^{a} is —O—Sn—O—; an instance of R^{a} is —O— or —OH—; each X is independently —OH or ═O; and n is 2 or 3.

In some embodiments, a particle of the population comprises a structure of Formula (XI):

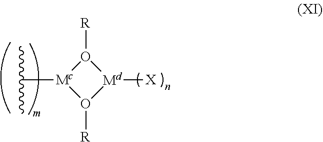

where the substrate comprises M^{c}, wherein M^{c} is selected from Pb, Al, Si, Y, Mn, Ga, Fe, Ti, Lu, Y, I, Sr, Sm, Ra, At, Ac, Th, and Bi; m is an integer selected from 1, 2, or 3; M^{d} is the at least one therapeutic radioisotope, wherein M^{d} is selected from ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, ²¹²Pb, and/or combinations thereof; each instance of R is —H; X is —OH; and n is an integer selected from 0, 1, 2, 3, or 4. In some embodiments, M^{c} is selected from Al, Si, and Y; and M^{d} is ¹⁷⁷Lu.

In some embodiments, a particle of the population comprises one or more of Formula (V), (VIII), and (XI).

In some embodiments of a particle of the population, the at least one therapeutic radioisotope is distributed throughout the substrate of the microsphere. In some embodiments of a particle of the population, the therapeutic radioisotope is not bound to the surface of the substrate. In some embodiments of a particle of the population, the at least one therapeutic radioisotope is not distributed throughout the substrate of the microsphere. In some embodiments of a particle of the population, the therapeutic radioisotope is bound to the surface of the substrate.

In some embodiments of a particle of the population, the substrate comprises at least one non-metal and a metalloid, a transition metal, and a metal. In some embodiments, substrate comprises a ceramic material. In some embodiments, ceramic comprises at least one element selected from silicon, yttrium, manganese, aluminium, gallium, and titanium. In some embodiments, the substrate comprises glass. In some embodiments, the substrate comprises silicon dioxide and at least one other element selected from manganese, aluminium, gallium, yttrium, boron and titanium. In some embodiments, the substrate comprises SiO₂, SrO₂, Y₂O₃, MnO₂, AlO₃, Ga₂O₃, Fe₂O₃, TiO₂, or combinations thereof. In some embodiments, the substrate comprises SiO₂ and at least one of SrO₂, Y₂O₃, MnO₂, AlO₃, Ga₂O₃, Fe₂O₃, and TiO₂. In some embodiments, the substrate comprises an yttrium aluminum silicon oxide. In some embodiments, the particle has a diameter of between 5 μm and 1000 μm. In some embodiments, the particle has a diameter of between 10 nm and 1000 nm. In some embodiments, the substrate is non-porous. In some embodiments, the substrate is porous.

Any of the methods described above, or described elsewhere herein, can include one or more of the following features.

In some embodiments, the substrate comprises the at least one therapeutic radioisotope. In some embodiments, the substrate comprises a precursor of the at least one therapeutic radioisotope. In some embodiments, the precursor of the at least one therapeutic radioisotope is activated by neutron bombardment to provide the at least one therapeutic radioisotope.

In some embodiments, the at least one therapeutic radioisotope is provided as a salt prior to chemically coupling the at least one therapeutic radioisotope to the surface layer of the inorganic substrate. In some embodiments, the at least one therapeutic radioisotope is chemically coupled to the surface layer of the substrate. In some embodiments, the at least one therapeutic radioisotope as a salt prior to chemically coupling the at least one therapeutic radioisotope to the surface layer of the inorganic substrate. In some embodiments, the salt is an alkali metal salt, an alkali earth metal salt, a halogen salt, a polyatomic salt, or a salt with an organic acid.

In some embodiments, the chemical coupling is carried out in the presence of a reducing agent. In some embodiments, the reducing agent is selected from one or more of a stannous salt, HCl, sodium borohydride, sodium diothionite, ferrous sulfate, ferric chloride plus ascorbic acid, hypophosphorus acid, and/or hydrazine. In some embodiments, the reducing agent is a tin salt (e.g., a stannous salt such as stannous chloride, in order to provide stannous ions) or a tin hydrate (e.g., stannous hydrate). In some embodiments, the therapeutic radioisotope is ^{99m}Tc and the chemical functionalization is carried out in the presence of a tin salt. In some embodiments, the therapeutic radioisotope is provided in the form of ^{99m}Tc pertechnetate and the chemical coupling is carried out in the presence of stannous ions.

In some embodiments, the therapeutic radioisotope is ⁸⁹Zr. In some embodiments, the therapeutic radioisotope is ⁸⁹Zr and is provided in the form of ⁸⁹Zr oxalate.

In some embodiments, the substrate comprises a precursor of the therapeutic radioisotope.

In some embodiments, the method further comprises exposing the precursor to neutron bombardment to prepare the therapeutic radioisotope.

Some embodiments pertain to a method for preparing a therapeutic radioisotopic microsphere. In some embodiments, a ceramic microsphere substrate comprising a precursor of a therapeutic radioisotope or a therapeutic radioisotope is provided. In some embodiments, the ceramic microsphere substrate is reacted with a therapeutic radioisotope or a precursor of the therapeutic radioisotope under conditions suitable to couple the precursor of the therapeutic radioisotope or the therapeutic radioisotope to the surface of the ceramic microsphere.

The radioisotope may be reacted with the microsphere in the presence of a base. The base may be, for example, selected from alkali or alkali earth metal carbonates (such as sodium carbonate and calcium carbonate), alkali hydroxides, or the like. In some embodiments, the base may be selected from NaOH, KOH, or the like. In some embodiments, the base is a weak base. In some embodiments, the base is an inorganic base. In some embodiments the radioisotope may be ⁸⁹Zr, for example provided as salt, such as ⁸⁹Zirconium oxalate or a halide salt such as ⁸⁹Zirconium chloride.

In some embodiments, the precursor of therapeutic radioisotope, the therapeutic radioisotope, the precursor of the radioisotope, and/or the radioisotope is reacted with the substrate in the presence of a reducing agent. In some embodiments, the reducing agent is selected from one or more of a stannous salt, HCl, sodium borohydride, sodium diothionite, ferrous sulfate, ferric chloride plus ascorbic acid, hypophosphorus acid, and/or hydrazine.

In some embodiments, the radioisotope is ⁹⁹mTc. In some embodiments, the ⁹⁹mTc is provided in the form of a pertechnetate salt. In some embodiments, the ⁹⁹mTc is provided in the form of a pertechnetate salt and the reaction is carried out in the presence of stannous ions.

In some embodiments, the radioisotope is ⁸⁹Zr. In some embodiments, the ⁸⁹Zr is provided in the form of ⁸⁹Zr oxalate.

In some embodiments, the substrate is exposed to neutron bombardment to convert the precursor of therapeutic radioisotope to the therapeutic radioisotope.

In some embodiments, the reaction is carried out in the presence of a base. In some embodiments, the reaction is carried out in aqueous conditions.

In some embodiments, the therapeutic radioisotopic microsphere is recovered by washing the therapeutic radioisotopic microsphere to remove unreacted radioisotope. In some embodiments, the therapeutic radioisotopic microsphere is resuspended in a pharmaceutically acceptable injectable aqueous medium.

In some embodiments, the substrate comprises a precursor of the therapeutic radioisotope and/or the therapeutic radioisotope.

Some embodiments pertain to an therapeutic microsphere obtainable by a method as described above or elsewhere herein. In some embodiments, a distribution of therapeutic radioisotopic microspheres in a patient is determined. In some embodiments, therapeutic radioisotopic microspheres are introduced to the patient. In some embodiments, the therapeutic radioisotopic microspheres are allowed to distribute within the patient over a period of time. In some embodiments, the therapeutic radioisotopic microspheres comprise a substrate comprising a core extending to a surface and a therapeutic radioisotope chemically bound to the surface of the substrate.

Some embodiments pertain to a method of determining a dose of radiation from therapeutic radioisotopic microspheres at a target site for treatment in a patient. In some embodiments, therapeutic radioisotopic microspheres are introduced to the patient. In some embodiments, the therapeutic radioisotopic microspheres are allowed to distribute within the patient over a period of time. In some embodiments, the therapeutic radioisotopic microspheres comprise a substrate comprising a core extending to a surface and a therapeutic radioisotope chemically bound to the surface of the substrate.

Some embodiments pertain to a method of treating cancer (e.g., malignant tumors) or benign tumors (non-malignant tumors) in a patient in need of treatment. Some embodiments pertain to a method of treating vascularized tumors having a vascular supply (e.g., malignant or benign tumors), such as those found in liver cancer (e.g., hepatic neoplasias such as hepatocellular carcinoma —HCC, as well as tumors derived from metastasis of other tumors to the liver, such as neuroendocrine tumors and colorectal tumors), as well as those of the brain, prostate, lung, spleen and kidney, for example. In some embodiments, a population of therapeutic microspheres is introduced to the patient. In some embodiments, the therapeutic microspheres are allowed to distribute within the patient over a period of time.

In some embodiments, the therapeutic microspheres distribute in the body soon after injection (e.g., in a matter of minutes). In some embodiments, the therapeutic microspheres distribute within the patient (e.g., in target and or off-target areas) within a period of equal to or less than about: 2 minutes, 5 minutes, 10 minutes, 30 minutes, or ranges including and/or spanning the aforementioned values.

In some embodiments, the target site for treatment in the patient is the liver. In some embodiments, the target site for treatment is a tumor of the liver of the patient.

Some embodiments pertain to a method for treating liver cancer in a patient. In some embodiments, a population of therapeutic radioisotopic microspheres is provided. In some embodiments, the population of therapeutic radioisotopic microspheres is delivered to the patient by introducing the population of therapeutic radioisotopic microspheres to a first position in a vasculature of a body of the patient. In some embodiments, the population of therapeutic radioisotopic microspheres is allowed to distribute within the body of the patient. In some embodiments, the method includes administering to the patient the additional amount of therapeutic radioisotopic microspheres.

Some embodiments pertain to a method of reducing gastrointestinal damage during treatment of a patient in need of radioisotopic cancer treatment. In some embodiments, a first population of therapeutic radioisotopic microspheres is introduced to the patient. In some embodiments, the first population of therapeutic radioisotopic microspheres is allowed to distribute within the patient over a period of time. In some embodiments, the therapeutic radioisotopic microspheres comprise a substrate comprising a core extending to a surface, a therapeutic radioisotope chemically bound to the surface of the substrate.

Some embodiments pertain to a method of reducing lung damage during treatment of a patient in need of radioisotopic cancer treatment. In some embodiments, a first population of therapeutic radioisotopic microspheres is introduced to the patient. In some embodiments, the first population of therapeutic radioisotopic microspheres is allowed to distribute within the patient over a period of time.

Some embodiments pertain to a kit. In some embodiments, the kit comprises a microsphere comprising a substrate comprising an inorganic material that comprises metalloid or metal atoms bonded to non-metal atoms. In some embodiments, the substrate comprises a core extending to a surface, the core comprising a first portion of the metalloid or metal atoms bonded to the non-metal atoms and the surface comprising a second portion of the metalloid or metal atoms bonded to the non-metal atoms and instructions for reacting a therapeutic radioisotope with the substrate such as to bind the radioisotope directly to the substrate through at least a portion of the non-metal atoms at the surface of the substrate.

Some embodiments pertain to a kit. In some embodiments, the kit comprises a microsphere comprising a substrate; wherein the substrate comprises at least one non-metal, a metalloid, or a transition metal oxide and instructions for binding a therapeutic radioisotope to the surface of the substrate through a Lewis acid-base coordination bond.

Some embodiments pertain to a kit. In some embodiments, the kit comprises a microsphere comprising a ceramic microsphere substrate and instructions for carrying out a reaction in which a therapeutic radioisotope is coupled to the ceramic microsphere substrate as a Lewis acid base adduct.

In some embodiments, the kit comprises 50 μl to 2 ml of microspheres by packed volume, in a sealed unit. In some embodiments, the microspheres are provided in a vial or a syringe. In some embodiments, the therapeutic radioisotope is selected from ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, 44Sc, ⁵¹Cr, ¹⁸F, Al¹⁸F, and/or combinations thereof.

In some embodiments, the kit comprises instructions for carrying out a reaction in which a therapeutic radioisotope is coupled to the substrate as a Lewis acid base adduct.

In some embodiments, the kit comprises instructions for activating a precursor to a therapeutic isotope within the substrate to provide a therapeutic isotope. In some embodiments, the instructions for activating the precursor to the therapeutic isotope within the substrate indicate that the precursor to the therapeutic isotope is activated prior to coupling the therapeutic radio isotope to the substrate. In some embodiments, the therapeutic radioisotope is one or more of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb. In some embodiments, the therapeutic radioisotope is one or more of ¹⁷⁷Lu, ⁹⁰Y, ¹³¹I, ⁸⁹Sr, ¹⁵³Sm, and/or ²²³Ra. In some embodiments, the therapeutic radioisotope is ¹⁷⁷Lu. In some embodiments, the therapeutic radioisotope is ⁹⁰Y.

In some embodiments, the kit additionally comprises a reducing agent. In some embodiments, the reducing agent is selected from one or more of a stannous salt, concentrated HCl, sodium borohydride, sodium diothionite, ferrous sulfate, ferric chloride plus ascorbic acid, hypophosphorus acid, and/or hydrazine. In some embodiments, the reducing agent is a stannous salt, the therapeutic radioisotope is ⁹⁹mTc and the radioisotope is in the form of a pertechnetate salt.

In some embodiments, the therapeutic radioisotope is ⁸⁹Zr. In some embodiments, the therapeutic radioisotope is in the form of ⁸⁹Zr zirconium oxalate.

In some embodiments, the therapeutic radioisotope is ¹⁷⁷Lu.

In some embodiments, the substrate comprises a yttrium oxide aluminosilicate.

In some embodiments, the kit further comprises one or more of a vascular access needle, a vascular guidewire, a vascular sheath (e.g., 4-6Fr), a vascular catheter (4-5Fr), a microcatheter, syringes, and a vial.

### BRIEF DESCRIPTION OF THE DRAWINGS

The features of the therapeutic particles disclosed herein are described below with reference to the drawings of certain embodiments. The illustrated embodiments are intended to demonstrate, but not to limit, the present disclosure.

FIG. 1 depicts the periodic table.

FIG. 2 depicts results of a detachment study for an embodiment of an therapeutic radioisotopic particle.

FIG. 3A depicts results for a stability study for an embodiment of a therapeutic radioisotopic particle with a ⁹⁹mTc microsphere.

FIG. 3B depicts results for a stability study for an embodiment of a therapeutic radioisotopic particle with a ⁸⁹Zr microsphere.

FIG. 4A depicts results for a stability study for a comparator particle comprising macro-aggregated albumen labelled with technetium-99m.

FIG. 4B provides results of radioisotope functionalization performed at varying pH levels and in various buffers.

FIGS. 5A and 5B provide axial images from woodchuck with large bilateral hepatoma. FIG. 5A was performed using T2-weighted MRI imaging. FIG. 5B depicts a PET-CT image that demonstrates uptake of ⁸⁹Zr functionalized YAS microspheres following catheter-directed delivery.

FIGS. 6A and 6B provide images taken of a woodchuck. FIG. 6A provides an axial image from woodchuck with large single dominant hepatoma as shown in T2-weighted MRI image. FIG. 6B is a digital subtraction angiography of catheter located within the common hepatic artery just prior to delivery of ⁸⁹Zr functionalized YAS microspheres.

FIGS. 7A and 7B provide imaging following a scout dose (PT1; 7A) and full dose (PT2; 7B) catheter directed delivery of ⁸⁹Zr functionalized microspheres. Negligible lung uptake is noted, with appreciable differential uptake within tumor vs. normal liver.

### DETAILED DESCRIPTION

Some embodiments disclosed herein pertain to particles comprising therapeutic therapeutic, which may be used in therapeutic methods. In some embodiments, the particles are configured to be used in or are suitable for use in selective internal radiation therapy (SIRT). Some embodiments pertain to methods of making such radioisotopic particles and methods of using such radioisotopic particles for the treatment of cancer with improved dosimetry. In some embodiments, the particle is a nanoparticle or a microparticle (e.g., a microsphere). In some embodiments, a therapeutic radioisotope or precursor to a therapeutic radioisotope is functionalized (via chemical bonding) to the surface of a particle. In some embodiments, a therapeutic radioisotope or a precursor to a therapeutic radioisotope is functionalized to the surface of a particle comprising a therapeutic radioisotope or precursor to a therapeutic radioisotope to prepare the therapeutic radioisotopic particle or a precursor to the therapeutic radioisotopic particle. In some embodiments, the precursors radioisotopes may be activated by, for example, neutron bombardment to form the radioactive radioisotopes. In some embodiments, the functionalization is through a chemical bond. In some embodiments, the chemical bond is a Lewis acid-base interaction between the radioisotope and a surface of the substrate of the particle. In some embodiments, a therapeutic radioisotope or precursor to a therapeutic radioisotope is functionalized (via chemical bonding) to the surface of a particle to provide a therapeutic radioisotopic particle. In some embodiments, the therapeutic particle is mixed (or prepared with) separate therapeutic radioisotopic particles. In some embodiments, the mixture of particles can be used in methods as disclosed herein. Some embodiments relate to the field of therapy using therapeutic microspheres (or a population comprising a mix of therapeutic radioisotopic particles), including SIRT.

As used herein, the term “chemical bond” is given its plain and ordinary meaning and refers to a lasting attraction between atoms, ions or molecules that enables the formation of chemical compounds. The bond may result from the electrostatic force of attraction between oppositely charged ions as in ionic bonds or through the sharing of electrons as in covalent bonds. Chemical bonds include “strong” or “primary bonds” such as covalent, ionic and metallic bonds, and “weak bonds” or “secondary bonds” such as dipole-dipole interactions, the London dispersion force and hydrogen bonding.

As used herein, the term “coordinate bond” is given its plain and ordinary meaning and refers to a covalent bond in which both electrons come from the same atom.

As used herein, the term “covalent bond” is given its plain and ordinary meaning and refers to a bond between atoms formed by sharing a pair of electrons.

As used herein, the term “Lewis acid” is given its plain and ordinary meaning and refers to any species (molecule or ion) that is an electron-pair acceptor.

As used herein, the term “Lewis base” is given its plain and ordinary meaning and refers to any species (molecule or ion) that is an electron-pair donor.

As used herein, the term “Lewis acid-base adduct” is given its plain and ordinary meaning and refers to a compound that contains a coordinate covalent bond between a Lewis acid and a Lewis base.

As used herein, the term “half-life” or t_{1/2} is given its plain and ordinary meaning and refers to the time required for one-half of the atoms of a radioisotope to decay.

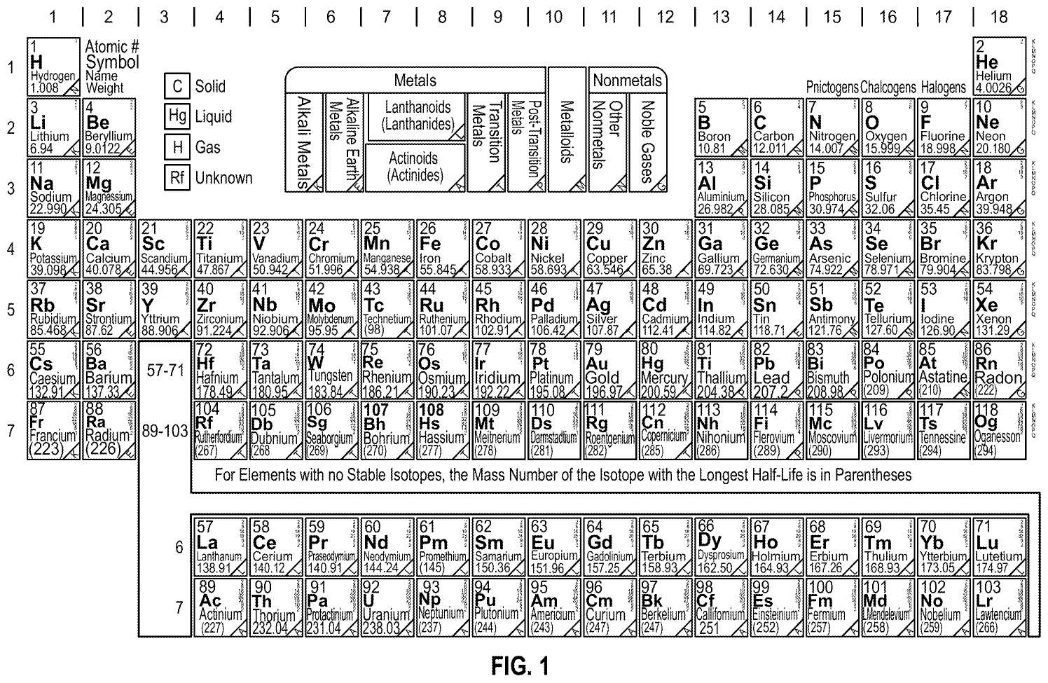

As used herein, the term “metalloid” refers to a type of chemical element which has properties in between, or that are a mixture of, those of metals and non-metals. Metalloids include, at least, boron (B), silicon (Si), germanium (Ge), arsenic (As), antimony (Sb), and tellurium (Te). The metalloids are shown in the periodic table of FIG. 1.

As used herein, the term “metal” refers to a type of chemical element of the periodic table that includes the alkali metals, the alkaline earth metals, and the transition metals. The transition metals further include the post-transition metals, lanthanides, and actinides. The metals are shown in the periodic table of FIG. 1.

As used herein, the term “non-metal” refers to a type of chemical element of the periodic table. The non-metals include carbon (C), nitrogen (N), oxygen (O), sulfur (S) and others as shown in the periodic table of FIG. 1.

As used herein the term “ceramic microsphere substrate” refers to a ceramic microsphere which forms the substrate to which the therapeutic radioisotope is bound.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, children, juveniles, adults and geriatric subjects. The subject can be a subject “in need of” the methods disclosed herein can be a subject that is experiencing a disease state, and the methods and compounds of the invention are used for assessing treatment options.

The term “effective amount,” as used herein, refers to that amount of a recited particle and/or composition that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, an effective amount can refer to the amount of a composition, particle, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Actual dosage levels of active ingredients in an active composition of the presently disclosed subject matter can be varied so as to administer an amount of the active particle(s) that is effective to achieve the desired response for a particular subject and/or application. The selected dosage level will depend upon a variety of factors including, but not limited to, the activity of the composition, route of administration, distribution of the composition, severity of the condition being treated, and the physical condition and prior medical history of the subject being treated. In some embodiments, a minimal dose is administered. Determination and adjustment of an effective dose, as well as evaluation of when and how to make such adjustments, are contemplated herein.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, curing the illness, etc.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, hydroxy, alkoxy, cyano, halogen, C-amido, N-amido, C-carboxy, O-carboxy, haloalkyl, haloalkoxy, a mercapto, an amino, a mono-substituted amino group, and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, or heteroaryl group. That is, the alkyl, alkenyl, alkynyl, ring of the cycloalkyl, ring of the cycloalkenyl, ring of the cycloalkynyl, ring of the aryl, or the ring of the heteroaryl can contain from “a” to “b”, inclusive, carbon atoms. Thus, for example, a “C₁ to C₄ alkyl” group refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁ to C₆ alkyl” group refers to all alkyl groups having from 1 to 6 carbons (e.g., 1, 2, 3, 4, 5, or 6). If no “a” and “b” are designated with regard to an alkyl, alkenyl, alkynyl, cycloalkyl cycloalkenyl, cycloalkynyl, aryl, or heteroaryl group, the broadest range described in these definitions is to be assumed.

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by, followed by the number of carbon atoms, followed by a “*”. For example, to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group (e.g., cyclohexyl).

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms, that is, an element other than carbon, including, but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s), or as otherwise noted herein. A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include, but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

As used herein, the term “amino” refers to a —NH₂ group.

As used herein, the term “hydroxy” refers to a —OH group.

As used herein, the term “cyano” refers to a “—CN” group.

As used herein, the term “mercapto” refers to an “—SH” group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, the term “C-amido” refers to a “—C(═O)N(R_{A}R_{B})” group in which R_{A} and R_{B} can be independently hydrogen, alkyl, alkenyl, alkynyl, cycloalkyl, cycloalkenyl, aryl, or heteroaryl. A C-amido may be substituted or unsubstituted.

An “N-amido” group refers to a “RC(═O)N(R_{A})—” group in which R and RA can be independently hydrogen, alkyl, alkenyl, alkynyl, cycloalkyl, cycloalkenyl, aryl, or heteroaryl. An N-amido may be substituted or unsubstituted.

As used herein, the term “O-carboxy” refers to a “RC(═O)O—” group in which R can be hydrogen, alkyl, alkenyl, alkynyl, cycloalkyl, cycloalkenyl, aryl, or heteroaryl, as defined herein. An O-carboxy may be substituted or unsubstituted.

As used herein, the terms “ester” and “C-carboxy” refer to a “—C(═O)OR” group in which R can be the same as defined with respect to O-carboxy. An ester and C-carboxy may be substituted or unsubstituted.

As used herein, the term “halogen atom” or “halogen” refers to any one of the radio-stable atoms of column 7 of the Periodic Table of the Elements, such as, fluorine, chlorine, bromine and iodine.

As used herein, “haloalkyl” refers to an alkyl group in which one or more of the hydrogen atoms are replaced by a halogen (e.g., mono-haloalkyl, di-haloalkyl and tri-haloalkyl). Such groups include, but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “diamino group” refers to a compound having two amino groups that are connected by an C₁₋₁₀alkyl chain, where the two amino groups independently optionally substituted (e.g., di-substituted amino groups or tri-substituted amino groups optionally substituted with additional C₁₋₆alkyl groups).

As used herein, “triamino group” refers to a compound having three amino groups that are connected by two or three C₁₋₁₀alkyl chains (e.g., forming a cyclic structure or a straight chain), where the three amino groups are independently optionally substituted (e.g., di-substituted amino groups or tri-substituted amino groups optionally substituted with additional C₁₋₆alkyl groups).

As disclosed elsewhere herein, in radioactive microspheres for use in SIRT can be delivered (e.g., via the trans catheter route) to a point in the vasculature from where they are carried by blood flow and/or injected fluid into the tissue of interest. Here they lodge in the capillaries and deliver a dose of therapeutic radiation, which is typically sufficient to cause localised tissue death. Therapeutic radiation typically is delivered in the form of beta or gamma radiation from beta or gamma emitting radioactive isotopes. A variety of therapeutic isotopes, including but not limited to yttrium-90 and holmium-166 may be used in SIRT. In one approach, glass microspheres comprising yttrium-90 (a beta emitter) are used for SIRT. These are prepared by neutron bombardment of non-radioactive glass microspheres comprising naturally occurring yttrium-89, which converts it to yttrium-90 by neutron capture.

The section headings used herein are for organizational purposes only and are not to be construed as limiting the described subject matter in any way. All literature and similar materials cited in this application including, but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

### INTRODUCTION

Beta radiation is the form of radiation used SIRT microspheres. Beta radiation which, while very desirable for tumor treatment, has a very short effective range. Prior to treatment, it is usual for the local vasculature to be mapped using an iodinated contrast agent, which allows the vessels to be visualised by X-ray based techniques. This allows the physician to better understand the local vascular anatomy and to plan delivery of the radioactive microspheres to the appropriate vessels feeding the tissue of interest.

In addition to mapping the vasculature, attempts to map the vasculature using microsphere surrogates before treatment have been made. This has been attempted using an imageable particulate surrogate, injected into the patient's vasculature to mimic the planned SIRT procedure. One imaging particulate surrogate used currently is a macro-aggregated albumen preparation, which is labelled with technetium-99m immediately before use (known as ⁹⁹mTcMAA). Technetium-99m is a short lived gamma emitter and may be imaged using a gamma-detecting camera. Single photon emission computed tomography (SPECT) imaging combines a gamma-detecting camera with x-ray imaging to yield images of the ⁹⁹mTcMAA emissions in context of the patient's anatomy. These images can then be used to elucidate the distribution of the ⁹⁹mTcMAA within the body and this information can be used to predict the final likely distribution of SIRT treatment microspheres. In this way, the proportion of delivered radiation dose that is predicted to be delivered to the tissue of interest can be calculated. Predictive dosimetry may be used to adjust the treatment plan to optimize the SIRT therapeutic dosing. The predicted distribution may also be used to triage patients suitable for treatment with SIRT. For example, if large amounts of microspheres are predicted to distribute to sensitive tissues such as lung or gut wall, it may be necessary to use angiographic techniques (e.g. coil embolization) to correct the flow of particles beyond the target tissue before proceeding with SIRT. If it is not possible to correct the non-target flow, then the SIRT dosing may need to be reduced to a safer level, or the patient may be considered unsuitable for treatment. Another approach was to prepare a resin or crystalline ceramic cores with imaging radioactive materials coated thereon. In still another application, microspheres may be prepared having an inactive radioactive isotope incorporated into a ceramic material. Their preparation typically uses activation of non-radioactive elements by neutron bombardment in a nuclear reactor.

Some embodiments disclosed herein provide new therapeutic radioisotopic particles (or a population comprising a mix of therapeutic radioisotopic particles and nontherapeutic radioisotopic particles) that allow SIRT using one or more of alpha emission, beta emission, positron emission, and/or gamma emission. In some embodiments, as disclosed in more detail elsewhere herein, therapeutic radioisotopes are chemically bonded to surfaces of particles to provide therapeutic radioisotopic particles. In some embodiments, a therapeutic particle is mixed (or prepared with) separate particles. In some embodiments, the mixture of particles can be used in methods as disclosed herein. For instance, in some embodiments, during the preparation of therapeutic particles therapeutic, blank particles are functionalized. Because only a portion of the blank (e.g., unfunctionalized) particles become functionalized, the resultant population may be a mixed population comprising blank particles and particles labeled with therapeutic radioisotopes.

### Therapeutic Radioisotopic Particles

As noted previously, some embodiments pertain to therapeutic radioisotopic particles (or a mixed population comprising therapeutic particles). In some embodiments, a therapeutic radioisotope is functionalized to the surface of a supporting substrate. This functionalization provides a therapeutic radioisotopic particle. In some embodiments, the supporting substrate is a particle. In some embodiments, the substrate provides the bulk of the particle (e.g., most of the size and/or weight of the particle may be attributed to the substrate). In some embodiments, the particle is a nanoparticle or microparticle. In some embodiments, the particle is a microsphere. In some embodiments, a therapeutic radioisotope is bound to the substrate (e.g., bound directly to the substrate) in a form where the therapeutic radioisotope is already therapeutic (e.g., the active therapeutic radioisotope). In other embodiments, a non-therapeutic radioisotope (e.g., a precursor to an therapeutic radioisotope) is bound to the substrate and is thereafter activated while on the substrate (e.g., by neutron bombardment).

In some embodiments, the substrate comprises a therapeutic radioisotope (or precursor thereto) during or prior to functionalization with another therapeutic radioisotope. In other embodiments, the substrate initially does not comprise a therapeutic radioisotope and the therapeutic radioisotope is functionalized to the particle (or activated by neutron bombardment).

In some embodiments, the therapeutic radioisotope is bound to the substrate via an irreversible linkage or substantially irreversible linkage. For instance, the therapeutic radioisotope is chemically bonded to the surface via one or more chemical bonds. In some embodiments, by providing a particle with a therapeutic radioisotope chemically bonded to the surface, risks associated with the therapeutic radioisotope detaching from the particle are lowered and/or extinguished. Moreover, because in some embodiments the therapeutic radioisotope can be functionalized to the substrate in their radioactive forms, there is no need for neutron activation of the therapeutic radioisotope. For example, in some embodiments, the therapeutic radioisotope is bound to the surface of the substrate during preparation of the therapeutic radioisotope via coupling to the substrate. In other embodiments, as mentioned elsewhere herein, however, activation of nonradioactive isotopes using conventional methods (e.g., neutron bombardment and capture) to form therapeutic radioisotopes while functionalized to the surface of a particle is envisioned.

As noted elsewhere herein, the therapeutic radioisotope may be functionalized to a surface of a substrate of a particle via a chemical bond. In some embodiments, the chemical bond is a primary bond. For example, in some embodiments, the therapeutic radioisotope is bonded to the surface of the particle (e.g., microsphere) through one or more covalent bonds. In some embodiments, the therapeutic radioisotope is bonded to the surface of the particle through one or more coordinate bonds. In some embodiments, the therapeutic radioisotope is bonded to the surface of the particle through a dative bond. In some embodiments, the therapeutic radioisotope is bonded to the surface of the particle through covalent bonds, coordinate bonds, dative bonds, ionic bonds, or combinations thereof. In some embodiments, the therapeutic radioisotope is bonded to the surface of the particle through a Lewis Acid-Base interaction (e.g., a Lewis acid-base coordination bond). For example, in some embodiments, one or more functional groups on the surface of a substrate of the particle act as Lewis bases and form a Lewis acid-base adduct with the therapeutic radioisotope (which act as a Lewis acids). In some embodiments, the Lewis base is an inorganic Lewis base.

As noted elsewhere herein, in some embodiments, the substrate of the particle provides a foundation to which the therapeutic radioisotope may be bound. In some embodiments, the substrate includes a core of the particle that extends outwardly (e.g., from the center of the particle) to the surface of the particle. In some embodiments, the substrate is an inorganic material. In some embodiments, while the substrate of the particle provides a foundation to which the therapeutic radioisotope may be bound, another therapeutic radioisotope may be provided within the substrate. For example, when functionalizing an yttrium aluminum silicon oxide substrate with a therapeutic radioisotope, the yttrium groups of the substrate may be therapeutic in nature.

As noted elsewhere herein, the substrate may be homogeneous or substantially homogeneous. To illustrate, the surface of the particle may comprise a number of atoms that are of overlapping elements and/or are the same elements (e.g., are atoms of the same elements) as found in the core of the substrate. In some embodiments, as disclosed elsewhere herein, a portion of the atoms that provide the surface of the substrate may be bound directly to the therapeutic radioisotope. The portion of atoms that are bound (e.g., chemically bonded) to the therapeutic radioisotope may be the same type of chemical element as atoms in the core of the particle. In some embodiments, the particle comprising the therapeutic radioisotope lacks any intervening molecular species or differing molecular species (such as linker groups) between the substrate and the therapeutic radioisotope. In some embodiments, the particle comprising the therapeutic radioisotope lacks any intervening organic molecular species (such as an organic linker groups) between the substrate and the therapeutic radioisotope.

In some embodiments, the substrate comprises an embedded therapeutic radioisotope. In some embodiments, the substrate comprises the embedded therapeutic radioisotope and a portion of the atoms that provide the surface of the substrate may be therapeutic radioisotopes. In some embodiments, the portion of atoms of the substrate that are bound (e.g., chemically bonded) to the therapeutic radioisotope may be the same chemical element as atoms in the core of the particle. In some embodiments, as disclosed elsewhere herein, the particle comprising the therapeutic radioisotope lacks any intervening molecular species or differing molecular species (such as linker groups) between the substrate and the therapeutic radioisotope.

In some embodiments, as disclosed elsewhere herein, the substrate comprises an inorganic material. In some embodiments, the inorganic material comprises one or more elements that are metalloids, metals, or both (as defined on the periodic table). In some embodiments, one or more of the metals present are embedded therapeutic radioisotopes (or precursors thereto, which may be activated by neutron bombardment, etc.) In some embodiments, the inorganic material further comprises an element that is a non-metal. In some embodiments, the inorganic material comprises at least one non-metal, metalloid, metal, or combinations thereof. In some embodiments, the inorganic material comprises at least one metalloid oxide, metal oxide, or combinations thereof. In some embodiments, the inorganic material comprises at least one transition metal oxide. In some embodiments, the metalloid atoms, metal atoms, or both are bonded to non-metal atoms, forming the substrate structure. For example, in a substrate comprising a transition metal oxide, the oxygen of the oxide is considered the non-metal chemical element providing at least a portion of the substrate.

In some embodiments, the substrate comprises a crystal lattice, an amorphous structure, or combinations thereof. In some embodiments, the core of the substrate comprises a first portion of metalloid or metal atoms bonded to the non-metal atoms while the surface comprising a second portion of the metalloid or metal atoms bonded to the non-metal atoms. In some embodiments, as disclosed elsewhere herein, the therapeutic radioisotope may be bound directly to the substrate through at least a portion of the non-metal atoms at the surface of the substrate.

As will be readily apparent, the substrate of the particles may be made of a variety of materials, such as one or more inorganic materials. In some embodiments, the substrate is an inorganic material. In some embodiments, the inorganic material comprises a ceramic material and/or is ceramic. In some embodiments, the inorganic material comprises at least one element selected from silicon, yttrium, manganese, aluminium, gallium, strontium, and titanium. In some embodiments, the inorganic material comprises Pb, Al, Si, Y, Mn, Ga, Fe, Ti, Lu, Y, I, Sr, Sm, Ra, At, Ac, Th, Bi, or combinations thereof. In some embodiments, the inorganic material comprises glass or is glass. In some embodiments, the inorganic material comprises silicon dioxide. In some embodiments, the inorganic material comprises silicon dioxide and at least one other element selected from yttrium, manganese, aluminium, gallium, boron, strontium, and titanium. In some embodiments, the inorganic material comprises one or more of SiO₂, MnO₂, AlO₃, Ga₂O₃, Fe₂O₃, SrO₂, and/or TiO₂. In some embodiments, the inorganic material comprises SiO₂ and one or more of MnO₂, AlO₃, Ga₂O₃, Fe₂O₃, and/or TiO₂. In some embodiments, the inorganic material comprises SiO₂ and one or more of Al₂O₃ and/or Y₂O₃.

In some embodiments, the substrate comprises a ceramic material and/or is ceramic. In some embodiments, the substrate comprises at least one element selected from silicon, yttrium, manganese, aluminium, gallium, strontium, and titanium. In some embodiments, the inorganic material comprises glass or is glass. In some embodiments, the substrate comprises silicon dioxide. In some embodiments, the substrate comprises silicon dioxide and at least one other element selected from yttrium, manganese, aluminium, gallium, boron, strontium, and titanium. In some embodiments, the substrate comprises one or more of SiO₂, MnO₂, AlO₃, Ga₂O₃, Fe₂O₃, SrO₂, and/or TiO₂. In some embodiments, the substrate comprises SiO₂ and one or more of MnO₂, AlO₃, Ga₂O₃, Fe₂O₃, SrO₂, and/or TiO₂. In some embodiments, the substrate comprises SiO₂ and one or more of Al₂O₃ and/or Y₂O₃.

In some embodiments, the substrate comprises yttrium aluminum silicon oxide. In some embodiments, the yttrium aluminum silicon oxide is composed of 17Y₂O₃-19Al₂O₃-64SiO₂ by mol %. In some embodiments, the substrate of the therapeutic particles disclosed herein are TheraSphere® (Biocompatibles, UK, Ltd.). TheraSphere consists of insoluble glass microspheres where yttrium is an integral constituent of the glass (i.e., the TheraSphere® comprises an yttrium aluminum silicon oxide). The yttrium in the precursor particles in TheraSphere® is in the form of the naturally occurring non-radioactive isotope ⁸⁹Y. ⁸⁹Y is not a beta emitter and is not radiotherapeutic. In some embodiments, the substrate used for the therapeutic radioisotopic particles disclosed herein are TheraSphere particles including ⁸⁹Y and lacking ⁹⁰Y. In some embodiments, the yttrium in yttrium aluminum silicon oxide substrate as disclosed herein is provided in its abundant natural form (⁸⁹Y), which does not emit beta radiation.

In some embodiments, a therapeutic radioisotope precursor is functionalized to the surface of the substrate in its nonradioactive, non-therapeutic form (e.g., as a therapeutic radioisotope precursor) and is activated while on the substrate. In some embodiments, the therapeutic isotope precursor is attached to the particle and is activated thereafter to provide the therapeutic radioisotopic particle. In some embodiments, the activation is through neutron bombardment and capture. In some embodiments, the therapeutic radioisotope precursor is selected from one or more of Th, Cr, Ga, In, Cu, Zr, Fe, K, Rb, Na, Ti, Sc, Cr, AlF, and Lu. In some embodiments, the therapeutic radioisotope (after activation or as functionalized to the substrate) is selected from one or more of ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, Al¹⁸F, and ¹⁷⁷Lu. In some embodiments, the therapeutic radioisotope precursor is selected from one or more of Lu, Y, I, Sr, Sm, Ra, At, Ac, Th, Bi, and/or Pb. In some embodiments, the therapeutic radioisotope (after activation or as functionalized to the substrate) is selected from one or more of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

In some embodiments, as disclosed elsewhere herein, the substrate comprises a single material, while in others, it can comprise more than one material. Where the substrate is a substantially homogeneous material, in some embodiments, it may comprise a substantially homogeneous mixture of constituent elements (e.g., Si and O in SiO₂). Where the substrate is homogeneous, the surface also comprises at least a portion of those constituent elements (e.g., Si and O atoms), though the surface may also comprise a terminating atom (such as a —H in an —OH). In some embodiments, atoms that are provided within the core are also provided on the surface as part of terminal functional groups (e.g., an O in a —OH). As noted above, the terminal functional groups may additionally comprise terminal atoms (e.g., the —H of an —OH). Such an arrangement is provided below for illustration.

The particle, where made from SiO₂, may be represented by the following structure (I):

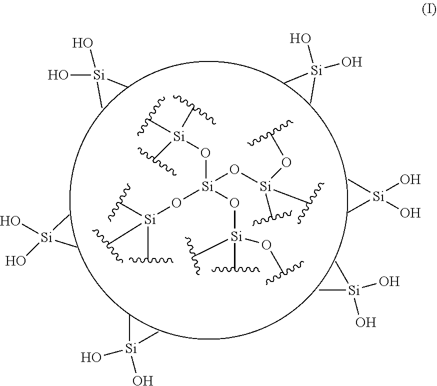

While the structure of Formula (I) comprises terminal groups that may be substantially absent in the core of the substrate, this particle would still be considered homogeneous because the elements at the surface that are not constituents of the core are provided as terminal groups.

More generally, in some embodiments, the particle may be represented as the structure having Formula (II):

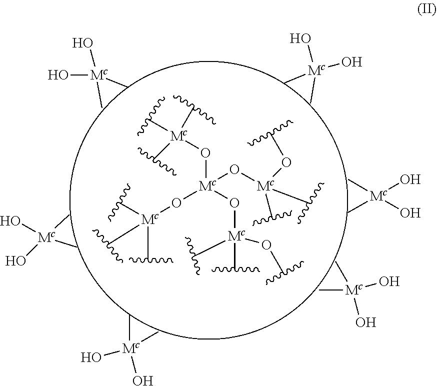

where each instance of M^{c} is independently selected from the group consisting of Pb, Al, Si, Y, Mn, Ga, Fe, Ti, Lu, Y, I, Sr, Sm, Ra, At, Ac, Th, and/or Bi. In some embodiments, M^{c} is independently selected from the group consisting of Pb, Al, Si, Y, Mn, Ga, Fe, Sr, and Ti. In some embodiments, as disclosed elsewhere herein, the substrate comprises a therapeutic isotope and/or a precursor to a therapeutic radioisotope (e.g., a nonradioactive isotope that can be activated by neutron bombardment and capture, etc.). In some embodiments, each instance of M^{c} is independently selected from the group consisting of Pb, Al, Si, Y, Mn, Ga, Fe, Ti, Lu, Y, I, Sr, Sm, Ra, At, Ac, Th, and/or Bi. In some embodiments, Lu, Y, I, Sr, Sm, Ra, At, Ac, Th, Bi, and/or Pb are provided as precursors to radioactive therapeutic isotopes and may be activated to provide a therapeutic radioisotope selected from ¹⁷⁷Lu, ⁹⁰Y, ¹³¹I, ⁸⁹Sr, ¹⁵³m, ¹⁴⁹Tb, ²²³Ra, ²²⁴Ra, ²¹¹At, ²²⁵Ac, ²²⁷Th, ²¹²Bi, ²¹³Bi, and/or ²¹²Pb (or combinations thereof). In some embodiments, these isotopes are activated before or after the addition of a therapeutic radioisotope and/or a precursor to a therapeutic radioisotope. In some embodiments, M^{c} each instance of M^{c} is independently selected from Si, Y, and Al (e.g., an yttrium aluminum silicon oxide). In some embodiments, when the surface of the substrate is functionalized, the one or more OH groups at the surface can comprise M^{b}(X)ₙ and/or M^{d}(X)ₙ as disclosed elsewhere herein. In some embodiments, where the substrate comprises precursors to radioactive therapeutic isotopes, the precursors to radioactive therapeutic isotopes may be activated before or after functionalization of the surface with M^{b}(X)ₙ(and/or M^{d}(X)ₙ).

In some embodiments, the terminal groups of the substrate provide functional groups may interact with a therapeutic radioisotope and/or a precursor of a therapeutic radioisotope to chemically bond to the therapeutic radioisotope and/or precursor of a therapeutic radioisotope, thereby forming the therapeutic particle and/or a precursor to the therapeutic particle. In some embodiments, the precursor is a nonradioactive isotope that can be activated by neutron bombardment and capture, etc. to provide the radioactive radioisotope. In some embodiments, the therapeutic radioisotope and/or a precursor to the therapeutic radioisotope is chemically bonded directly to the surface of the substrate of the particle. In some embodiments, as exemplified in the structures elsewhere herein, an inorganic substrate may comprise a surface having one or more electron donating functionalities (e.g., —OH) that coordinate or covalently bond to a therapeutic radioisotope, a bridging atom, the therapeutic radioisotope, or both, thereby bonding it to the surface.

As noted above, the therapeutic radioisotope, the precursor to the therapeutic radioisotope, or combinations thereof may decorate the surface of the particle to provide a therapeutic particle or the therapeutic particle. In some embodiments, on average, each particle (e.g., microsphere) comprises a plurality of therapeutic radioisotopes (or precursors thereof) bound to the substrate (e.g., 2, 3, or 4). In some embodiments, not every microsphere is functionalized with a therapeutic radioisotope. In some embodiments, the number of therapeutic radioisotopes per particle (e.g., functionalized to each particle) is equal to or less than about: 1×10⁻⁶, 0.001, 0.01, 0.1, 0.5, 1, 2, or ranges including and/or spanning the aforementioned values. In some embodiments, a particle lacks a therapeutic radioisotope on its surface (and/or that otherwise lacks a therapeutic radioisotope) and that lacks a therapeutic radioisotope on its surface is a blank particle.

In some embodiments, the therapeutic radioisotope may be a metal. In some embodiments, the therapeutic feature of the particle may be a host metal bonded to a therapeutic radioactive isotope that is not a metal. For instance, a complex with a non-metal radioisotope such as Al¹⁸F. In some embodiments, this complex chemically bonds to the surface via the aluminium atom with ¹⁸F being complexed to the aluminium atom. In some embodiments, the therapeutic radioisotope is selected from technetium-99m (⁹⁹mTc), thallium-201 (²⁰¹Th), chromium-51 (⁵¹Cr), gallium-67 (⁶⁷Ga), gallium-68 (⁶⁸Ga), indium-111 (¹¹¹In), copper-64 (⁶⁴Cu), zirconium-89 (⁸⁹Zr), iron-59 (⁵⁹Fe), potassium-42 (⁴²K), rubidium-82 (⁸²Rb), sodium-24 (²⁴Na), titanium-45 (⁴⁵Ti), scandium-44 (44Sc), chromium-51 (⁵¹Cr), fluorine-18 (¹⁸F), lutetium-177 (¹⁷⁷Lu), and/or combinations thereof. In some embodiments, the precursor to the therapeutic radioisotope is selected from technetium, thallium, chromium, gallium, indium, copper, zirconium, iron, potassium, rubidium, sodium, titanium, scandium, chromium, fluorine, lutetium, and/or combinations thereof.

In some embodiments, the therapeutic radioisotope may be a metal. In some embodiments, the therapeutic radioisotope is selected from lutetium-177 (¹⁷⁷Lu), yttrium-90 (⁹⁰Y), iodine-131 (¹³¹I), strontium-89 (⁸⁹Sr), samarium-153 (¹⁵³Sm), radon-223 (²²³Ra), radon-224 (²²⁴Ra), astatine-211 (²¹¹At), actinium-225 (²²⁵Ac), thorium-227 (²²⁷Th), bismuth-212 (²¹²Bi), bismuth-213 (²¹³Bi), lead-212 (²¹²Pb), and/or combinations thereof. In some embodiments, the precursor of the therapeutic radioisotope is selected from lutetium, yttrium, iodine, strontium, samarium, radon, astatine, actinium, thorium, bismuth, lead, and/or combinations thereof. In some embodiments, the therapeutic radioisotope is an alpha emitter or a beta emitter.

As noted above, in some embodiments, the therapeutic radioisotope, the precursor to the therapeutic radioisotope, and combinations thereof is chemically bonded to the surface of the substrate via one or more chemical bonds. In some embodiments, the therapeutic radioisotope may also be (or alternatively be) functionalized to a surface of the substrate of a particle at least in part through an inorganic bridge. An inorganic bridge is a series of atoms bonded together that lacks an organic portion. As used herein, the term inorganic is used in its conventional sense and would be understood one of skill in the art to refer to compounds (or portions thereof or atoms thereof) that lack organic carbon-based portions (such as alkyls, etc.). Inorganic, as used herein, does not include organometallic entities.

Where present, the inorganic bridge comprises a metal atom or metalloid atom that is not the radioisotope (e.g., a bridging metal atom). The inorganic bridge also comprises one or more non-metal atoms chemically bonded with the bridging atom (e.g., the bridging metal atom) in a series that spans between the therapeutic radioisotope and the substrate, in other words, bridging between the therapeutic radioisotope and the substrate. In some embodiments, each atom making the inorganic bridge is chemically bonded to another atom in the inorganic bridge thereby connecting the substrate and the therapeutic radioisotope through chemical bonds. In some embodiments, the inorganic bridge comprises a non-metal atom of the substrate, a bridging atom (e.g., a bridging metal atom), and a non-metal atom that is, in turn, chemically bonded to the therapeutic radioisotope. In some embodiments, the non-metal atom of the substrate is chemically bonded to the bridging atom (e.g., a bridging metal atom) and the bridging atom is chemically bonded to the non-metal atom that is chemically bonded to the therapeutic radioisotope.

In some embodiments, the bridging metal atom (e.g., Sn) initially acts as a reducing agent for the therapeutic radioisotope (e.g., ^{99m}Tc) during functionalization of the substrate with the therapeutic radioisotope. The bridging atom then may remain chemically bonded (e.g., through chemical bonding such as covalent bonding, coordinate bonding, and/or Lewis acid base interactions) between the therapeutic radioisotope and the substrate. The therapeutic radioisotope and the bridging metal atom may be separated by a non-metal atom (e.g., O) that may be chemically bonded to both the therapeutic radioisotope and the bridging metal atom. This non-metal atom may be part of the inorganic bridge. Similarly, a non-metal atom of the substrate (e.g., O) may be directly bonded to the bridging metal atom connecting the bridging metal atom to the substrate. Without being bound by any particular mechanism, it is believed that tin (Sn) is a bridging atom for ^{99m}Tc and acts as part of an inorganic bridge between ^{99m}Tc and the substrate. In some embodiments, the inorganic bridge comprises or consists of —O—Sn—O— that chemically connects (e.g., through chemical bonding) the substrate to the therapeutic radioisotope (e.g., ^{99m}Tc). In some embodiments, the inorganic bridge comprises an —O—Sn—O— bridge may be further bonded to one or more of —OH, ═O, and —O⁻ (e.g., as —O—Sn(X)ₙ—O—, where each instance of X is —OH, ═O, and —O⁻ and n is 1 or 2).

In some embodiments, the therapeutic radioisotope may bind both directly to the substrate through atoms of the substrate (e.g., non-metal atoms such as O) and via an inorganic bridge (e.g., through the metal atom that is not the radioisotope) simultaneously. Such a configuration is shown in certain configurations of Formula (VIII) below. In other embodiments, the metal atom that is not the radioisotope forms chemically bonded bridge between the radioisotope and the surface of the substrate and the radioisotope itself is chemically bonded to the substrate only through the bridging metal atom (or multiple bridging metal atoms), as shown in some configurations of Formula (V) below. For example, as shown in Formula (V), the bridging metal can be chemically bonded to two non-metals where the two non-metal atoms (e.g., O) are further both chemically bonded to the therapeutic radioisotope.

In some embodiments, the therapeutic radioisotope may be a non-metal. In some embodiments, the therapeutic particle may comprise and feature that may be a metal bonded to a therapeutic radioactive isotope that is not a metal. For instance, a complex between a metal and a therapeutic non-metal radioisotope such as Al¹⁸F. In some embodiments, this complex chemically bonds to the surface via the aluminium atom with ¹⁸F being complexed to the aluminium atom. In some embodiments, the complex is Al¹⁸F.

As noted above, in some embodiments, the therapeutic radioisotope is coupled to the surface of a particle via the substrate of the particle. In some embodiments, the therapeutic particle may also (e.g., along with one or more of Formulae (I) to (II)) or alternatively be represented by Formula (III):

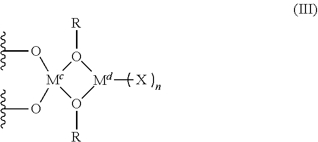

where the substrate comprises M^{c} and M^{b} is a therapeutic radioisotope. In some embodiments, M^{c} is selected from Pb, Al, Si, Y, Mn, Ga, Fe, Ti, Lu, Y, I, Sr, Sm, Ra, At, Ac, Th, and/or Bi; M^{b} is selected from ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, Al¹⁸F, and ¹⁷⁷Lu; each instance of R is not present or is —H; each instance of X is independently selected from —OH, ═O, and —O⁻; and n is an integer selected from 0, 1, 2, 3, or 4. In some embodiments, where a therapeutic precursor is provided in the substrate, it may be activated to provide ¹⁷⁷Lu, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb. In some embodiments, M^{c} is selected from Sn, Si, Mn, Al, Ga, Fe, Ti, and Pb; M^{b} is selected from ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, Al¹⁸F, and ¹⁷⁷Lu; each instance of R is not present or is —H; each instance of X is independently selected from —OH, ═O, and —O—; and n is an integer selected from 0, 1, 2, 3, or 4.

In some embodiments, the particle may also (e.g., along with one or more of Formulae (I) to (III)) or alternatively be represented by Formula (IV):

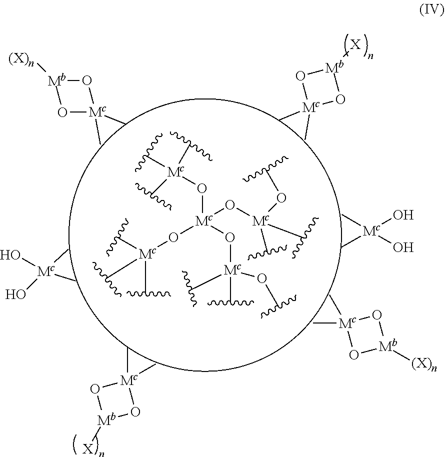

where the variables are as defined elsewhere herein. In some embodiments, as disclosed elsewhere herein, the substrate comprises M^{c} and M^{b} is a therapeutic radioisotope. In some embodiments, each instance of M^{c} is independently selected from Pb, Al, Si, Y, Mn, Ga, Fe, Ti, Lu, Y, I, Sr, Sm, Ra, At, Ac, Th, and/or Bi; each instance of M^{b} is independently selected from ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, Al¹⁸F, ¹⁷⁷Lu; each instance of R is either not present or is —H; each instance of X is independently selected from —OH, ═O, and —O—; and n is an integer selected from 0, 1, 2, 3, or 4. In some embodiments, each instance of M^{c} is selected from Si, Al, and Y; M^{b} is ⁸⁹Zr; each instance of X is selected from —OH and Sn; and n is 2 or 3. In some embodiments, each instance of M^{c} is selected from Si, Al, and Y; M^{b} is ⁸⁹Zr; each instance of X is-OH; and n is 2. In some embodiments, M^{c} is Si; M^{b} is and ⁸⁹Zr; X is —OH; and n is 2. In some embodiments, where a therapeutic precursor is provided in the substrate, it may be activated to provided ¹⁷⁷Lu, ¹³¹I, ⁸⁹S, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

In some embodiments, the particle may also (e.g., along with one or more of Formulae (I) to (IV)) or alternatively be represented by Formula (V):

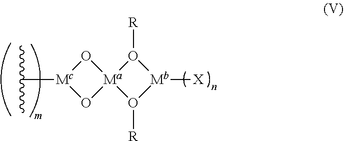

M^{a} may either be an atom of the substrate or a bridging metal atom that connects the therapeutic radioisotope to the substrate through chemical bonds. In some embodiments, for example, M^{a} is either an atom of the substrate or a bridging atom and M^{a} is selected from Pb, Al, Si, Y, Mn, Ga, Fe, Ti, Lu, Y, I, Sr, Sm, Ra, At, Ac, Th, Bi, and Sn; the substrate comprises M^{c} and M^{c} is independently selected from Pb, Al, Si, Y, Mn, Ga, Fe, Ti, Lu, Y, I, Sr, Sm, Ra, At, Ac, Th, and/or Bi; m is an integer selected from 1, 2, or 3; M^{b} is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁷⁷Lu, Al¹⁸F, and/or combinations thereof; each instance of R is independently either not present or is —H; each instance of X is independently selected from —OH, ═O, —O⁻, a mono-substituted amino group, a di-substituted amino group, halogen, —CN, —CF₃, an optionally substituted diamino group, an optionally substituted triamino group, wherein the substituent or substituents of the amino group, where present, are independently C₁₋₆ alkyl, heteroaryl, or aryl; n is an integer selected from 0, 1, 2, 3, or 4; and m is an integer equal to 1, 2, or 3. In some embodiments, each instance of R is either not present or is H; X is selected from —OH, ═O, and —O⁻; and n is an integer selected from 0, 1, 2, 3, or 4. In some embodiments, M^{a} is Sn and M^{a} is a bridging metal atom. In some embodiments, the substrate comprises M^{a} and M^{c} while M^{b} is a therapeutic radioisotope. In some embodiments, M^{a} and M^{c} are independently selected from Pb, Al, Si, Y, Mn, Ga, Fe, Ti, Lu, Y, I, Sr, Sm, Ra, At, Ac, Th, and/or Bi; M^{b} is selected from ²⁰¹Th, ⁵¹Cr, ⁶⁷Ga, ⁶⁸Ga, ¹¹¹In ⁶⁴Cu, ⁸⁹Zr, ⁵⁹Fe, ⁴²K, ⁸²Rb, ²⁴Na, ⁴⁵Ti, ⁴⁴Sc, ⁵¹Cr, Al¹⁸F, and ¹⁷⁷Lu; each instance of R is independently either not present or is —H; each instance of X is independently selected from ═O, —O⁻, —OH, a mono-substituted amino group, a di-substituted amino group, halogen, —CN, —CF₃, an optionally substituted diamino group, an optionally substituted triamino group, wherein the substituent or substituents of the amino group, where present, are independently C₁₋₆ alkyl, heteroaryl, or aryl; n is an integer selected from 0, 1, 2, 3, or 4; and m is an integer equal to 1, 2, or 3. In some embodiments, M^{a} is Si, Y, and Al; M^{b} is ⁸⁹Zr, M^{c} is selected from Si, Al, and Y; X is —OH; and n is 2. In some embodiments, M^{c} is Si, Al, or Y; M^{a} is Sn; M^{b} is ⁹⁹mTc; each X is independently —OH or ═O; and n is 2 or 3. In some embodiments, M^{b} is ⁹⁹mTc, X is —OH, and n is 2 or 3. Alternatively, in some embodiments, M^{b} may be a therapeutic radioisotope comprising a host metal bonded to a radioactive isotope that is not a metal. For instance, the complex with a non-metal radioisotope such as Al¹⁸F. In some embodiments, this complex chemically bonds to the surface via the aluminium atom with ¹⁸F being complexed to the aluminium atom. In some embodiments, M^{a} and M^{c} are independently selected from Si, Al, Y, and Sn; M^{b} is selected from ⁹⁹mTc and ⁸⁹Zr; each instance of X is —OH; and n is 2 or 3. In some embodiments, M^{a} and M^{c} are independently selected from Si, Al, Y, Sn; M^{b} is ⁹⁹mTc; each instance of X is —OH; and n is 2 or 3. In some embodiments, M^{a} and M^{c} are independently selected from Si, Al, Y, and Sn; M^{b} is ⁹⁹mTc; each instance of X is —OH; and n is 3. In some embodiments, M^{a} and M^{c} are independently from Si, Al, and Y; M^{b} is ⁸⁹Zr; each instance of X is-OH; and n is 2. In some embodiments, M^{a} is Si; M^{c} is Al; M^{b} is ⁸⁹Zr; X is —OH; and n is 2. In some embodiments, M^{a} is Si; M^{c} is Al; M^{b} is ⁹⁹mTc; each instance of X is independently selected from —OH and Sn; and n is 3. In some embodiments, R is —H. In some embodiments, where a therapeutic precursor is provided in the substrate, it may be activated (e.g., by neutron capture) to provide ¹⁷⁷Lu, ¹³¹I, ⁸⁹Sr, ¹⁵³m, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

In some embodiments, the particle may also (e.g., along with one or more of Formulae (I) to (V)) or alternatively be represented by Formula (VI):

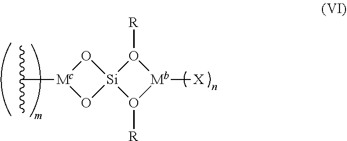

where M^{c}, M^{b}, R, X, and n are as disclosed elsewhere herein.

In some embodiments, the particle may also (e.g., along with one or more of Formulae (I) to (VI)) or alternatively be represented by Formula (VII):

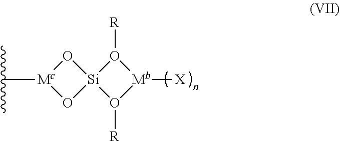

where M^{c}, M^{b}, R, X, and n are as disclosed elsewhere herein.

In some embodiments, the particle may also (e.g., along with one or more of Formulae (I) to (VII)) or alternatively be represented by Formula (VIII):

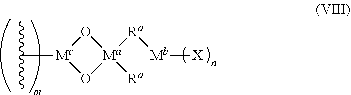

where M^{c}, M^{b}, X, and n are as disclosed elsewhere herein. In some embodiments, the substrate comprises M^{a} and M^{c} and M^{a} and M^{c} are independently selected from Pb, Al, Si, Y, Mn, Ga, Fe, and Ti; m is an integer selected from 1, 2, or 3; M^{b} is selected from ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁷⁷Lu, Al¹⁸F, and/or combinations thereof; M^{a} is either an atom of the substrate or a bridging atom and M^{a} is selected from Sn, Pb, Al, Si, Y, Mn, Ga, Fe, and Ti; each instance of R^{a} is independently OH, O, —O—Sn(X)ₙ—O or —OSnO—; each instance of X is selected from —OH, ═O, and —O—; and each instance of n is an integer selected from 0, 1, 2, 3, or 4. In some embodiments, M^{c} is Al; M^{a} is Si; M^{b} is ^{99m}Tc; each X is independently —OH or ═O; and n is 2 or 3. In some embodiments, M^{c} is Al; M^{a} is Si; M^{b} is ^{99m}Tc; at least an instance of R^{a} is —O—Sn—O—, each X is independently —OH or ═O; and n is 2 or 3. In some embodiments, M^{c} is Al; M^{a} is Si; M^{b} is ^{99m}Tc; at least an instance of R^{a} is —O—Sn(X)ₙ—O—, each X is independently —OH or ═O; and each instance of n is independently 2 or 3. In some embodiments, M^{b} is ^{99m}Tc; at least an instance of R^{a} is —O—Sn—O—, each X is independently —OH or ═O; and n is 2 or 3. In some embodiments, M^{b} is ^{99m}Tc; at least an instance of R^{a} is —O—Sn(X)ₙ—O—, each X is-OH; and n is 2. In some embodiments, M^{b} is ^{99m}Tc; an instance of R^{a} is —O—Sn—O—, an instance of R^{a} is —O— or —OH—; each X is independently —OH or ═O; and n is 2 or 3. In some embodiments, M^{b} is ^{99m}Tc; an instance of R^{a} is —O—Sn(X)ₙ—O—, an instance of R^{a} is —O— or —OH—; each X is independently —OH or ═O; and n is 2 or 3.

In some embodiments, the particle may also (e.g., along with one or more of Formulae (I) to (VIII)) or alternatively be represented by Formula (IX):

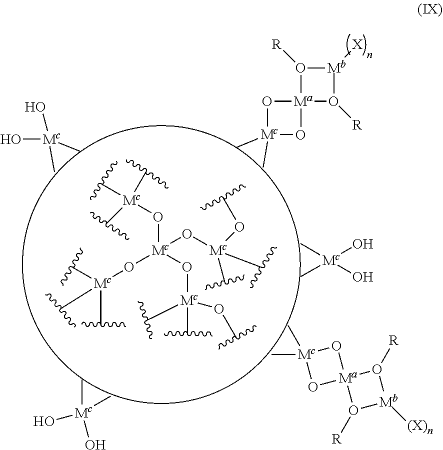

where the variables are as defined elsewhere herein.

In some embodiments, the particle may also (e.g., along with one or more of Formulae (I) to (IX)) or alternatively be represented by Formula (X):

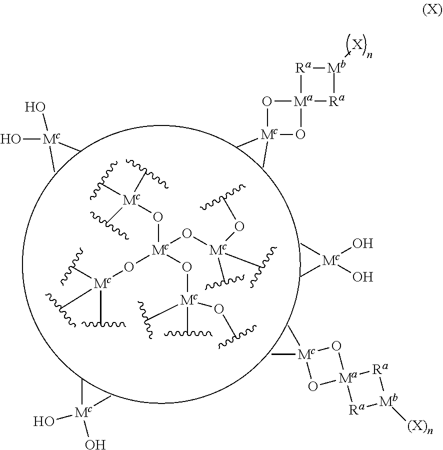

where the variables are as defined elsewhere herein.

In some embodiments, in addition to the structure of any one or more of Formulae (I) to (X), the particle may also include Formula (XI):

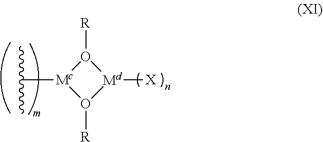

where the substrate comprises M^{c} and M^{d} is therapeutic radioisotope or a precursor to a therapeutic radioisotope. In some embodiments, M^{c} is as disclosed anywhere else herein. In some embodiments, M^{d} is selected from ¹⁷⁷Lu, ⁹⁰Y¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb. In some embodiments, M^{d} is a precursor of any one of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb (e.g., Lu, Y, I, Sr, Sm, Ra, At, Ac, Th, and/or Bi). In some embodiments, M^{c} is selected from Pb, Al, Si, Y, Mn, Ga, Fe, Ti, Lu, Y, I, Sr, Sm, Ra, At, Ac, Th, and/or Bi; M^{d} is selected from ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb; each instance of R is independently either not present or is —H; each instance of X is independently selected from ═O, —O⁻, —OH, a mono-substituted amino group, a di-substituted amino group, halogen, —CN, —CF₃, an optionally substituted diamino group, an optionally substituted triamino group, wherein the substituent or substituents of the amino group, where present, are independently C₁₋₆ alkyl, heteroaryl, or aryl; n is an integer selected from 0, 1, 2, 3, or 4; and m is an integer equal to 1, 2, or 3. In some embodiments, M^{c} is selected from the group consisting of Si and Al, X is —OH, and n is 2. In some embodiments, X is —OH; and n is 2 or 3. In some embodiments, M^{c} is selected from Si, Al, and Y; each instance of X is selected from —O⁻ and —OH; and n is 2 or 3. In some embodiments, R is —H. In some embodiments, X is —OH.

In some embodiments, the therapeutic radioisotope may be selected based on its half-life. For instance, in some embodiments, therapeutic radioisotopes with shorter half-lives are selected so that the body is exposed to radiation from the therapeutic radioisotope for shorter periods of time. In some embodiments, the therapeutic radioisotope has a half-life of less than or equal to about: 1 day, 3 days, 7 days, 2 weeks, one month, two months, or ranges including and/or spanning the aforementioned values.

In some embodiments, as disclosed elsewhere herein, the therapeutic element (e.g., radioisotope) is confined to the surface of the microsphere. In some embodiments, the substrate lacks a therapeutic radioisotope.

As noted elsewhere herein, in some embodiments, the substrate is porous. In other embodiments, the substrate is non-porous. In some embodiments, porosity of the particles herein is measured by their surface area per unit weight. In some embodiments, the surface area of the substrate of the particles as disclosed herein is less than or equal to about: 1 m²/g, 0.5 m²/g, 0.25 m²/g, 0.1 m²/g, 0.05 m²/g, or ranges including and/or spanning the aforementioned values. In some embodiments, where the particle is substantially non-porous or lacks porous, the radioisotope may be bound to a peripheral surface of the particle (e.g., a surface that is not within a pore and that is not internal to the outer circumference of the particle). In other embodiments, where the particle is porous, the radioisotope may be bound to any surface of the particle, including within a cavity or pore of the particle and/or to a peripheral surface of the particle.

In some embodiments, the particles are microspheres. A microsphere is a particle having microscale dimensions. In some embodiments, the microspheres have an average size of between 5 μm and 1000 μm. In some embodiments, the particles are microscale structures with an average size between 20 μm and 30 μm or between 15 μm and 100 μm. In some embodiments, the average size of the microparticles is less than or equal to about: 500 nm, 1000 nm, 5 μm, 20 μm, 25 μm, 30 μm, 40 μm, 50 μm, 100 μm, 250 μm, 500 μm, 750 μm, 1000 μm, or ranges including and/or spanning the aforementioned values. In some embodiments, the particles are nanoparticles. A nanoparticle is a particle having nanoscale dimensions. In some embodiments, the average size of the therapeutic particles is less than or equal to about: 100 nm, 250 nm, 500 nm, 750 nm, 1000 nm, or ranges including and/or spanning the aforementioned values.

The diameter of the particles can be easily determined by light or electron microscopy.

In some embodiments, the substrate for the therapeutic particles (or the population comprising therapeutic particles) is selected for its similarity for example in size, shape, density and/or chemical composition, to particles currently used for SIRT. For example, TheraSphere® consists of insoluble glass microspheres where yttrium-90 is an integral constituent of the glass. These radioactive glass microspheres are about 20 μm to 30 μm in diameter. In some embodiments, each milligram of therapeutic radioisotopic particles (or the population comprising therapeutic particles) comprises equal to or less than about: 5,000, 10,000 particles, 20,000 particles, 30,000 particles, 50,000 particles, 70,000 particles, 80,000 particles, 100,000 particles, or ranges including and/or spanning the aforementioned values.

While in several embodiments disclosed herein, reference is made to the use of a microsphere, the term particle broadly includes microspheres and other particles onto which the or therapeutic radioisotope may be bound. For example, the particle can vary in size and shape (e.g., cylindrical, cubic, pyramidal, box-shaped, etc.). In some embodiments, the shape of the particle is selected depending on the corresponding size and shape of the surrogate for the therapeutic particle.

In some embodiments, the therapeutic microsphere is configured to not be exposed to neutron bombardment and/or neutron activation to transform a non-therapeutic isotope of an element into the therapeutic radioisotope of the therapeutic microsphere. In some embodiments, the therapeutic agent is not a paramagnetic material and/or is not a therapeutic agent selected from the group consisting of H-1, He-3, Li-7, B-7, B-9, N-15, 0-17, F-19, Mg-27, Al-27, Si-29, S-33, C₁₋₃₇, Ca-43, Ti-47, V-51, Cr-53, Mn-55, Fe-57, Ni-61, Cu-63, Zn-67, Ga-69, Ge-73, Kr-83, Sr-87, Y-89, Zr-91, Mo-95, Mo-97, Ru-99, Rh-103, Pd-105, Cd-11, Sn-115, Te-125, I-127, Ba-135, Ba-137, Xe-129, Xe-131, Nd-145, Gd-155, Dy-161, Er-167, Yb-171, W-183, Os-187, Pt-195, Hg-199, Tl-205, Pb-207, Pt-198, and H-2. In some embodiments, the therapeutic microsphere lacks one or more of strontium phosphate, phosphate, or phosphorous. In some embodiments, the therapeutic microsphere does not comprise a strontium phosphate and/or phosphate layer over a substrate into which a therapeutic isotope is bound. In some embodiments, the substrate is not organic, lacks organic materials, and/or is not a resin. In some embodiments, the therapeutic radioisotope is not bound to the substrate by any one of a carboxylic acid group, a diphosphonic acid group, or a sulfonic acid group.

### Methods of Manufacturing and Products Made Therefrom

Some embodiments pertain to a therapeutic radioisotopic particle (or a population comprising therapeutic particles) made by a method that includes obtaining a particle (e.g., a substrate in the form of a particle) as disclosed elsewhere herein. As disclosed herein, in some embodiments, the particle comprises a substrate material. In some embodiments, the substrate material comprises Lewis basic constituents about a surface of the substrate (and/or throughout the substrate). In some embodiments, a radioisotope or a precursor thereof is combined with the substrate and a chemical bond between the substrate of the particle and the radioisotope or a precursor thereof is formed. In some embodiments, a radioisotope (e.g., therapeutic or a precursor thereof) is combined with the substrate and a chemical bond between a bridging atom and the substrate of the particle is formed. In some embodiments, a chemical bond between a non-metal atom and the radioisotope is also formed to provide a bridge from the radioisotope to the substrate. In some embodiments, the constituent atoms of the bridge are bound to each other chemically (e.g., through coordinate and/or covalent bonding).

Some embodiments pertain to a therapeutic radioisotopic microsphere (or a population comprising therapeutic particles) made by a method comprising providing a substrate comprising an inorganic material comprising a metalloid or a metal. In some embodiments, the core of the substrate comprises a first portion of metalloid or metal atoms bonded to the non-metal atoms, while the surface comprising a second portion of the metalloid or metal atoms bonded to the non-metal atoms. In some embodiments, the substrate comprises a therapeutic radioisotope or a precursor to a therapeutic radioisotope. In some embodiments, the substrate does not comprise a therapeutic radioisotope. In some embodiments, as disclosed elsewhere herein, the therapeutic radioisotope is bound directly to the substrate (e.g., through at least a portion of the non-metal atoms at the surface of the substrate), via a bridging metal atom through chemical bonds, or both. In some embodiments, the method further comprises obtaining at least one therapeutic radioisotope or a precursor thereto. In some embodiments, the method further comprises chemically coupling the at least one therapeutic radioisotope or precursor thereto to the surface layer of the substrate to provide the therapeutic particle or a particle with a precursor to a therapeutic isotope. In some embodiments, where precursors to the therapeutic radioisotope are provided, those precursors can be activated after or before functionalization of the microsphere with the radioisotope or precursor thereto. In some embodiments, a population of particles can be functionalized with multiple different therapeutic radioisotope is added simultaneously, consecutively, or separately (e.g., where a population comprising differing therapeutic particles is formed by mixing the particles).

In some embodiments, as disclosed elsewhere herein, the therapeutic radioisotope may be provided within the substrate (e.g., embedded). In some embodiments, additionally or alternatively, the therapeutic radioisotope may be bound directly to the substrate through at least a portion of the non-metal atoms at the surface of the substrate. In some embodiments, the method further comprises obtaining the at least one therapeutic radioisotope. In some embodiments, the method further comprises chemically coupling the at least one therapeutic radioisotope to the surface layer of the substrate to provide the therapeutic radioisotopic microsphere.

In some embodiments, the method comprises providing a precursor to the therapeutic radioisotope or an active therapeutic radioisotope in ionic form such as in the form of a salt prior to chemically coupling the precursor to the therapeutic radioisotope or active therapeutic radioisotope to the surface of the substrate. In some embodiments, the salt has an oxidation number of 1, 2, 3, 4, or 5. In some embodiments, the salt has one or more counter ions associated with it. In some embodiments, the counter ion has an oxidation number of −1 or −2. In some embodiments, the salt is an alkali metal salt, an alkali earth metal salt, a halogen salt, or a polyatomic salt.

In some embodiments, the chemical functionalization (e.g., of the therapeutic radioisotope, the precursor to the therapeutic radioisotope, or combinations thereof) is carried out in the presence of a reducing agent. In some embodiments, the substrate is contacted with the radioisotope in the presence of a reducing agent. In some embodiments, the reducing agent is selected from one or more of a tin salt (such as a stannous salt to provide stannous ions), HCl, sodium borohydride, sodium diothionite, ferrous sulfate, ferric chloride plus ascorbic acid, hypophosphorus acid, and/or hydrazine.

In some embodiments, the therapeutic radioisotope may be any therapeutic radioisotope as disclosed elsewhere herein. In some embodiments, a therapeutic radioisotopic particle is prepared which is thereafter functionalized with a therapeutic radioisotope. In other embodiments, a therapeutic radioisotopic particle is prepared which is thereafter functionalized with a therapeutic radioisotope.

In some embodiments, the therapeutic ceramic microsphere is obtainable by reacting the ceramic microsphere with a ⁹⁹mTc, which may for example be in the form of a pertechnetate ion, in the presence of a reducing agent, such as a stannous ion (for example as a stannous halide such as stannous chloride).

Some embodiments pertain to a process for the preparation of a therapeutic radioisotopic comprising reacting a ceramic microsphere substrate (e.g. comprising at least one non-metal, metalloid, or transition metal oxide) with ^{99m}Tc ions, such as ^{99m}Tc pertechnetate or other Tc(VII) ions in the presence of a reducing agent as described elsewhere. The ceramic microsphere may be in the form of a glass microsphere.

Some embodiments pertain to a process for the preparation of a therapeutic radioisotopic comprising reacting a ceramic microsphere substrate with a zirconium salt, such as ⁸⁹Zr oxalate or ⁸⁹Zr chloride. The reaction may be carried out in the presence of a base.

Some embodiments pertain to a method of making a therapeutic and/or therapeutic particle comprising providing the inorganic substrate and chemically functionalizing the inorganic substrate with the at therapeutic radioisotope or precursor to the therapeutic radioisotope to provide the therapeutic particle.

Some embodiments pertain to a method of making a therapeutic radioisotopic particle. In some embodiments, the method includes a step for providing therapeutic radioisotope as a salt prior to chemically functionalizing the at least one therapeutic radioisotope to a surface of the substrate. In some embodiments, salt is an alkali metal salt, an alkali earth metal salt, a halogen salt, or a polyatomic salt. In some embodiments, the method comprises adding a reducing agent during the chemical functionalization step. In some embodiments, the reducing agent is selected from one or more of a tin salt (such as stannous salt, such as a stannous halide, to provide stannous ions), HCl, sodium borohydride, sodium diothionite, ferrous sulfate, ferric chloride plus ascorbic acid, hypophosphorus acid, and/or hydrazine. In some embodiments, the reducing agent is one that is capable of reducing Tc(VII) to Tc(V).

As disclosed elsewhere herein, a precursor to the therapeutic radioisotope or an active therapeutic radioisotope may be provided on or in the microsphere. These precursors or active radioisotopes may be added to the microsphere and/or activated in any order. For example, in some embodiments, the particle is provided having a substrate with a precursor to a therapeutic radioisotope embedded therein. In some embodiments, an active (e.g., radioactive and/or therapeutic) therapeutic radioisotope can be bound to the substrate to provide a therapeutic microsphere. In several embodiments, a precursor therapeutic radioisotope can be activated (e.g., through neutron bombardment) after the addition of the precursor radioisotope to the particle to provide a therapeutic radioisotopic microsphere. In other embodiments, the therapeutic radioisotope can be activated (e.g., through neutron bombardment) prior to the addition of the active radioisotope.

In several embodiments, the radioisotope is added to the substrate (e.g., the microsphere) by adding the radioisotope (or a salt thereof) to a solution comprising the substrate. In several embodiments, the radioisotope is added to the substrate (e.g., the microsphere) by adding substrate to a solution comprising the radioisotope (or a salt thereof). In several embodiments, the solution comprises water. In several embodiments, the solution comprises saline. In several embodiments, the solution has a pH of greater than or equal to about: 3.0, 4.0, 5.0, 5.5, 6.0, 6.5, 7.0, 7.5, 8.0, 9.0, 10.0, or ranges spanning and/or including the aforementioned values. In several embodiments, the solution comprises a buffer. In several embodiments, the buffer is selected from the group consisting of phosphate buffered saline (PBS), citrate, acetate, or combinations of the foregoing. In several embodiments, the solution lacks a buffer. Suitable pH ranges include pH 3 to pH 10, and pH 5 to pH 8.

### Methods of Using Therapeutic Radioisotopic Particles

As disclosed elsewhere herein, in some embodiments, a method of using therapeutic radioisotopic particles (or a population comprising therapeutic particles) is provided. For example, as noted above, in SIRT, therapeutic radioactive particles as disclosed herein may be introduced into a blood vessel of the body of a patient through a catheter. These therapeutic particles can be used for the treatment of vascularized tumors such as liver cancer (e.g., hepatic neoplasias such as hepatocellular carcinoma (HCC), as well as tumors derived from metastasis of other tumors to the liver, such as neuroendocrine tumors and colorectal tumors), as well as those of the brain, prostate, lung, spleen and kidney, for example. In some embodiments, the therapeutic radioisotopic particles (or a population comprising therapeutic particles) as disclosed herein can be used in methods of treating vascularized tumors having a vascular supply (e.g., cancerous or benign tumors), such as those found in liver cancer (e.g., hepatic neoplasias such as hepatocellular carcinoma (HCC), as well as tumors derived from metastasis of other tumors to the liver, such as neuroendocrine tumors and colorectal tumors), as well as those of the brain, prostate, lung, spleen and kidney.

Some embodiments pertain to a method of determining a dose of radiation from therapeutic radioisotopic particles (e.g., microspheres) at a target site for treatment in a patient. In some embodiments, as disclosed elsewhere herein, a population of therapeutic radioisotopic particles (or a population comprising therapeutic particles) is administered to the patient. In other embodiments, the population can comprise particles having a plurality of different radioisotope types (e.g., 2, 3, 4, or more). For example, in some embodiments, only a single radioisotope (e.g., ⁹⁹mTc) and a single therapeutic radioisotope (e.g., ⁸⁹Sr) is present in the population of particles. In other embodiments, multiple types of therapeutic isotopes (e.g., ⁸⁹Sr and ²¹²Bi) are present in the population of particles.

In some embodiments, a dose of therapeutic radioisotopic microspheres (or a population comprising therapeutic particles) that is estimated to be sub-therapeutic (e.g., through a calculation on a mg particle/kg body weight ratio) are introduced to the patient. In other embodiments, a dose of therapeutic radioisotopic microspheres (or a population comprising therapeutic particles) that is estimated to be therapeutic (e.g., through a calculation on a mg particle/kg body weight ratio) are introduced to the patient. In some embodiments, the estimated sub-therapeutic dose or estimated therapeutic dose of therapeutic radioisotopic particles (or a population comprising therapeutic particles) are allowed to distribute within the patient over a period of time.

In some embodiments, after injecting the population of surrogate particles (or a population comprising therapeutic particles) into the patient (e.g., by a transcatheter, etc.), the particles are allowed to distribute within the body of the patient for a period of time. In some embodiments, the therapeutic radioisotopic particles (or a population comprising therapeutic particles) are allowed to distribute within the body for a period of time that is less than or equal to about: 5 minutes, 10 minutes, 15 minutes, 30 minutes, an hour, or ranges including and/or spanning the aforementioned values. When nanoparticles are used in a passive delivery route (as disclosed elsewhere herein), the particles may be allowed to distribute within the body for a longer period of time, such as equal to or at least about: an hour, two hours, 6 hours, 8 hours, 24 hours, or ranges including and/or spanning the aforementioned values.

Target areas may include cancer tumors or benign tumors in a patient in need of treatment. Target areas may include vascularized tumors (e.g., cancerous or benign tumors) such as those found in liver cancer (e.g., hepatic neoplasias such as hepatocellular carcinoma (HCC), as well as tumors derived from metastasis of other tumors to the liver, such as neuroendocrine tumors and colorectal tumors), as well as those of the brain, prostate, lung, spleen and kidney, for example.

Some embodiments pertain to using different administration techniques to achieve distribution of the particles within the body. For example, a particular administration route can be selected depending on the size of the particle used. When microscale particles (e.g., microspheres) are used, systemic distribution or passive may not be appropriate because these particles tend to become lodged within capillary networks and not readily distribute in distal areas of the body away from the site of the injection. When microscale particles are used, these particles may be injected in a catheter directed way (e.g., site-directed). In some embodiments, the particles are injected into a blood vessel that directly feeds a target tissue. For example, catheter introduction at a renal artery may be selected to treat a renal tumor. Catheter introduction at a hepatic artery may be selected to treat a liver tumor. Where needed, as disclosed elsewhere herein, additional particles can be introduced to the target site. Alternatively, if a therapeutic dose would cause intolerable or excessive damage to off-target areas of the patient, the patient can be withdrawn from treatment.

An additional method of injection is directly into a tumor or a target tissue. This injection technique can be performed using nanoscale or microscale particles. In this introduction method, the particles are injected and remain relatively stationary at the injection site, where they have their therapeutic effect. Where needed, additional particles can be introduced to the target site.

When nanoscale particles are used, systemic (e.g., i.v. delivery) may be used. In some embodiments, the methods involve injecting (e.g., infusing) nanoparticles into the patient systemically. Systemic introduction can involve the introduction of nanoparticles into the circulatory system and/or at a site within the body but away from or remote from the target site. These sites can be located in areas of the patient's body that are not proximally located to the site of treatment (e.g., at sites in the body other than the tumor site). For instance, nanoparticles may accumulate preferentially within tumors as a result of their size and passive extravasation from the leaky, chaotic and immature vasculature of tumors; a phenomenon referred to as the “enhanced permeability and retention” (EPR) effect. In some embodiments, after being introduced to the body, the nanoparticles are allowed to passively accumulate at a tumor. In some embodiments, this passive accumulation is achieved by the passage of a defined period of time. In some embodiments, the nanoparticles are allowed to accumulate in the tumor for a period of time ranging from between equal to or at least about 4 hours and/or less than or equal to about 36 hours after infusion and/or injection. In some embodiments, the nanoparticles are allowed to accumulate in the tumor site for a period of at least: about 4 hours, about 8 hours, about 12 hours, 24 hours, about 36, or ranges including and/or spanning the aforementioned values. Where needed, additional particles can be introduced to the target site. Alternatively, if a therapeutic dose would cause intolerable or excessive damage to off-target areas of the patient, the patient can be withdrawn from treatment.

Some embodiments of nanoparticles as disclosed herein further contain a targeting moiety linked to the particles, where the targeting moiety is exposed on an outer surface of the nanoparticle. In some embodiments, the targeting moiety is a nucleic acid, a small molecule, or a polypeptide. In some embodiments, the small molecule is a carbohydrate or hydrocarbon. In some embodiments, the polypeptide is selected from the group of: a growth factor, a hormone, a cytokine, an interleukin, an antibody, an antigen-binding antibody fragment, an integrin, a fibronectin receptor, a P-glycoprotein receptor, a peptidomimetic, an affibody, a nanobody, an avimer, a small modular immunopharmaceutical, and an adnectin, or a fragment thereof. In some embodiments, the targeting moiety is a member of the MUC-type mucin family, epidermal growth factor (EGFR) receptor, carcinoembryonic antigen (CEA), human cancer antigen, vascular endothelium Growth factor (VEGF) antigen, melanoma antigen (MAGE) gene, family antigen, T/Tn antigen, hormone receptor, growth factor receptor, cluster designation/differentiation (CD) antigen, tumor suppressor gene, cell cycle Regulatory factor, oncogene, oncogene receptor, proliferation marker, adhesion molecule, proteinase involved in degradation of extracellular matrix, malignant transformation related factor, apoptosis related factor, human cancer antigen, glycoprotein antigen, DF3, 4F2, MGFM antigen Breast tumor antigen CA15-3, calponin, cathepsin, CD31 antigen, proliferating cell nuclear antigen 10 PC 10) and may be selected from cell surface markers such as pS2. In the case of other forms of cancer and its metastases, one specific marker or multiple markers that may be used are, for example, vascular endothelial growth factor receptor (VEGFR) family, carcinoembryonic antigen (CEA) family member, anti-idiotype mAB, A kind of ganglioside mimicry, a member of cluster designation/differentiation antigen, a member of epidermal growth factor receptor (EGFR) family, a kind of cell adhesion molecule, a member of MUC type mucin family, a kind of cancer antigen (CA), a matrix metalloproteinase, a melanoma-associated antigen (MAA), a proteolytic enzyme, calmodulin, a member of the tumor necrosis factor (TNF) receptor family, an angiogenic factor marker, and a T cell (MART) antigen. In some embodiments, the targeting agent is selected from one or more of prostate membrane specific antigen (PMSA), small cell lung cancer antigen (SCLCA), T/Tn antigen, hormone receptor, tumor suppressor gene antigen, cell cycle regulator antigen, oncogene antigen. In some embodiments, the targeting agent is selected from one or more of cell surface markers such as oncogene receptor antigens, proliferation markers, proteinases involved in extracellular matrix degradation, malignant transformation-related factors, apoptosis-related factors, and one of human cancer antigens. In some embodiments, the targeting moiety specifically binds to prostate-specific membrane antigen. In some embodiments, the small molecule is S,S-2[3-][5-amino-1-carboxypentyl]-ureido]-pentanedioic acid (LIG).

Some embodiments pertain to a method for treating cancer (e.g., a cancer tumor) in a patient using therapeutic particles (or a population comprising therapeutic particles). In some embodiments, a population of therapeutic radioisotopic particles (or a population comprising therapeutic particles) (e.g., microspheres or microparticles) is provided to the patient in a feeder artery to the organ or tissue having the cancer tumor. In other embodiments, a population of therapeutic radioisotopic particles (or a population comprising therapeutic particles) (e.g., nanoparticles with or without targeting agents decorating the particle) is provided to the patient systemically. In some embodiments, the population of therapeutic radioisotopic particles (or a population comprising therapeutic particles) is delivered to the patient by introducing the population to a first position in a vasculature of a body of the patient. In some embodiments, the population is allowed to distribute within the body of the patient. In some embodiments, the method includes administering to the patient the additional amount of particles. In some embodiments, the target section of the body is a liver of the patient. In some embodiments, the target section of the body is a brain of the patient. In some embodiments, the target section of the body is a lung of the patient. In some embodiments, the target section of the body is the prostate of the patient. In some embodiments, the target section of the body is a kidney of the patient. In some embodiments, the target section of the body is a spleen of the patient. In some embodiments, the target section of the body is the gastrointestinal tract of the patient. In some embodiments, the target section of the body is the pancreas of the patient. In some embodiments, the target section of the body is the adrenal gland of the patient. In some embodiments, the target section of the body is the gallbladder of the patient. In some embodiments, the target section of the body is the bladder of the patient. In some embodiments, the target section of the body is muscle of the patient. In some embodiments, the target section of the body is bone of the patient. In some embodiments, the target section of the body is the thyroid of the patient. In some embodiments, the target section of the body is an ovary of the patient. In some embodiments, the target section of the body is the uterus of the patient. In some embodiments, the method of treatment includes treating a tumor in any of the aforementioned target sections.

Some embodiments pertain to a method for treating liver cancer in a patient using therapeutic particles (or a population comprising therapeutic particles). In some embodiments, a population of therapeutic radioisotopic particles (or a population comprising therapeutic particles) is provided to the patient in a hepatic feeder artery. In some embodiments, the population is delivered to the patient by introducing the population to a first position in a vasculature of a body of the patient. In some embodiments, the population is allowed to distribute within the body of the patient. In some embodiments, the method includes administering to the patient the additional amount of therapeutic radioisotopic particles (or a population comprising therapeutic particles).

In some embodiments, the target section of the body is a portion of the body having a tumor. In some embodiments, the target section of the body is a portion of the body to be treated and or having a tumor to be treated in a method of treatment. In some embodiments, the target section of the body is a liver of the patient. In some embodiments, the target section of the body is a brain of the patient. In some embodiments, the target section of the body is a lung of the patient. In some embodiments, the target section of the body is the prostate of the patient. In some embodiments, the target section of the body is a kidney of the patient. In some embodiments, the target section of the body is a spleen of the patient. In some embodiments, the target section of the body is the gastrointestinal tract of the patient. In some embodiments, the target section of the body is the pancreas of the patient. In some embodiments, the target section of the body is the adrenal gland of the patient. In some embodiments, the target section of the body is the gallbladder of the patient. In some embodiments, the target section of the body is the bladder of the patient. In some embodiments, the target section of the body is muscle of the patient. In some embodiments, the target section of the body is bone of the patient. In some embodiments, the target section of the body is the thyroid of the patient. In some embodiments, the target section of the body is an ovary of the patient. In some embodiments, the target section of the body is the uterus of the patient. In some embodiments, the method of treatment includes treating a tumor in any of the aforementioned target sections.

In some embodiments, at the time of injection, a dose of microspheres having a radioactivity intensity of equal to or less than about 50 microcuries (μCi), 100 μCi, 150 μCi, 250 μCi, 1000 μCi, 2000 μCi, or 4000 μCi, (or ranges including and/or spanning the aforementioned values) is injected. In some embodiments, a dose of 10 mg to 100 mg of microspheres is injected. In some embodiments, to the subject is administered a dose of less microspheres (in mg) of less than or equal to about: 5 mg, 10 mg, 25 mg, 50 mg, 75 mg, 100 mg, or ranges including and/or spanning the aforementioned values.

### Kits and Methods of Use Thereof

Some embodiments pertain to a kit comprising the therapeutic radioisotopic particles as disclosed herein.

In some embodiments the kit comprises the underivatized particles as described herein and instructions for carrying out the methods described herein to react the therapeutic radioisotope (or precursors thereof) with the particles.

In some embodiments, a kit comprises a particle (e.g., nanoparticle, microparticle, microsphere, etc.) comprising a substrate comprising an inorganic material that comprises metalloid or metal atoms bonded to non-metal atoms, the substrate comprising: a core extending to a surface, the core comprising a first portion of the metalloid or metal atoms bonded to the non-metal atoms and the surface comprising a second portion of the metalloid or metal atoms bonded to the non-metal atoms. In some embodiments, the kit comprises instructions for reacting a therapeutic radioisotope with the substrate such as to bind the therapeutic radioisotope directly to the substrate through at least a portion of the non-metal atoms at the surface of the substrate.

In some embodiments, a kit comprises a particle (e.g., microsphere) comprising: an inorganic substrate; wherein the inorganic substrate comprises at least one non-metal and at least a metalloid or a metal; and instructions for binding a therapeutic radioisotope to the surface of the inorganic substrate through a Lewis acid-base coordination bond.

In some embodiments a kit comprises a particle (e.g., microsphere) comprising a ceramic microsphere substrate and instructions for carrying out a reaction in which a therapeutic radioisotope is coupled to the ceramic substrate. In some embodiments the instructions are for carrying out a reaction in which a therapeutic radioisotope is coupled to the ceramic particle (e.g., microsphere) substrate as a Lewis acid base adduct of an inorganic base.

In some embodiments the kit comprises 50 μl to 2 ml of particles (e.g., microspheres) by packed volume, in a sealed unit. In some embodiments the sealed unit may be a container such as a vial, e.g. a glass vial, in other embodiments the sealed unit may be a syringe. The particles (e.g., microspheres) may be provided sterile.

In some embodiments the kit may additionally comprise a reducing agent.

In some embodiments, the kit comprises instructions for using a catheter to introduce the therapeutic radioisotopic particles into a patient. In some embodiments, the kit contains one or more of a vascular access needle, a vascular guidewire, a vascular sheath (e.g., 4-6Fr), a vascular catheter (4-5Fr), a microcatheter, syringes, and a vial.

In some embodiments, an therapeutic radioisotopic particle Administration Set is acquired. In some embodiments, the set comprises a sterile disposable tubing set and one empty sterile vial. In some embodiments, the tubing set is made of pre-assembled, sterile components and is for single use only. In some embodiments, the pre-assembled tubing set contains a needle plunger assembly and an integrated 20 cc syringe. In some embodiments, the one way valves incorporated in the administration set control the flow of liquid such that it will only flow in the appropriate direction. In some embodiments, the pulling back on the syringe plunger will fill the syringe from the fluid source. In some embodiments, the pushing the syringe plunger will move fluid toward the needle plunger assembly. In some embodiments, prior to the infusion, the Administration Set is manually pre-primed by pushing the sterile flushing solution through the set to purge air from the lines.

In some embodiments, the Administration Accessory Kit is acquired. In some embodiments, the Administration Accessory Kit contains re-usable accessories including one or more of an acrylic box base, top shield, removable side shield and bag hook. In some embodiments, the Administration Accessory Kit facilitates monitoring of the infusion process and provides beta radiation shielding. In some embodiments, the Administration Accessory Kit should be placed on a sturdy cart or table that is positioned beside the patient, close to the infusion catheter inlet luer fitting. In some embodiments, an extension arm on the Accessory Kit facilitates alignment and positioning of the Administration Set/patient catheter connection.

In some embodiments, throughout the administration procedure, a therapeutic radioisotopic particle dose vial remains sealed within the clear acrylic vial shield in which it is supplied. In some embodiments, the removable plug at the top of the acrylic vial shield provides access to the septum of the radioisotopic particle dose vial. In some embodiments, the needle plunger assembly is designed to snap into the top of the acrylic shield, and is not easily removed once snapped into place. In some embodiments, this provides stability and alignment for the needles which are inserted through the septum when the tabs are pushed down on the plunger assembly.

In some embodiments, the constant syringe pressure should be maintained for the duration of each flush, with a flow rate equal to or greater than 20 cc per minute. One flush is 20 cc as indicated on the barrel of the syringe. In some embodiments, using a flow rate of less than 20 cc per minute (i.e. appropriate to the flow of the native vessel) may decrease the delivery efficiency of the administration system. In some embodiments, flushing should be continued until optimal delivery of radioisotopic particle is achieved. In some embodiments, a minimum of three flushes for a total of 60 cc is recommended. In some embodiments, the infusion pressure should not exceed 30 psi on any flush. In some embodiments, the pressure relief valve in the Administration Set has been included to prevent over pressurization.

In some embodiments, in order to minimize the potential of a high radiation hand dose, use a hemostat, forceps, or towels/gauze when handling parts of the Administration Set after infusion. In some embodiments, before administration, the acrylic shield containing the dose is measured at a distance of 30 cm from the detector.

Some embodiments provide a sealed unit containing 50 μl to 2 ml of the underivatized particles (e.g., microspheres) described herein by packed volume. The sealed unit may be a vial or a syringe for example.

In some embodiments the underivatized particles (e.g., microspheres) comprise a substrate comprising an inorganic material that comprises metalloid or metal atoms bonded to non-metal atoms, the substrate comprising: a core extending to a surface, the core comprising a first portion of the metalloid or metal atoms bonded to the non-metal atoms and the surface comprising a second portion of the metalloid or metal atoms bonded to the non-metal atoms.

In some embodiments the underivatized particles (e.g., microspheres) comprise a substrate comprising an inorganic material that comprises metalloid or metal atoms bonded to non-metal atoms, the substrate comprising: a core extending to a surface, the core comprising a first portion of the metalloid or metal atoms bonded to the non-metal atoms and the surface comprising a second portion of the metalloid or metal atoms bonded to the non-metal atoms as described herein.

In some embodiments the underivatized particles (e.g., microspheres) comprise a ceramic particle (e.g., microsphere) substrate as described herein. The particles (e.g., microspheres) may be provided sterile.

Some embodiments pertain to a kit. In some embodiments, the kit comprises a particle (e.g., microsphere) comprising a substrate comprising an inorganic material that comprises metalloid or metal atoms bonded to non-metal atoms. In some embodiments, the substrate comprises a core extending to a surface, the core comprising a first portion of the metalloid or metal atoms bonded to the non-metal atoms and the surface comprising a second portion of the metalloid or metal atoms bonded to the non-metal atoms and instructions for reacting a therapeutic radioisotope with the substrate such as to bind the radioisotope directly to the substrate through at least a portion of the non-metal atoms at the surface of the substrate.

In some embodiments, the kit comprises a particle (e.g., microsphere) comprising a substrate; wherein the substrate comprises at least one non-metal, a metalloid, or a transition metal oxide and instructions for binding a therapeutic radioisotope to the surface of the substrate through a Lewis acid-base coordination bond.

In some embodiments, the kit comprises a particle (e.g., microsphere) comprising a ceramic particle (e.g., microsphere) substrate and instructions for carrying out a reaction in which a therapeutic radioisotope is coupled to the ceramic particle (e.g., microsphere) substrate as a Lewis acid base adduct.

In some embodiments, the kit comprises 50 μl to 2 ml of particles (e.g., microspheres) by packed volume, in a sealed unit. In some embodiments, the particles (e.g., microspheres) are provided in a vial or a syringe.

In some embodiments, the radioisotope included in any kit as disclosed herein is selected from ¹⁷⁷Lu, ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, Al¹⁸F, and/or combinations thereof. In some embodiments, the therapeutic radioisotope included in any kit as disclosed herein is selected from ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb, and/or combinations thereof.

In some embodiments, the kit comprises instructions for carrying out a reaction in which the therapeutic radioisotope, the precursor to the therapeutic radioisotope, or combinations thereof are coupled to the substrate as a Lewis acid base adduct.

In some embodiments, the kit comprises instructions for activating a precursor to a therapeutic isotope within the substrate to provide a therapeutic isotope. In some embodiments, the instructions for activating the precursor to the therapeutic isotope within the substrate indicate that the precursor to the therapeutic isotope is activated prior to coupling the radio isotope to the substrate.

In some embodiments, the kit comprises instructions for activating a precursor to a therapeutic isotope and/or a precursor to a therapeutic radioisotope at the surface of the substrate to provide a therapeutic isotope. In other embodiments, the instructions for activating the precursor indicate that the precursor to the isotope is activated prior to coupling the to the substrate. In some embodiments, the radioisotope included in any kit as disclosed herein is selected from ¹⁷⁷Lu, ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, Al¹⁸F, and/or combinations thereof. In some embodiments, the therapeutic radioisotope is one or more of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

In some embodiments, the kit additionally comprises a reducing agent. In some embodiments, the reducing agent is selected from one or more of a stannous salt, concentrated HCl, sodium borohydride, sodium diothionite, ferrous sulfate, ferric chloride plus ascorbic acid, hypophosphorus acid, and/or hydrazine. In some embodiments, the reducing agent is a stannous salt, the radioisotope is ⁹⁹mTc and the radioisotope is in the form of a pertechnetate salt.

In some embodiments, the radioisotope is ⁸⁹Zr. In some embodiments, the radioisotope is in the form of ⁸⁹Zr zirconium oxalate.

In some embodiments, the radioisotope is ¹⁷⁷Lu.

In some embodiments, the substrate comprises a yttrium oxide aluminosilicate.

In some embodiments, the kit further comprises one or more of a vascular access needle, a vascular guidewire, a vascular sheath (e.g., 4-6Fr), a vascular catheter (4-5Fr), a microcatheter, syringes, and a vial.

### EXAMPLES

The following examples provide illustrations of some embodiments disclosed herein and are not intended to be limiting. One skilled in the art will appreciate readily that the present disclosure is well adapted to carry out the objects and obtain the ends and advantages mentioned, as well as those objects, ends and advantages that flow naturally from the embodiments disclosed herein. Changes therein and other uses which are characteristic attributes of the disclosure as defined by the scope of the claims will occur to those skilled in the art.

### Example 1: Preparation of Zirconium-89 Coupled Yttrium Aluminum Silicon Oxide Microspheres

A 4 mL glass vial was charged with yttrium aluminum silicon oxide glass beads. The Y₂O₃ was therefore in the form of naturally occurring ⁸⁹Y. The yttrium aluminum silicon oxide glass beads were suspended in 300 μL of deionized water. Next, a 2 μL aliquot of Zirconium-89 in 1 M oxalic acid (3D imaging, Little Rock, AK)) (about 100 microcuries (μCi)) was added to the reactor vial, followed by 2 μL of 2 M sodium carbonate, followed by a Teflon®-coated magnetic stir bar. The reaction mixture was stirred and heated at 120° C. for 2 hours on an aluminum heating block, then removed from the block and allowed to cool to room temperature. The yttrium aluminum silicon oxide glass microspheres were suspended in 3.0 mL of deionized water and passed through a 0.22 μm syringe filter to collect the microspheres for labeling analysis. The glass vial was rinsed with an additional 4 mL of deionized water, which was then passed through the syringe filter. The syringe filter (containing the labeled microspheres), the glass vial, and the deionized water filtrate were analyzed by a gamma well counter and the results are summarized in the Table 1 below.

TABLE 1

| | | Remaining | Deionized | Radio- |
|---|---|---|---|---|
| Mass of | Filter | in reactor | water | chemical |
| TheraSphere | (microspheres) | vial | filtrate | Yield |
| 100 mg | 118 μCi | 30 μCi | 4 μCi | 77% |
| 25 mg | 539 μCi | 310 μCi | 10 μCi | 63% |
| 25 mg* | 122 μCi | 363 μCi | 8 μCi | 25% |
| 10 mg | 46 μCi | 43 μCi | 10 μCi | 46% |
| 10 mg# | 78 μCi | 46 μCi | 6 μCi | 60% |

*Reactor vial was not charged with a Teflon-coated magnetic stir bar.

### Example 2: Preparation Of ⁹⁹mTc Coupled Yttrium Aluminum Silicon Oxide Microspheres

Tin(II) chloride dihydrate (1 mg) was added to a 1 dram vial and dissolved in 400 μL of deionized water. In a separate 1 dram vial, yttrium aluminum silicon oxide glass beads (⁸⁹Y, non-radioactive microspheres) were added, followed by 100 μL of [⁹⁹mTc]sodium pertechnetate (>30 mCi/mL). The tin(II) chloride dihydrate solution was added to the yttrium aluminum silicon oxide glass bead vial, briefly mixed (approximately 3 seconds) and the vial was capped and allowed to react at room temperature for 60 minutes. The microspheres were suspended in 3.0 mL of deionized water and passed through a 0.22 m syringe filter to collect the microspheres for labeling analysis. The glass vial was rinsed with an additional 4 mL of deionized water, which was then passed through the syringe filter. The syringe filter (containing the labeled microspheres), the glass vial, and the deionized water filtrate were analyzed by a gamma well counter and the results are summarized in the table below.

TABLE 2

| | | | Deionized | Radio- |
|---|---|---|---|---|
| Mass of | Filter | Remaining in | water | chemical |
| TheraSphere | (microspheres) | reactor vial | filtrate | Yield |
| 100 mg | 4.1 mCi | 0.26 mCi | 1.2 mCi | 73% |
| 25 mg | 1.65 mCi | 0.63 mCi | 0.25 mCi | 63% |
| 25 mg* | 2.28 mCi | 0.15 mCi | 0.1 mCi | 90% |
| 10 mg | 1.766 mCi | 0.244 mCi | 0.354 mCi | 75% |

All radiochemical yield (RCY) data are reported as mean of n = 3 runs.
*Reactor vial was not charged with a Teflon-coated magnetic stir bar.

### Example 3: [⁸⁹Zr] Microsphere Ligand Challenge with DFO Chelate

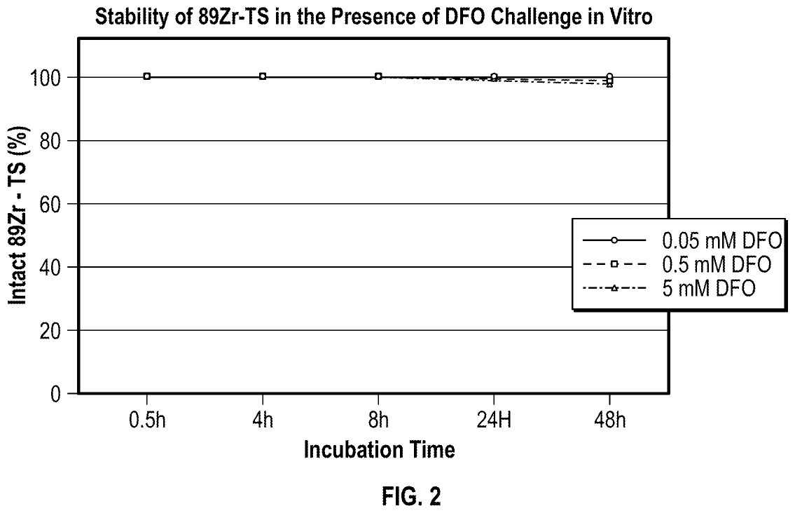

Yttrium aluminum silicon oxide glass beads (suspended in 200 μL sterile saline, pH 7-8) were mixed with increasing concentrations of desferrioxamine (DFO), ranging from 0.05 to 5 mM, and incubated at 37° C. under constant stirring. At each time point, 5 μL of solution was removed, added to a 0.45 μm spin filter, and diluted with 100 μL of deionized water. The spin filter was centrifuged at 13,200×G for 60 seconds, 100 μL of deionized water was added back to the spin filter and centrifugation repeated. The spin filter was removed from the microcentrifuge tube and the supernatant was analyzed by gamma spectroscopy, then counted in a gamma counter, to detect formation of possible ⁸⁹Zr-DFO. Negligible ⁸⁹Zr detachment was detected for ⁸⁹Zr-TS within 48 h, demonstrating a strong binding affinity of ⁸⁹Zr to TS. Results are shown in FIG. 2.

### Example 4: Test for Specificity of Tin (II) Chloride as a Reducing Agent

Several oxidants and reducing agents were tested on the premise of understanding the specificity of tin(II) chloride for reducing pertechnetate toward reaction with yttrium aluminum silicon oxide glass beads (microspheres). Tin is not only expected to actively reduce Tc(VII) to Tc(V), but seems to actively participate in the binding of ^{99m}Tc to yttrium aluminum silicon oxide glass beads. Several reducing agents were attempted to facilitate yttrium aluminum silicon oxide glass bead/^{99m}Tc coupling: FeCl₂/ascorbic acid (pH=2); sodium borohydride; and zinc metal. None of the reducing agents yielded substantial radiochemical yield's of ^{99m}Tc-yttrium aluminum silicon oxide glass beads and were not considered further. This suggests tin plays a role in the binding of ^{99m}Tc to yttrium aluminum silicon oxide glass beads.

### Example 5: Effect of Reaction Volume on the Yield of ^{99M}Tc Coupled Microspheres

Tin(II) chloride dihydrate (1 mg) was added to a 1 dram vial and dissolved in deionized water as indicated in Table 3. In a separate 1 dram vial, 10 mg of yttrium aluminum silicon oxide glass beads (microspheres) are added, followed by 100 μL of [⁹⁹mTc] sodium pertechnetate (>30 mCi/mL). The tin(II) chloride dihydrate solution was added to the yttrium aluminum silicon oxide glass bead vial, briefly mixed (approximately 3 seconds) and the vial was capped and allowed to react at room temperature for 60 minutes. The microspheres were suspended in 3.0 mL of deionized water and passed through a 0.22 m syringe filter to collect the microspheres for labeling analysis. The glass vial was rinsed with an additional 4 mL of deionized water, which was then passed through the syringe filter. The syringe filter (containing the labeled microspheres), the glass vial, and the deionized water filtrate were analyzed by a gamma well counter and the results are summarized in the table below.

TABLE 3

| | | | Deionized | Radio- |
|---|---|---|---|---|
| Reaction | Filter | Remaining in | water | chemical |
| volume (μL) | (microspheres) | reactor vial | filtrate | Yield |
| 100 | 0.44 mCi | 0.01 mCi | 0.003 mCi | 95% |
| 200 | 0.055 mCi | 0.015 mCi | 0.01 mCi | 75% |
| 500 | 0.033 mCi | 0.022 mCi | 0.026 mCi | 41% |

### Example 6: Coupling to Alternative Ceramic Microparticles

Examples 1 and 2 were repeated using silicon oxide and silicon aluminum oxide microparticles. Silicon-aluminum oxide microspheres were obtained from Steag Energo Mineral. Silica microspheres were purchased from EPRUI Biotech Co. Limited (Product #: EPRUI-SI-20), which consisted of SiO₂, as monodisperse microspheres having a diameter of 20 μm.

TABLE 4
⁸⁹Zr silicon oxide microparticle

| | | | Deionized | Radio- |
|---|---|---|---|---|
| | Filter | Remaining in | water | chemical |
| Mass | (microspheres) | reactor vial | filtrate | Yield |
| 25 mg | 138 μCi | 0 μCi | 0 μCi | quantitative |
| 10 mg | 394 μCi | 10 μCi | 0 μCi | >97% |

TABLE 5
^{99m}Tc silicon oxide microparticle

| | | | Deionized | |
|---|---|---|---|---|
| | Filter | Remaining in | water | Radiochemical |
| Mass | (microspheres) | reactor vial | filtrate | Yield |
| 25 mg | 3.1 mCi | 0.0 mCi | 0.01 mCi | quant. |
| 10 mg | 2.87 mCi | 0.02 mCi | 0.01 mCi | >98% |

TABLE 6
⁸⁹Zr silicon-aluminum oxide microparticle

| | | | Deionized | |
|---|---|---|---|---|
| | Filter | Remaining in | water | Radiochemical |
| Mass | (microspheres) | reactor vial | filtrate | Yield |
| 25 mg | 900 μCi | 0 μCi | 0 μCi | quant. |
| 10 mg | 401 μCi | 10 μCi | 0 μCi | quant. |

TABLE 7
^{99m}Tc silicon-aluminum oxide microparticle

| | | | Deionized | |
|---|---|---|---|---|
| | Filter | Remaining in | water | Radiochemical |
| Mass | (microspheres) | reactor vial | filtrate | Yield |
| 25 mg | 3.5 mCi | 0.0 mCi | 0.01 mCi | quant. |
| 10 mg | 3.2 mCi | 0.02 mCi | 0.01 mCi | quant. |

### Example 7: Test for Mutual Dependency of 99mTc and TheraSphere on Tin for Product Formation

Several reactions were performed with various conditions as shown below.

General reaction conditions using 10 mg of yttrium aluminum silicon oxide glass beads (microspheres), 0.5-1.0 mg of SnCl₂, 3 mCi of ^{99m}Tc, and 400 μL of deionized water.

General reaction conditions using 10 mg of yttrium aluminum silicon oxide glass beads (microspheres), 3 mCi of ^{99m}Tc, and 400 μL of deionized water. No SnCl₂.

General reaction conditions using 25 mg of yttrium aluminum silicon oxide glass beads (microspheres), 3 mCi of ^{99m}Tc, and 400 μL of deionized water. No SnCl₂.

General reaction conditions using 0.5-1.0 mg of SnCl₂, 3 mCi of ⁹⁹mTc, and 400 μL of deionized water. No yttrium aluminum silicon oxide glass beads (microspheres).

TABLE 8

| | | | Deionized | |
|---|---|---|---|---|
| | Filter | Remaining in | water | Radiochemical |
| Reaction | (microspheres) | reactor vial | filtrate | Yield |
| 1 | 2.1 mCi | 0.35 mCi | 0.23 mCi | 80% |
| 2 | 0.1 mCi | 0 mCi | 2.3 mCi | <1% |
| 3 | 0.1 mCi | 0.03 mCi | 2.3 mCi | <1% |
| 4 | 1.2 mCi | 0.2 mCi | 1.1 mCi | 48% |

As demonstrated by Reaction 4, there is an association between some oxidation state of tin and ⁹⁹mTc. However, in the conditions tested, the product degrades in solution over 120 minutes without yttrium aluminum silicon oxide glass beads (microspheres) also being present, which is evident in Reaction 1. Also, there is no reaction when ⁹⁹mTc is mixed with yttrium aluminum silicon oxide glass beads (microspheres) alone without tin being present. The combination of the three reagents produces a stable product, which is demonstrated in our in vitro stability assay below.

### Example 8: Assay for Solution Stability of a ^{99M}Tc and ⁸⁹Zr Coupled Yttrium Aluminum Silicon Oxide Spheres

### Preparation of Sample Solutions

Three separate solutions were prepared as follows: 1) Vial #1: 10 mL of PBS and either 3-5 mCi of ⁹⁹mTc-yttrium aluminum silicon oxide glass beads (microspheres) or 100-150 μCi of ⁸⁹Zr-yttrium aluminum silicon oxide glass beads (microspheres), mix for homogeneity; 2) Vial #2: 10 mL of goat serum, 4±1 mCi of ⁹⁹mTc-yttrium aluminum silicon oxide glass beads (microspheres) (suspended in 100 μL of PBS to facilitate transfer) or 125±25 μCi of ⁸⁹Zr-yttrium aluminum silicon oxide glass beads (microspheres), mix for homogeneity; 3) Vial #3: 10 mL of goat serum (other serums can be used, e.g., horse, goat, or other mammal), 4±1 mCi of ⁹⁹mTc-yttrium aluminum silicon oxide glass beads (microspheres) (suspended in 100 μL of PBS to facilitate transfer) or 125±25 μCi of ⁸⁹Zr-yttrium aluminum silicon oxide glass beads (microspheres), 100 μL of 0.1 M HCl (Check pH, should be below 4, if not, add more 0.1M HCl until <4. It is okay if you overshoot pH to between 1 and 3, just note final pH), mix for homogeneity. Each vial was incubated at 37° C. for 8 hours. From each vial: Remove 100 μL, centrifuge and aliquot 10 μL of the supernatant for activity measurement into a 1 dram vial or microcentrifuge tube. To determine any effect of time, samples were withdrawn at selected time points.

### Prepare Standards for Gamma Counter

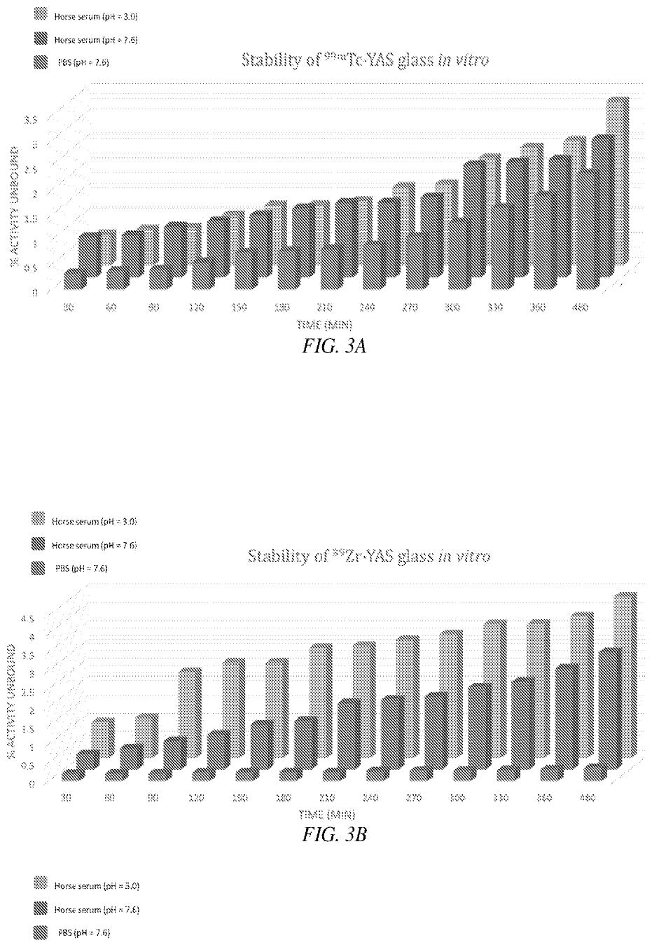

To a 100 mL volumetric flask was added 4±1 mCi of ^{99m}Tc-yttrium aluminum silicon oxide glass beads (microspheres) (suspended in 100 μL of PBS to facilitate transfer) or 125±25 μCi of ⁸⁹Zr-yttrium aluminum silicon oxide glass beads (microspheres). The solutions were diluted to 100 mL with deionized water and mixed. From the flask was removed 5×1 mL aliquots which were added into 5 separate vials. Stability data is shown in FIGS. 3A and 3B.

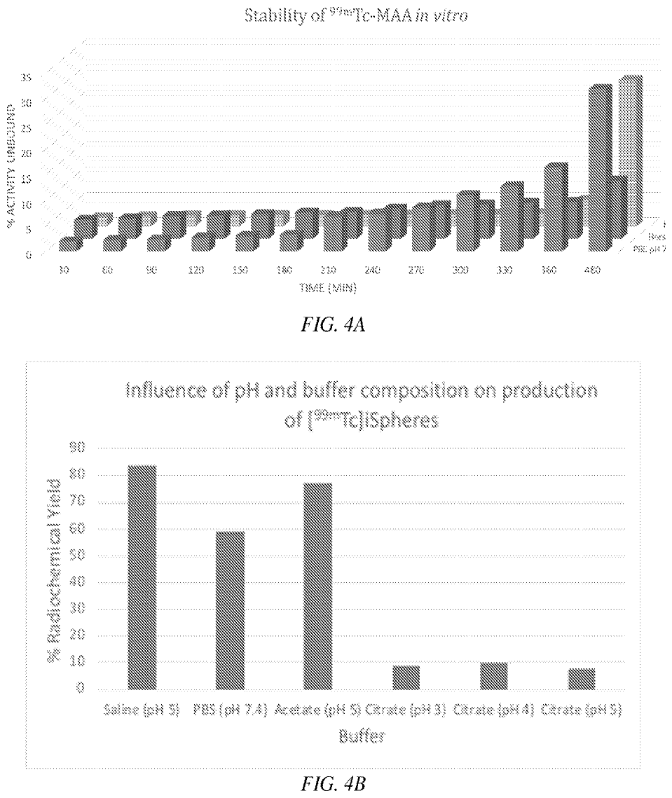

To compare the relative stability of ^{99m}Tc— and ⁸⁹Zr-yttrium aluminum silicon oxide glass beads (microspheres)against the industry standard in vitro, we performed the same serum assay using ^{99m}Tc-MAA. Three separate solutions were prepared as follows: 1) Vial #1 (Buffer): 10 mL of PBS, 4±1 mCi of ^{99m}Tc-MAA, mixed for homogeneity; 2) Vial #2 (Serum): 10 mL of goat serum, 4±1 mCi of ^{99m}Tc-MAA, mixed for homogeneity; 3) Vial #3 (Serum and Acid): 10 mL of goat serum; 4±1 mCi of ^{99m}Tc-MAA; 100 μL of 0.1 M HCl (Check pH, should be below 4, if not, add more 0.1M HCl until <4. It is okay if you overshoot pH to between 1 and 3, just note final pH), mixed for homogeneity. Each vial was incubated at 37° C. for 8 hours. From each vial: Remove 100 μL, centrifuge and aliquot 10 μL of the supernatant for activity measurement into a 1 dram vial or microcentrifuge tube. To determine any effect of time, samples were withdrawn at selected time points. Results are shown in FIG. 4.

The relative stabilities of all particles are summarized below in the following Tables.

TABLE 9
Relative solution stability of microspheres

| | 2 hr | 4 hr | 6 hr | 8 hr |
|---|---|---|---|---|
| PET-YAS | >99% | >99% | >99% | >99% |
| SPECT- | >99% | >99% | >98% | >97% |
| YAS | | | | |
| Tc-MAA | >97% | >93% | >84% | >68% |

TABLE 10
Relative serum stability of microspheres

| | 2 hr | 4 hr | 6 hr | 8 hr |
|---|---|---|---|---|
| PET-YAS | >99% | >98% | >97% | >96% |
| SPECT- | >99% | >98% | >97% | >97% |
| YAS | | | | |
| Tc-MAA | >97% | >94% | >92% | >88% |

### Example 9: Buffer and pH Studies

To a vial was added 0.5-1.0 mg of SnCl₂. 500 uL of buffer solution was added to the vial. The solution was filtered into a clean 1 dram vial. In a separate 1 dram vial (reaction vial), 10 mg of YAS microspheres were added along with 20 uL of Tc-99m stock solution. 380 uL SnCl₂ solution was added to the reaction vial, for a total reaction volume of 400 uL. The reaction vial was capped and mixed by hand for 3-5 seconds, then allowed to react at room temperature for 1 hr without stirring. The vial was then mixed the reaction mixture was pulled into a syringe with an 18 G×1.5″ needle. The labelled YAS microspheres were trapped on a 0.2 um syringe filter. A rinse reaction of the reaction vial was performed with 400 uL of DI H2O and the was trapped on the syringe filter.

Vial Labelling and Buffer List:
1. Saline (pH 5)
2. PBS (pH 7.4)
3. Acetate Buffer in Saline (pH 5)
4. Citrate Buffer (pH 3)
5. Citrate Buffer (pH 4)
6. Citrate Buffer (pH 5)

Analysis: Count the initial activity in the reaction vial (I), waste vial (W), Syringe Filter (SF), reaction vial (V), and note the background (BKG). Table 11 Provides the results.

TABLE 11

| Vial | I | W | SF |
|---|---|---|---|
| 1 | 2.70 mCi @ 1317 | 42 uCi @ 1327 | 2.27 mCi @ 1327 |
| 2a | 2.63 mCi @ 1328 | 118.6 uCi @ 1331 | 2.51 mCi @ 1330 |
| 2b | 1.96 mCi @ 1556 | 1.06 mCi @ 1558 | 726 uCi @ 1558 |
| 2c | 1.95 mCi @ 1600 | 870 uCi @ 1602 | 885 uCi @ 1603 |
| 3 | 2.60 mCi @ 1333 | 4 uCi @ 1336 | 2 mCi @ 1336 |
| 4 | 2.57 mCi @ 1337 | 2.17 mCi @ 1341 | 230 uCi @1341 |
| 5 | 2.58 mCi @ 1343 | 2.29 mCi @ 1346 | 263 uCi @1346 |
| 6 | 2.53 mCi @ 1348 | 2.30 mCi @1350 | 206 uCi @ 1350 |

| Vial | V | BKG |
|---|---|---|
| 1 | 420 uCi @ 1327 | 3 uCi @1328 |
| 2a | 124 uCi @ 1332 | 3 uCi @ 1332 |
| 2b | 174 uCi @ 1559 | 3.8 uCi @ 1559 |
| 2c | 197 uCi @ 1602 | 2.6 uCi @ 1603 |
| 3 | 642 uCi @ 1336 | 3 uCi @ 1337 |
| 4 | 10 uCi @ 1341 | 3 uCi @ 1342 |
| 5 | 12 uCi @ 1346 | 3 uCi @ 1347 |
| 6 | 37 uCi @ 1350 | 2.5 uCi @ 1351 |

As shown in FIG. 4B, buffering solutions may have a negative impact on the production of [99mTc] YAS microspheres. The best radiochemical yields were achieved using saline as the reaction solvent. Any attempt to buffer a solution with analyte and varying pH resulted in lower yields.

### Example 10: Prophetic Al¹⁸F Embodiment

Fluoride ion binds most metals, but forms particularly strong bonds with aluminum (III), which has been demonstrated historically to form complexes with metal-binding chelates; resulting in a highly stable (670 kJ/mol) Al—F bond. Aluminum forms octahedral complexes, thus, pentadentate coordination would be preferred for forming in vitro and in vivo stable 18F particles. [18F]fluoride is readily commercially available as an aqueous solution and thus, for practical use in a hospital setting, the reagents and reaction conditions should be compatible with aqueous reaction conditions. YAS glass (10 mg) and [18F]fluoride (supplied as an aqueous solution) are mixed with aluminum trichloride hydrate (monohydrate, hexahydrate, or other hydrated species to indicate compatibility with aqueous solutions) in pH 4 acetate buffer and an appropriate chelator (NOTA, NODA, trimethyltriazonane or other chelator species to support an octahedral aluminum-fluoride complex). The solution is heated at 100° C. for 15-30 minutes at which time the [18F]Al-YAS material is removed from heat and purified for use.

### Example 11: Prophetic Therapeutic Radioisotopic Particle Embodiment

This is a prophetic example. Therapeutic radioisotopic microspheres are prepared by activating precursor microspheres using neutron bombardment.

### Example 12: Prophetic Animal Study

Liver tumor bearing animals are used as models for studying diagnostic and therapeutic approaches for managing this condition. Woodchucks with liver tumors from chronic infection with Woodchuck Hepatoma Virus is an example of one such model. After catheterization of the proper hepatic artery of a liver tumor bearing Woodchuck with a microcatheter, therapeutic radioisotopic microspheres are administered. These distribute in a flow-directed fashion and lodge within the small arteries of the liver. The therapeutic radioisotopic microspheres are provided as a dose that will provide a lethal dose of therapeutic radioisotopic microspheres to liver tumor cells while minimizing injury to normal liver cells.

### Example 13: Using Therapeutic Particles

Based on the inventor's experience, the following prophetic results are projected using controlled studies. A group of suffering patients from liver cancer are selected for treatment by SIRT with therapeutic radioisotopic particles. Therapeutic microspheres are injected into the hepatic artery and allowed to distribute for a period of 15 minutes. A therapeutic dose of therapeutic radioisotopic particles that will provide 150 Gy (15000 rad) to the liver is administered. The microspheres have an average diameter of 20-30 m. The dose of the microspheres is dispersed in 0.6 mL pyrogen-free water.

### Example 14: In Vivo Animal Study

Radioembolization involves the endovascular delivery of particles with embedded radiation producing material through the arterial vasculature for the treatment of malignancy. A series of proof-of-concept experiments were performed at the University of Virginia to determine the distribution of ⁸⁹Zr labeled YAS microspheres. Though this example does not provide a therapeutic dose to study animals, it demonstrates that, were an alpha or beta emitter used and/or a sufficient quantity of radiation delivered using a therapeutic radioisotopic particle as disclosed herein, they would distribute in a flow directed fashion and therapeutic effect could be accomplished.

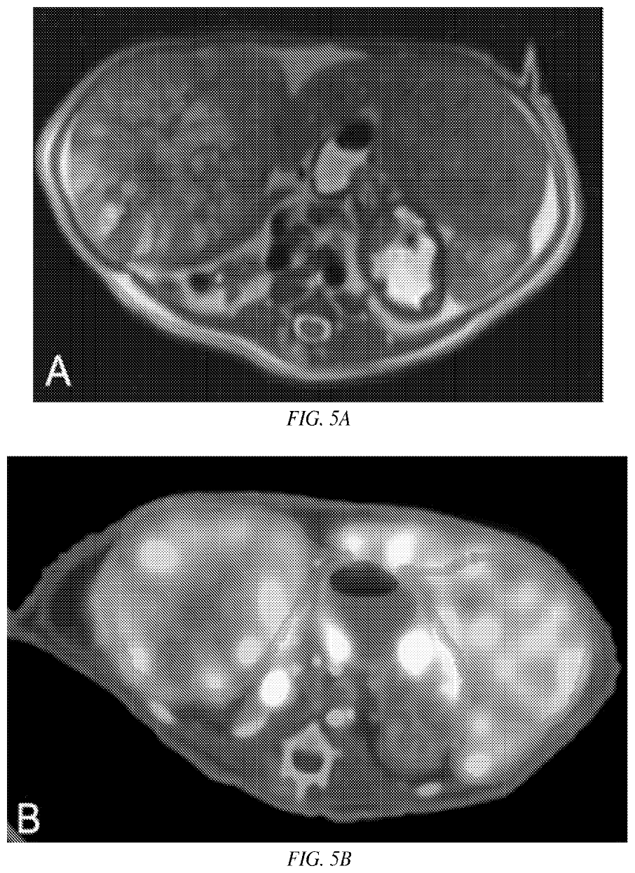

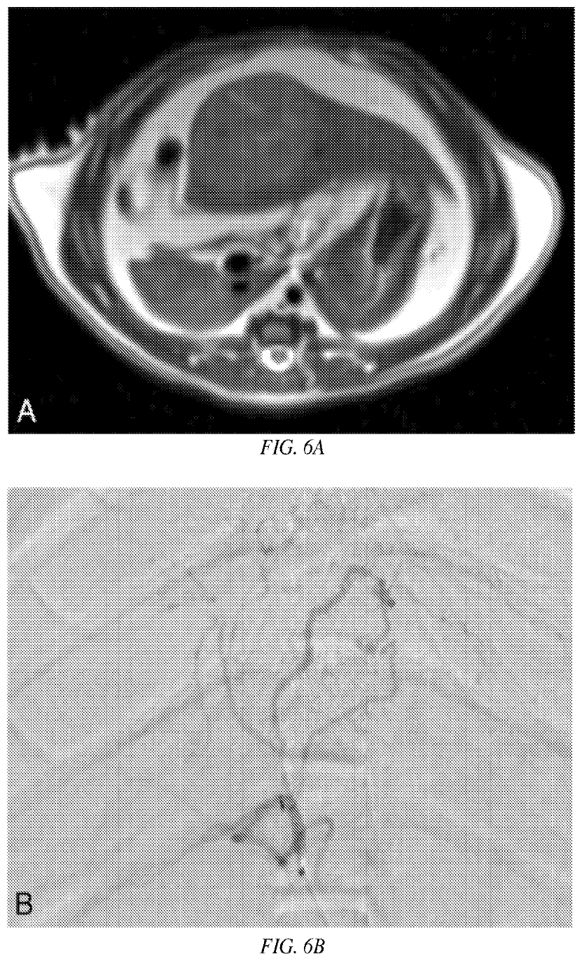

Prior to embolization, woodchucks were brought to a preparatory area and anesthesia was performed by veterinary staff. They administered ketamine (25-50 mg/kg) and xylazine (1-5 mg/kg) intramuscular. Atropine [0.04 mg/kg] was administered before intubation. The animal was intubated and maintained on a ventilator with isoflurane 1.5-2.5% in oxygen. The animal was placed on a heating pad to maintain body temperature. Animals underwent MRI (FIGS. 5A and 6A) on a Siemens 3 Tesla Prisma scanner (Erlangen, Germany) prior to angiography (FIG. 6B). For the MRI procedure, 1 mg/kg of pharmaceutical grade Magnevist was used.

Immediately following completion of the MRI (shown in FIGS. 5A and 6A), the animal was brought to the angiography suite. Ultrasound was used to gain access into the right common femoral artery using a 4F micropuncture kit (Cook Medical, Bloomington, IN). A 4F Glidesheath Slender Sheath (Terumo Medical, Somerset, NJ) was placed and a 4F angled tip catheter (Cook Medical, Bloomington, IN) was introduced over a wire into the abdominal aorta. Digital subtraction angiography (shown in FIG. 6B) was performed utilizing approximately 10 cc of Omnipaque 350 contrast media (GE Healthcare, Chicago, IL) to delineate the origin of the celiac artery. Next, a Headway Duo microcatheter (Microvention, Aliso Viejo, CA) was advanced into the hepatic arterial system with subsequent 3-5 cc injections of Omnipaque 350 contrast media performed to delineate supply to the tumor visualized on the previous ultrasound and MRI. The microcatheter was then positioned into the proper hepatic, left hepatic, or right hepatic artery for subsequent injection of ⁸⁹Zr labeled YAS microspheres. After establishing access to the intended delivery location, the catheter was secured in position and the animal was transferred under anesthesia to the PET/CT area.

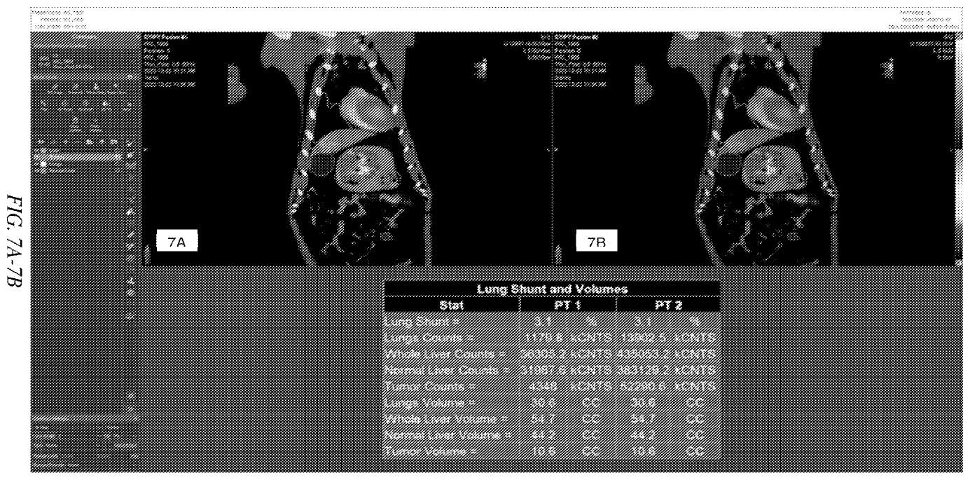

PET/CT imaging was initiated within 5 minutes following microsphere infusion and followed identical imaging protocols for each animal. Results are shown in FIG. 5B. Two separate injections were performed within the PET/CT area. A customized injection apparatus was utilized for performing injections (Boston Scientific, Marlborough, MA), allowing for the controlled delivery of the particles. The first was a “scout dose” injection of up to 1.3 mg of microspheres. Results are shown in FIG. 7A. The second was a dose of up to 13 mg of particles which was designed to model the numbers of particles needed for the therapy procedure (FIGS. 5B and 7B). After each injection, 90 minutes of PET/CT imaging was performed (FIG. 5B).

A dedicated PET docked with multimodality CT was used for preclinical imaging studies. Starting within 1 h post-injection, dynamic and static scans were acquired. Animals were maintained under anesthesia using isoflurane at 1-5% concentration. CT images were acquired for photon attenuation correction and image co-registration with PET imaging data. The reconstruction algorithms for both PET and CT were provided by the scanner manufacturer. The parameters for CT acquisitions were 120 rotation steps over 220°, continuous acquisition, 80 kVp tube voltage, 500 μA tube current, and 175 ms exposure. Image display and analysis were performed using the software package MiM (Cleveland, OH) and Simplicit90Y (Mirada Medical, Denver, CO) (FIGS. 7A and 7B). Volumes of interest (VOIs) were drawn on the co-registered MRI images for tumor, and other organs of interest. VOIs were adjusted to include apparent partial volume spill-outs for organ uptake calculation.

Through this study, it was noted that A) particles functionalized with a therapeutic radioisotope can be successfully delivered to the liver in a catheter directed fashion and B) therapy can be performed using particles functionalized with therapeutic radioisotopes.

What is claimed is:

1. A therapeutic radioisotopic particle, comprising:
at least one therapeutic radioisotope; and
a substrate comprising an inorganic material that comprises metalloid or metal atoms bonded to non-metal atoms, the substrate comprising:
a core extending to a surface, the core comprising a first portion of the metalloid and/or metal atoms bonded to the non-metal atoms and the surface comprising a second portion of the metalloid and/or metal atoms bonded to the non-metal atoms;
wherein the therapeutic radioisotope is bound directly to the substrate through non-metal atoms of the surface of the substrate and/or wherein the therapeutic radioisotope is bound to the substrate through an inorganic bridge comprising non-metal atoms of the surface of the substrate.

2. The particle of claim 1, wherein the substrate comprises a substantially homogeneous mixture of constituent chemical elements.

3. The particle of claim 2, wherein the surface comprises at least a portion of the constituent chemical elements.

4. The particle of any one of claims 1 to 3, wherein the non-metal atoms are oxygen atoms.

5. The particle of claim 4, wherein at least a portion of the oxygen atoms at the surface of the substrate are hydroxyl groups.

6. A therapeutic radioisotopic particle, comprising:
an inorganic substrate with a surface;
at least one therapeutic radioisotope;
wherein the substrate comprises at least one non-metal and at least a metalloid and/or a metal; and
wherein the therapeutic radioisotope is bound to the surface of the substrate by a Lewis acid-base coordination bond to an inorganic Lewis base.

7. A therapeutic radioisotopic particle, comprising:
an inorganic substrate having a surface; and
at least one therapeutic radioisotope;
wherein the substrate comprises at least one non-metal and at least a metalloid and/or a metal; and
wherein the therapeutic radioisotope is bound to the surface of the substrate by a chemical bond to an oxygen of an inorganic species.

8. A therapeutic radioisotopic particle, comprising:
an inorganic substrate comprising a surface having one or more electron donating functionalities; and
at least one therapeutic radioisotope;
wherein the therapeutic radioisotope is bound directly to the surface and/or is bound to the surface through an inorganic bridge during preparation of the therapeutic radioisotopic particle via chemical coupling with the one or more electron donating functionalities.

9. The particle of claim 8, wherein the therapeutic radioisotope is bound directly to the surface of the substrate.

10. The particle of any one of claims 1 to 9, wherein the substrate comprises a metal oxide, a transition metal oxide, a metalloid oxide, or combinations thereof.

11. The particle of any one of claims 1 to 10, wherein the therapeutic radioisotope is bound to the substrate via a chemical bond selected from an ionic bond, a covalent bond, or a coordinate bond.

12. The particle of claim 11, wherein the therapeutic radioisotope is bound via a coordinate bond.

13. A therapeutic radioisotopic particle, comprising:
a ceramic particle substrate, at least one therapeutic radioisotope;
wherein the therapeutic radioisotope is coupled to the surface of the ceramic particle substrate as a Lewis acid-base adduct of an inorganic Lewis base.

14. The particle of any one of claims 6 to 13, wherein the inorganic Lewis base is a component of the substrate and the therapeutic isotope is directly coupled to the substrate surface through the inorganic Lewis base.

15. The particle of claim 13 or 14, wherein the therapeutic radioisotope is coupled to the surface of the ceramic microsphere substrate through an inorganic linker comprising the Lewis base.

16. The particle of claim 15, wherein the inorganic linker is a metal oxide.

17. The particle of claim 16, wherein the metal oxide is a tin oxide.

18. The particle of any of claims 13 to 17, wherein the Lewis base is an oxygen of a metal oxide or metalloid oxide.

19. The particle of any one of claims 6 to 18, wherein the Lewis base is the oxygen of a tin oxide.

20. The particle of any one of claims 1 to 19, wherein the at least one therapeutic radioisotope is a positron emitter or a gamma emitter.

21. The particle of any one of claims 1 to 20, wherein the at least one therapeutic radioisotope is a metallic radioisotope.

22. The particle of any one of claims 1 to 20, wherein the at least one therapeutic radioisotope is selected from ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, ¹⁷⁷Lu, Al¹⁸F, and/or combinations thereof.

23. The particle of any one of claims 1 to 22, wherein the at least one therapeutic radioisotope comprises one or more of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

24. The particle of any one of claims 1 to 22, wherein the at least one therapeutic radioisotope is ¹⁷⁷Lu.

25. The particle of any one of claims 1 to 22, wherein the at least one therapeutic radioisotope is selected from ^{99m}Tc and ⁸⁹Zr.

26. The particle of any one of claims 1 to 25, comprising a structure of Formula (V): 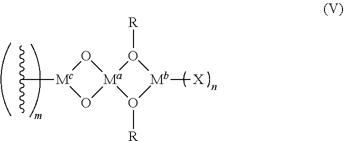
where
the substrate comprises M^{c} and M^{c} is selected from Pb, Al, Si, Y, Mn, Ga, Fe, and Ti;
m is an integer selected from 1, 2, or 3;
M^{b} is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, ¹⁷⁷Lu, Al¹⁸F, and/or combinations thereof;
M^{a} is either an atom of the substrate or a bridging metal atom and M^{a} is selected from Sn, Pb, Al, Si, Y, Mn, Ga, Fe, and Ti;
each instance of R is not present or is —H;
X is selected from —OH, ═O, and —O—; and
n is an integer selected from 0, 1, 2, 3, or 4.

27. The particle of claim 26, wherein:
M^{c} is Al;
the substrate comprises M^{a} and M^{a} is Si;
M^{b} is ⁸⁹Zr;
each X is independently —OH or —O—;
and n is 1 or 2.

28. The particle of claim 26 or 27, wherein M^{b} is ⁸⁹Zr, X is —OH, and n is 2.

29. The particle of claim 26, wherein:
M^{c} is Si;
M^{a} is Sn;
M^{b} is ⁹⁹mTc;
each X is independently —OH or —O—;
and n is 2 or 3.

30. The particle of claim 26, wherein M^{b} is ^{99m}Tc, X is —OH, and n is 3.

31. The particle of any one of claims 1 to 25, comprising a structure of Formula (VIII): 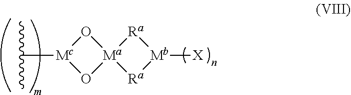
where the substrate comprises M^{a} and M^{c} and where M^{a} and M^{c} are independently selected from Pb, Al, Si, Y, Mn, Ga, Fe, and Ti;
m is an integer selected from 1, 2, or 3;
M^{b} is selected from ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁷⁷Lu, Al¹⁸F, and/or combinations thereof;
each instance of R^{a} is independently OH, O, or —O—Sn(X)ₙ—O—;
X is selected from —OH, ═O, and —O—; and
n is an integer selected from 0, 1, 2, 3, or 4.

32. The particle of claim 31, wherein M^{c} is Al; M^{a} is Si; M^{b} is ^{99m}Tc; each X is independently —OH or ═O; and n is 2 or 3.

33. The particle of claim 31, wherein M^{b} is ^{99m}Tc; at least an instance of R^{a} is —O—Sn(X)ₙ—O—, each X is independently —OH or ═O; and n is 2 or 3.

34. The particle of claim 31, wherein M^{b} is ^{99m}Tc; an instance of R^{a} is —O—Sn—O—; an instance of R^{a} is —O— or —OH—; each X is independently —OH or ═O; and n is 2 or 3.

35. The therapeutic radioisotopic microsphere of any one of claims 1 to 34, wherein the therapeutic radioisotopic microsphere comprises Formula (XI): 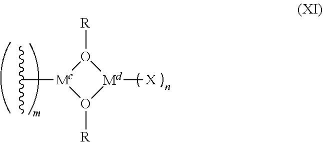
where
the substrate comprises M^{c}, wherein M^{c} is selected from Pb, Al, Si, Y, Mn, Ga, Fe, Ti, Lu, Y, I, Sr, Sm, Ra, At, Ac, Th, and Bi;
m is an integer selected from 1, 2, or 3;
M^{d} is the at least one therapeutic radioisotope, wherein M^{d} is selected from ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, ²¹²Pb, and/or combinations thereof;
each instance of R is —H;
X is —OH; and
n is an integer selected from 0, 1, 2, 3, or 4.

36. The therapeutic radioisotopic microsphere of claim 35, wherein:
M^{c} is selected from Al, Si, and Y; and
M^{d} is ¹⁷⁷Lu.

37. The particle of any one of claims 1 to 45, wherein the substrate comprises at least one non-metal and one or more of a metalloid, a transition metal, and/or a metal.

38. The particle of any one of claims 1 to 37, wherein the substrate comprises a ceramic material.

39. The particle of claim 38, wherein the ceramic comprises at least one element selected from silicon, yttrium, manganese, aluminium, gallium, and titanium.

40. The particle of any one of claims 1 to 39, wherein the substrate comprises glass.

41. The particle of any one of claims 1 to 40, wherein the substrate comprises silicon dioxide and at least one other element selected from manganese, aluminium, gallium, yttrium, boron and titanium.

42. The particle of any one of claims 1 to 41, wherein the substrate comprises SiO₂, Y₂O₃, MnO₂, AlO₃, Ga₂O₃, Fe₂O₃, TiO₂, SrO₂, or combinations thereof.

43. The particle of any one of claims 1 to 42, wherein the substrate comprises SiO₂ and at least one of Y₂O₃, MnO₂, AlO₃, Ga₂O₃, Fe₂O₃, SrO₂, and TiO₂.

44. The particle of any of claims 1 to 43, wherein the substrate comprises an yttrium aluminum silicon oxide.

45. The particle of any one of claims 1 to 44, wherein the particle has a diameter of between 5 μm and 1000 μm.

46. The particle of any one of claims 1 to 44, wherein the particle has a diameter of between 10 nm and 1000 nm.

47. The particle of any one of claims 1 to 46, wherein the substrate is non-porous.

48. The particle of any one of claims 1 to 46, wherein the substrate is porous.

49. A therapeutic radioisotopic particle, comprising:
at least one therapeutic radioisotope; and
a substrate comprising an inorganic material that comprises metalloid or metal atoms bonded to non-metal atoms, the substrate comprising:
a core extending to a surface, the core comprising a first portion of the metalloid and/or metal atoms bonded to the non-metal atoms and the surface comprising a second portion of the metalloid and/or metal atoms bonded to the non-metal atoms;
wherein the therapeutic radioisotope is bound directly to the substrate through non-metal atoms of the surface of the substrate and/or wherein the therapeutic radioisotope is bound to the substrate through an inorganic bridge comprising non-metal atoms of the surface of the substrate.

50. The particle of claim 49, wherein the therapeutic radioisotope is bound directly to the substrate through non-metal atoms of the surface of the substrate.

51. The particle of claim 49 or 50, wherein the substrate is bound to the substrate through an inorganic bridge through non-metal atoms of the surface of the substrate.

52. The particle of any one of claims 49 to 51, wherein the substrate comprises a substantially homogeneous mixture of constituent chemical elements.

53. The particle of claim 52, wherein the surface comprises at least a portion of the constituent chemical elements.

54. The particle of any one of claims 49 to 53, wherein the non-metal atoms are oxygen atoms.

55. The particle of claim 54, wherein at least a portion of the oxygen atoms at the surface of the substrate are hydroxyl groups.

56. A therapeutic radioisotopic particle, comprising:
an inorganic substrate with a surface; and
at least one therapeutic radioisotope;
wherein the substrate comprises at least one non-metal and at least a metalloid and/or a metal; and
wherein the therapeutic radioisotope is bound to the surface of the substrate by a Lewis acid-base coordination bond to an inorganic Lewis base.

57. A therapeutic radioisotopic particle, comprising:
an inorganic substrate having a surface; and
at least one therapeutic radioisotope;
wherein the substrate comprises at least one non-metal and at least a metalloid and/or a metal; and
wherein the therapeutic radioisotope is bound to the surface of the substrate by a chemical bond to an oxygen of an inorganic species.

58. A therapeutic radioisotopic particle, comprising:
an inorganic substrate comprising a surface having one or more electron donating functionalities; and
at least one therapeutic radioisotope;
wherein the therapeutic radioisotope is bound directly to the surface and/or is bound to the surface through an inorganic bridge during preparation of the therapeutic radioisotopic particle via chemical coupling with the one or more electron donating functionalities.

59. The particle of claim 58, wherein the therapeutic radioisotope is bound directly to the surface of the substrate.

60. The particle of any one of claims 49 to 59, wherein the substrate comprises a metal oxide, a transition metal oxide, a metalloid oxide, or combinations thereof.

61. The particle of any one of claims 49 to 60, wherein the therapeutic radioisotope is bound to the substrate via a chemical bond selected from an ionic bond, a covalent bond, or a coordinate bond.

62. The particle of claim 61, wherein the therapeutic radioisotope is bound via a coordinate bond.

63. A therapeutic radioisotopic particle, comprising:
a ceramic particle substrate and at least one therapeutic radioisotope;
wherein the therapeutic radioisotope is coupled to the surface of the ceramic particle substrate as a Lewis acid-base adduct of an inorganic Lewis base.

64. The particle of any one of claims 6 to 63, wherein the inorganic Lewis base is a component of the substrate and the therapeutic radioisotope is directly coupled to the substrate surface through the inorganic Lewis base.

65. The particle of claim 63 or 64, wherein the therapeutic radioisotope is coupled to the surface of the ceramic microsphere substrate through an inorganic linker comprising the Lewis base.

66. The particle of claim 65, wherein the inorganic linker is a metal oxide.

67. The particle of claim 66, wherein the metal oxide is a tin oxide.

68. The particle of any of claims 63 to 67, wherein the Lewis base is an oxygen of a metal oxide or metalloid oxide.

69. The particle of any one of claims 56 to 68, wherein the Lewis base is the oxygen of a tin oxide.

70. The particle of any one of claims 49 to 69, wherein the at least one therapeutic radioisotope is an alpha emitter, a beta emitter, or a positron emitter.

71. The particle of any one of claims 49 to 70, wherein the at least one therapeutic radioisotope is a metallic radioisotope.

72. The therapeutic radioisotopic microsphere of any one of claims 49 to 71, wherein the therapeutic radioisotope is bound directly to the substrate at the surface.

73. The therapeutic radioisotopic microsphere of any one of claims 49 to 72, wherein the at least one therapeutic radioisotope is ¹⁷⁷Lu.

74. The therapeutic radioisotopic microsphere of any one of claims 49 to 73, wherein the at least one therapeutic radioisotope comprises one or more of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

75. The therapeutic radioisotopic microsphere of claim 74, wherein the at least one therapeutic isotope radioisotope is a therapeutic radioisotope.

76. The particle of any one of claims 1 to 75 made by a method comprising:
providing the substrate;
chemically coupling at least one therapeutic radioisotope to the substrate to provide the particle.

77. A radioisotopic particle made by a method comprising:
providing a substrate comprising:
an inorganic material comprising metal or metalloid atoms bonded to non-metal atoms;
a core comprising a first portion of the non-metal atoms; and
a surface comprising a second portion of the non-metal atoms;
providing at least one therapeutic radioisotope; and
chemically coupling the at least one therapeutic radioisotope to the surface layer of the substrate through the second portion of non-metal atoms to provide the therapeutic radioisotopic particle.

78. The radioisotopic particle of claims 76 or 77, wherein the substrate comprises an embedded therapeutic radioisotope.

79. The radioisotopic particle of claims 76 or 77, wherein the substrate comprises a precursor of an embedded therapeutic radioisotope

80. The radioisotopic particle of claim 79, wherein the embedded therapeutic radioisotope is the therapeutic radioisotope.

81. The particle of claim 80, wherein the precursor of the embedded therapeutic radioisotope is activated by neutron bombardment to provide an embedded therapeutic radioisotope.

82. The particle of claims 76 to 81, further comprising providing the at least one therapeutic radioisotope as a salt prior to chemically coupling radioisotope to the surface layer of the inorganic substrate.

83. The particle of claim 77, wherein the salt is an alkali metal salt, an alkali earth metal salt, a halogen salt, a polyatomic salt, or a salt with an organic acid.

84. The particle of claim of 76 to 83, wherein the chemical functionalization is carried out in the presence of a reducing agent.

85. The particle of claim 84, wherein the reducing agent is selected from one or more of a stannous salt, a stannous hydrate, concentrated HCl, sodium borohydride, sodium diothionite, ferrous sulfate, ferric chloride plus ascorbic acid, hypophosphorus acid, and/or hydrazine.

86. The particle of claim of 76 to 84, wherein the radioisotope is ⁹⁹mTc and the chemical functionalization is carried out in the presence of a tin salt.

87. The particle of claim of 86, wherein the radioisotope is provided in the form of ⁹⁹mTc pertechnetate and the chemical functionalization is carried out in the presence of stannous ions.

88. The particle of claim of 76 to 83, wherein the radioisotope is ⁸⁹Zr.

89. The particle of claim of 88, wherein the ⁸⁹Zr is provided in the form of ⁸⁹Zr oxalate.

90. The particle of any one of claims 76 to 89, wherein the substrate comprises a precursor of the therapeutic radioisotope.

91. The particle of claim 90, further comprising exposing the precursor to neutron bombardment to prepare a radioactive radioisotope.

92. A method for preparing a particle, comprising:
providing a ceramic particle substrate; and
reacting the ceramic particle substrate with a therapeutic radioisotope and/or a therapeutic radioisotope under conditions suitable to couple the precursor of the therapeutic radioisotope, and/or the precursor of the therapeutic radioisotope to the surface of the ceramic particle.

93. The method according to claim 92, wherein the substrate comprises an embedded precursor of a therapeutic radioisotope or an embedded therapeutic radioisotope.

94. The method according to claims 92 or 93, further comprising reacting the ceramic particle substrate with a therapeutic radioisotope or a precursor of a therapeutic radioisotope under conditions suitable to couple the precursor of the therapeutic radioisotope or the therapeutic radioisotope to the surface of the ceramic particle.

95. The method according to any of claims 92 to 94, wherein the radioisotope is coupled to the surface of the ceramic particle in the form of a Lewis acid-base adduct.

96. The method according to any of claims 92 to 95, wherein the therapeutic radioisotope is a metallic radio isotope.

97. The method according to any of claims 92 to 96, wherein the therapeutic radioisotope is selected from ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, Al¹⁸F and/or combinations thereof.

98. The method according to any of claims 92 to 97, wherein the therapeutic radioisotope is provided in the form of a salt.

99. The method according to any of claims 92 to 98, wherein the therapeutic radioisotope, and/or precursor thereof is reacted with the ceramic particle in the presence of a reducing agent.

100. The method according to claim 99, wherein the reducing agent is selected from one or more of a stannous salt, a stannous hydrate, HCl, sodium borohydride, sodium diothionite, ferrous sulfate, ferric chloride plus ascorbic acid, hypophosphorus acid, and/or hydrazine.

101. The method according to any of claims 92 to 100, wherein the radioisotope is ⁹⁹mTc.

102. The method according to claim 101, wherein the ^{99m}Tc is provided in the form of a pertechnetate salt.

103. The method according to claim 101, wherein the ⁹⁹mTc is provided in the form of a pertechnetate salt and the reaction is carried out in the presence of stannous ions.

104. The method according to any of claims 92 to 100, wherein the radioisotope is ⁸⁹Zr.

105. The method according to claim 104, wherein the ⁸⁹Zr is provided in the form of ⁸⁹Zr oxalate.

106. The method according to any of claims 92 to 105, wherein the reaction is carried out in the presence of a base.

107. The method according to any of claims 92 to 106, further comprising exposing the particle to neutron bombardment to convert a precursor of therapeutic radioisotope to the therapeutic radioisotope.

108. A method for treating a patient with therapeutic radioisotopic particles, the method comprising:
introducing the population of therapeutic radioisotopic particles to the patient.

109. A method for treating a patient comprising:
introducing the population of therapeutic radioisotopic particles to the patient.

110. The method of claim 109, wherein a target site for treatment in the patient is the liver.

111. The method of claim 110, wherein a target site for treatment is a tumor of the liver of the patient.

112. A method for treating liver cancer in a patient, the method comprising:
providing a population of therapeutic radioisotopic particles;
delivering the population of therapeutic radioisotopic particles to the patient by introducing the population of therapeutic radioisotopic particles to a first position in a vasculature of a body of the patient.

113. The method of claim 112, further comprising administering to the patient the additional amount of therapeutic radioisotopic particles.

114. A kit comprising:
a particle comprising:
a substrate comprising an inorganic material that comprises metalloid or metal atoms bonded to non-metal atoms, the substrate comprising:
a core extending to a surface, the core comprising a first portion of the metalloid or metal atoms bonded to the non-metal atoms and the surface comprising a second portion of the metalloid or metal atoms bonded to the non-metal atoms; and
instructions for reacting a therapeutic radioisotope with the substrate such as to bind the therapeutic radioisotope directly to the substrate through at least a portion of the non-metal atoms at the surface of the substrate.

115. A kit comprising
a particle comprising:
a substrate; wherein the substrate comprises at least one non-metal, a metalloid, or a transition metal oxide; and
instructions for binding a therapeutic radioisotope to the surface of the substrate through a Lewis acid-base coordination bond.

116. A kit comprising a particle comprising a ceramic particle substrate and instructions for carrying out a reaction in which a therapeutic radioisotope is coupled to the ceramic particle substrate as a Lewis acid base adduct.

117. The kit of any one of claims 114 to 116, comprising 50 μl to 2 ml of particles by packed volume, in a sealed unit.

118. The kit of claim 117, wherein the particles are provided in a vial or a syringe.

119. The kit of any one of claims 114 to 118, wherein the therapeutic radioisotope is selected from ⁹⁹mTc, ²⁰¹Th ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr, ¹⁸F, Al¹⁸F, and/or combinations thereof.

120. The kit of any one of claims 114 to 119, further comprising instructions for carrying out a reaction in which a therapeutic radioisotope is coupled to the substrate as a Lewis acid base adduct.

121. The kit of any one of claims 114 to 120, further comprising instructions for activating a precursor to a therapeutic isotope within the substrate to provide a therapeutic isotope.

122. The kit of claim 121, wherein the instructions for activating the precursor to the therapeutic isotope within the substrate indicate that the precursor to the therapeutic isotope is activated prior to coupling the therapeutic radio isotope to the substrate.

123. The kit of any one of claims 114 to 122, wherein the therapeutic radioisotope is one or more of ¹⁷⁷Lu, ⁹⁰Y, ¹³¹I, ⁸⁹Sr, ¹⁵³Sm, ¹⁴⁹Tb, ²²³Ra, ²²⁴Ra, ²¹¹At, ²²⁵Ac, ²²⁷Th, ²¹²Bi, ²¹³Bi, and/or ²¹²Pb.

124. The kit of any one of claims 114 to 123, wherein the kit additionally comprises a reducing agent.

125. The kit of claim 124, wherein the reducing agent is selected from one or more of a stannous salt, concentrated HCl, sodium borohydride, sodium diothionite, ferrous sulfate, ferric chloride plus ascorbic acid, hypophosphorus acid, and/or hydrazine.

126. The kit of claims 124 to 125, wherein the reducing agent is a stannous salt, the radioisotope is ⁹⁹mTc and the radioisotope is in the form of a pertechnetate salt.

127. The kit of any one of claims 114 to 126, wherein the therapeutic radioisotope is ⁸⁹Zr.

128. The kit of claim 127, wherein the therapeutic radioisotope is in the form of ⁸⁹Zr zirconium oxalate

129. The kit of any one of claims 114 to 128, wherein the therapeutic radioisotope is ¹⁷⁷Lu.

130. The kit of any one of claims 114 to 129, wherein the substrate comprises a yttrium oxide aluminosilicate.

131. The kit of any one of claims 114 to 130, further comprising one or more of a vascular access needle, a vascular guidewire, a vascular sheath (e.g., 4-6Fr), a vascular catheter (4-5Fr), a microcatheter, syringes, and a vial.
